(19) United States
(12) Patent Application Publication
(10) Pub. No.: US 2004/0157848 A1
(43) Pub. Date: Aug. 12, 2004

(54) Methods and compositions for the treatment of herpes virus infections using cyclooxygenase-2 selective inhibitors or cyclooxygenase-2 inhibitors in combination with antiviral agents

(75) Inventor: Timothy Maziasz, Northbrook, IL (US)

Correspondence Address: SENNIGER POWERS LEAVITT AND ROEDEL, ONE METROPOLITAN SQUARE, 16TH FLOOR, ST LOUIS, MO, 63102, US

(73) Assignee: Pharmacia Corporation

(21) Appl. No.: 10/742,400

(22) Filed: Dec. 19, 2003

Related U.S. Application Data
(60) Provisional application No. 60/435,392, filed on Dec. 19, 2002.

(51) Int. Cl. A61K 31/50; A61K 31/415; A61K 31/18

(52) U.S. Cl. 514/247; 514/374; 514/406; 514/602

(57) ABSTRACT

The present invention provides compositions and methods for the treatment of herpes virus infections. In one aspect, the invention provides a combination therapy for treating a herpes virus infection comprising the administration to a subject of an anti-herpes virus agent in combination with a cyclooxygenase-2 selective inhibitor. In another aspect, the invention provides a mono therapy for treating a herpes virus infection comprising administering a cyclooxygenase-2 selective inhibitor to a subject.

### CROSS-REFERENCE TO RELATED APPLICATION

This application claims priority from Provisional Application Serial No. 60/435,392 filed on Dec. 19, 2002, which is hereby incorporated by reference in its entirety.

### FIELD OF INVENTION

The present invention provides methods and compositions related to the treatment of herpes virus infections as well as related conditions. In one aspect, the invention is directed toward a method of treating a herpes virus infection comprising the administration to a subject of a cyclooxygenase-2 selective inhibitor. In another aspect, the invention is directed toward a combination therapy for the treatment of a herpes virus infection comprising the administration to a subject of a cyclooxygenase-2 selective inhibitor in combination with an anti-herpes virus agent that is not a cyclooxygenase-2 selective inhibitor.

### BACKGROUND OF THE INVENTION

At least four of the human herpes viruses, including herpes simplex virus type 1 (HSV-1), herpes simplex virus type 2 (HSV-2), cytomegalovirus (CMV), and varicella zoster virus (VZV) are known to infect and cause lesions in humans. Together, these four viruses are the leading cause of infectious blindness in the developed world. HSV-1 primarily infects the oral cavity, while HSV-2 primarily infects genital sites. But any area of the body, including the eye, skin and brain, can be infected with either type of HSV. Generally speaking, HSV is transmitted to a non-infected individual by direct contact with the infected site of the infected individual. The initial symptoms of a primary or recurrent HSV infection include tingling, pain, and/or paraesthesia at the site of infection. This is followed by formation of a lesion at the infected site, such as in the oral cavity, eye, skin, or reproductive tract. Healing of lesions typically occurs in approximately ten to fourteen days.

While the immune reaction that occurs in response to a HSV infection typically prevents dissemination of the virus throughout the body of an immmunocompetent individual, it does not eliminate all infectious HSV particles. The virus particles that are not killed by the immune response move along the nerve path to the ganglia of the infected individual where they may remain in a state of latency. In response to a variety of stimuli including stress, environmental factors, other medications, food additives or food substances, the infectious virus particles may leave the ganglia and cause a periodic recurrent infection at or near the original site of infection for the lifetime of the individual. In those HSV-infected individuals who are immunosuppressed or who lack a well-developed immune system, such as neonates, dissemination of the virus particles from the infected site can also occur and lead to life-threatening complications, including encephalitis.

The continued spread of the HSV epidemic in both the industrialized countries and the developing world provides compelling evidence that there is a continuing need for better anti-HSV treatments and for better anti-HSV drugs. Although HSV transmission is in theory largely preventable, in practice, without the development of better anti-HSV treatments and better anti-HSV drugs, HSV will continue to infect millions throughout the world. While programs to reduce transmission of HSV have achieved some success, it is unlikely that widespread application of these programs will be able to achieve a sustained decrease in HSV transmission.

Despite the urgent need for new anti-HSV drugs, which are both safe and effective, progress toward achieving this goal has been frustratingly slow. Since there is no known cure for the disease itself, traditional medical therapy is directed to reducing the degree of pain or discomfort associated with the eruption. Among these, a nucleoside analog, such as acyclovir, famciclovir, or valaciclovir, has been shown to be fairly effective. Also, idoxuridiene and trifluridine are used on superficial infections. Treatment for HSV-1, which normally manifest as outbreaks referred to as “cold sores” or “fever blisters,” are traditionally treated with various moistening cremes, balms or ointments that are sold over-the-counter. Some desiccants, such as camphor, may also be used in treating HSV-1. Although these compounds have great value in lessening the pain attendant to the herpes event, there remains a need for improved medications that may be effective in treating the symptoms arising from outbreaks of HSV infections, as well as methods of eliminating the virus from the body of the infected individual altogether.

Recent studies indicate that herpes virus infection may involve an inflammatory component. It has been disclosed that cyclooxygenase-2 mRNA, protein, and activity are transiently induced after infection of human fibroblasts with a cytomegalovirus (Zhu et al., (2002) PNAS 99(6):3932-45). Further prostaglandin E₂ levels (a product of cyclooxygenase-2 activity) increased by a factor of 50 in cultures of the fibroblasts infected by the virus (Zhu et al., (2002) supra). Treatment of the cytomegalovirus infected fibroblasts with a cyclooxygenase-2 selective inhibitor, however, reduced the yield of virus in the cells by a factor of about 100 (Zhu et al., (2002) supra). Moreover, is has also been disclosed that treatment of cytomegalovirus infected human smooth muscle with aspirin reduces cytomegalovirus induced reactive oxygen species (Speir et al., (1998) Circulation Res. 83:210-216).

Generally speaking, traditional NSAIDs, such as aspirin, are active in reducing the prostaglandin-induced pain and swelling associated with the inflammation process. But the use of high doses of most common NSAIDs can produce severe side effects, including life-threatening ulcers that limit their therapeutic potential. One reason proposed for the severe side effects associated with traditional NSAIDs is their non-selective inhibition of both of the cyclooxygenase enzymes (COX), commonly known as COX-1 and COX-2. COX-1 is constitutively expressed and mediates a number of physiological functions, such as kidney and gastrointestinal function.

COX-2, contrastingly, is induced in response to an inflammation-mediated event. While conventional NSAIDs block both forms of the enzyme, a newer class of NSAID, selective cyclooxygenase-2 inhibitors, provides a viable target of inhibition that more effectively reduces inflammation and produces fewer and less drastic side effects. Compounds that selectively inhibit cyclooxygenase-2 have been described in U.S. Pat. Nos. 5,380,738; 5,344,991; 5,393,790; 5,434,178; 5,474,995; 5,510,368 and WO documents WO96/06840, WO96/03388, WO96/03387, WO96/19469, WO96/25405, WO95/15316, WO94/15932, WO94/27980, WO95/00501, WO94/13635, WO94/20480, and WO94/26731.

### SUMMARY OF THE INVENTION

Among the several aspects of the invention is provided a method and a composition for the treatment of a herpes virus infection as well as associated diseases and related disorders. In one aspect of the invention, the method comprises administering to the subject a cyclooxygenase-2 selective inhibitor or an isomer, a pharmaceutically acceptable salt, ester, or prodrug thereof and an anti-herpes virus agent or an isomer, a pharmaceutically acceptable salt, ester, or prodrug thereof, wherein the anti-herpes agent is not a cyclooxygenase-2 selective inhibitor. In another aspect of the invention, the method comprises administering a cyclooxygenase-2 selective inhibitor or an isomer, a pharmaceutically acceptable salt, ester, or prodrug thereof to a subject.

In one embodiment, the cyclooxygenase-2 selective inhibitor is a member of the chromene class of compounds. For example, the chromene compound may be a compound of the formula:

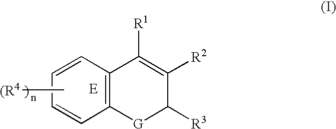

wherein:

n is an integer which is 0, 1, 2, 3 or 4;

G is O, S or NR^{a};

R^{a} is alkyl;

R¹ is selected from the group consisting of H and aryl;

R² is selected from the group consisting of carboxyl, aminocarbonyl, alkylsulfonylaminocarbonyl and alkoxycarbonyl;

R³ is selected from the group consisting of haloalkyl, alkyl, aralkyl, cycloalkyl and aryl optionally substituted with one or more radicals selected from alkylthio, nitro and alkylsulfonyl; and

each R⁴ is independently selected from the group consisting of H, halo, alkyl, aralkyl, alkoxy, aryloxy, heteroaryloxy, aralkyloxy, heteroaralkyloxy, haloalkyl, haloalkoxy, alkylamino, arylamino, aralkylamino, heteroarylamino, heteroarylalkylamino, nitro, amino, aminosulfonyl, alkylaminosulfonyl, arylaminosulfonyl, heteroarylaminosulfonyl, aralkylaminosulfonyl, heteroaralkylaminosulfonyl, heterocyclosulfonyl, alkylsulfonyl, hydroxyarylcarbonyl, nitroaryl, optionally substituted aryl, optionally substituted heteroaryl, aralkylcarbonyl, heteroarylcarbonyl, arylcarbonyl, aminocarbonyl, and alkylcarbonyl;

or R⁴ together with the carbon atoms to which it is attached and the remainder of ring E forms a naphthyl radical.

In another embodiment, the cyclooxygenase-2 selective inhibitor comprises a compound having the formula

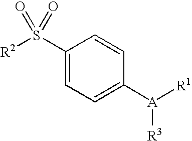

wherein:

A is selected from the group consisting of partially unsaturated or unsaturated heterocyclyl and partially unsaturated or unsaturated carbocyclic rings;

R¹ is selected from the group consisting of heterocyclyl, cycloalkyl, cycloalkenyl and aryl, wherein R¹ is optionally substituted at a substitutable position with one or more radicals selected from alkyl, haloalkyl, cyano, carboxyl, alkoxycarbonyl, hydroxyl, hydroxyalkyl, haloalkoxy, amino, alkylamino, arylamino, nitro, alkoxyalkyl, alkylsulfinyl, halo, alkoxy and alkylthio;

R² is selected from the group consisting of methyl or amino; and

R³ is selected from the group consisting of a radical selected from H, halo, alkyl, alkenyl, alkynyl, oxo, cyano, carboxyl, cyanoalkyl, heterocyclyloxy, alkyloxy, alkylthio, alkylcarbonyl, cycloalkyl, aryl, haloalkyl, heterocyclyl, cycloalkenyl, aralkyl, heterocyclylalkyl, acyl, alkylthioalkyl, hydroxyalkyl, alkoxycarbonyl, arylcarbonyl, aralkylcarbonyl, aralkenyl, alkoxyalkyl, arylthioalkyl, aryloxyalkyl, aralkylthioalkyl, aralkoxyalkyl, alkoxyaralkoxyalkyl, alkoxycarbonylalkyl, aminocarbonyl, aminocarbonylalkyl, alkylaminocarbonyl, N-arylaminocarbonyl, N-alkyl-N-arylaminocarbonyl, alkylaminocarbonylalkyl, carboxyalkyl, alkylamino, N-arylamino, N-aralkylamino, N-alkyl-N-aralkylamino, N-alkyl-N-arylamino, aminoalkyl, alkylaminoalkyl, N-arylaminoalkyl, N-aralkylaminoalkyl, N-alkyl-N-aralkylaminoalkyl, N-alkyl-N-arylaminoalkyl, aryloxy, aralkoxy, arylthio, aralkylthio, alkylsulfinyl, alkylsulfonyl, aminosulfonyl, alkylaminosulfonyl, N-arylaminosulfonyl, arylsulfonyl, N-alkyl-N-arylaminosulfonyl.

In still a further embodiment, the anti-herpes virus agent is selected from the group consisting of viral cellular entry inhibitors, viral replication inhibitors, viral assembly inhibitors, human immune enhancing agents, virucidal agents, and antimitotic agents.

Other aspects and embodiments of the invention are more thoroughly detailed below.

### ABBREVIATIONS AND DEFINITIONS

The term “acyl” is a radical provided by the residue after removal of hydroxyl from an organic acid. Examples of such acyl radicals include alkanoyl and aroyl radicals. Examples of such lower alkanoyl radicals include formyl, acetyl, propionyl, butyryl, isobutyryl, valeryl, isovaleryl, pivaloyl, hexanoyl, and trifluoroacetyl.

The term “alkenyl” is a linear or branched radical having at least one carbon-carbon double bond of two to about twenty carbon atoms or, preferably, two to about twelve carbon atoms. More preferred alkyl radicals are “lower alkenyl” radicals having two to about six carbon atoms. Examples of alkenyl radicals include ethenyl, propenyl, allyl, propenyl, butenyl and 4-methylbutenyl.

The terms “alkenyl” and “lower alkenyl” also are radicals having “cis” and “trans” orientations, or alternatively, “E” and “Z” orientations. The term “cycloalkyl” is a saturated carbocyclic radical having three to twelve carbon atoms. More preferred cycloalkyl radicals are “lower cycloalkyl” radicals having three to about eight carbon atoms. Examples of such radicals include cyclopropyl, cyclobutyl, cyclopentyl and cyclohexyl.

The terms “alkoxy” and “alkyloxy” are linear or branched oxy-containing radicals each having alkyl portions of one to about ten carbon atoms. More preferred alkoxy radicals are “lower alkoxy” radicals having one to six carbon atoms. Examples of such radicals include methoxy, ethoxy, propoxy, butoxy and tert-butoxy.

The term “alkoxyalkyl” is an alkyl radical having one or more alkoxy radicals attached to the alkyl radical, that is, to form monoalkoxyalkyl and dialkoxyalkyl radicals. The “alkoxy” radicals may be further substituted with one or more halo atoms, such as fluoro, chloro or bromo, to provide haloalkoxy radicals. More preferred haloalkoxy radicals are “lower haloalkoxy” radicals having one to six carbon atoms and one or more halo radicals. Examples of such radicals include fluoromethoxy, chloromethoxy, trifluoromethoxy, trifluoroethoxy, fluoroethoxy and fluoropropoxy.

The term “alkoxycarbonyl” is a radical containing an alkoxy radical, as defined above, attached via an oxygen atom to a carbonyl radical. More preferred are “lower alkoxycarbonyl” radicals with alkyl porions having 1 to 6 carbons. Examples of such lower alkoxycarbonyl (ester) radicals include substituted or unsubstituted methoxycarbonyl, ethoxycarbonyl, propoxycarbonyl, butoxycarbonyl and hexyloxycarbonyl.

Where used, either alone or within other terms such as “haloalkyl”, “alkylsulfonyl”, “alkoxyalkyl” and “hydroxyalkyl”, the term “alkyl” is a linear, cyclic or branched radical having one to about twenty carbon atoms or, preferably, one to about twelve carbon atoms. More preferred alkyl radicals are “lower alkyl” radicals having one to about ten carbon atoms. Most preferred are lower alkyl radicals having one to about six carbon atoms. Examples of such radicals include methyl, ethyl, n-propyl, isopropyl, n-butyl, isobutyl, sec-butyl, tert-butyl, pentyl, iso-amyl, hexyl and the like.

The term “alkylamino” is an amino group that has been substituted with one or two alkyl radicals. Preferred are “lower N-alkylamino” radicals having alkyl portions having 1 to 6 carbon atoms. Suitable lower alkylamino may be mono or dialkylamino such as N-methylamino, N-ethylamino, N,N-dimethylamino, N,N-diethylamino or the like.

The term “alkylaminoalkyl” is a radical having one or more alkyl radicals attached to an aminoalkyl radical.

The term “alkylaminocarbonyl” is an aminocarbonyl group that has been substituted with one or two alkyl radicals on the amino nitrogen atom. Preferred are “N-alkylaminocarbonyl” “N,N-dialkylaminocarbonyl” radicals. More preferred are “lower N-alkylaminocarbonyl” “lower N,N-dialkylaminocarbonyl” radicals with lower alkyl portions as defined above.

The terms “alkylcarbonyl”, “arylcarbonyl” and “aralkylcarbonyl” include radicals having alkyl, aryl and aralkyl radicals, as defined above, attached to a carbonyl radical. Examples of such radicals include substituted or unsubstituted methylcarbonyl, ethylcarbonyl, phenylcarbonyl and benzylcarbonyl.

The term “alkylthio” is a radical containing a linear or branched alkyl radical, of one to about ten carbon atoms attached to a divalent sulfur atom. More preferred alkylthio radicals are “lower alkylthio” radicals having alkyl radicals of one to six carbon atoms. Examples of such lower alkylthio radicals are methylthio, ethylthio, propylthio, butylthio and hexylthio.

The term “alkylthioalkyl” is a radical containing an alkylthio radical attached through the divalent sulfur atom to an alkyl radical of one to about ten carbon atoms. More preferred alkylthioalkyl radicals are “lower alkylthioalkyl” radicals having alkyl radicals of one to six carbon atoms. Examples of such lower alkylthioalkyl radicals include methylthiomethyl.

The term “alkylsulfinyl” is a radical containing a linear or branched alkyl radical, of one to ten carbon atoms, attached to a divalent —S(═O)— radical. More preferred alkylsulfinyl radicals are “lower alkylsulfinyl” radicals having alkyl radicals of one to six carbon atoms. Examples of such lower alkylsulfinyl radicals include methylsulfinyl, ethylsulfinyl, butylsulfinyl and hexylsulfinyl.

The term “alkynyl” is a linear or branched radical having two to about twenty carbon atoms or, preferably, two to about twelve carbon atoms. More preferred alkynyl radicals are “lower alkynyl” radicals having two to about ten carbon atoms. Most preferred are lower alkynyl radicals having two to about six carbon atoms. Examples of such radicals include propargyl, butynyl, and the like.

The term “aminoalkyl” is an alkyl radical substituted with one or more amino radicals. More preferred are “lower aminoalkyl” radicals. Examples of such radicals include aminomethyl, aminoethyl, and the like.

The term “aminocarbonyl” is an amide group of the formula —C(═O)NH₂.

The term “aralkoxy” is an aralkyl radical attached through an oxygen atom to other radicals.

The term “aralkoxyalkyl” is an aralkoxy radical attached through an oxygen atom to an alkyl radical.

The term “aralkyl” is an aryl-substituted alkyl radical such as benzyl, diphenylmethyl, triphenylmethyl, phenylethyl, and diphenylethyl. The aryl in said aralkyl may be additionally substituted with halo, alkyl, alkoxy, halkoalkyl and haloalkoxy. The terms benzyl and phenylmethyl are interchangeable.

The term “aralkylamino” is an aralkyl radical attached through an amino nitrogen atom to other radicals. The terms “N-arylaminoalkyl” and “N-aryl-N-alkyl-aminoalkyl” are amino groups which have been substituted with one aryl radical or one aryl and one alkyl radical, respectively, and having the amino group attached to an alkyl radical. Examples of such radicals include N-phenylaminomethyl and N-phenyl-N-methylaminomethyl.

The term “aralkylthio” is an aralkyl radical attached to a sulfur atom.

The term “aralkylthioalkyl” is an aralkylthio radical attached through a sulfur atom to an alkyl radical.

The term “aroyl” is an aryl radical with a carbonyl radical as defined above. Examples of aroyl include benzoyl, naphthoyl, and the like and the aryl in said aroyl may be additionally substituted.

The term “aryl”, alone or in combination, is a carbocyclic aromatic system containing one, two or three rings wherein such rings may be attached together in a pendent manner or may be fused. The term “aryl” includes aromatic radicals such as phenyl, naphthyl, tetrahydronaphthyl, indane and biphenyl. Aryl moieties may also be substituted at a substitutable position with one or more substituents selected independently from alkyl, alkoxyalkyl, alkylaminoalkyl, carboxyalkyl, alkoxycarbonylalkyl, aminocarbonylalkyl, alkoxy, aralkoxy, hydroxyl, amino, halo, nitro, alkylamino, acyl, cyano, carboxy, aminocarbonyl, alkoxycarbonyl and aralkoxycarbonyl.

The term “arylamino” is an amino group, which has been substituted with one or two aryl radicals, such as N-phenylamino. The “arylamino” radicals may be further substituted on the aryl ring portion of the radical.

The term “aryloxyalkyl” is a radical having an aryl radical attached to an alkyl radical through a divalent oxygen atom.

The term “arylthioalkyl” is a radical having an aryl radical attached to an alkyl radical through a divalent sulfur atom.

The term “carbonyl”, whether used alone or with other terms, such as “alkoxycarbonyl”, is —(C═O)—.

The terms “carboxy” or “carboxyl”, whether used alone or with other terms, such as “carboxyalkyl”, is —CO₂H.

The term “carboxyalkyl” is an alkyl radical substituted with a carboxy radical. More preferred are “lower carboxyalkyl” which are lower alkyl radicals as defined above, and may be additionally substituted on the alkyl radical with halo. Examples of such lower carboxyalkyl radicals include carboxymethyl, carboxyethyl and carboxypropyl.

The term “cycloalkenyl” is a partially unsaturated carbocyclic radical having three to twelve carbon atoms. More preferred cycloalkenyl radicals are “lower cycloalkenyl” radicals having four to about eight carbon atoms. Examples of such radicals include cyclobutenyl, cyclopentenyl, cyclopentadienyl, and cyclohexenyl.

The term “cyclooxygenase-2 selective inhibitor” is a compound able to inhibit cyclooxygenase-2 without significant inhibition of cyclooxygenase-1. Typically, it includes compounds that have a cyclooxygenase-2 IC₅₀ of less than about 0.2 micro molar, and also have a selectivity ratio of cyclooxygenase-2 inhibition over cyclooxygenase-1 inhibition of at least 50, and more typically, of at least 100. Even more typically, the compounds have a cyclooxygenase-1 IC₅₀ of greater than about 1 micro molar, and more preferably of greater than 10 micro molar. Inhibitors of the cyclooxygenase pathway in the metabolism of arachidonic acid used in the present method may inhibit enzyme activity through a variety of mechanisms. By the way of example, and without limitation, the inhibitors used in the methods described herein may block the enzyme activity directly by acting as a substrate for the enzyme.

The term “halo” is a halogen such as fluorine, chlorine, bromine or iodine.

The term “haloalkyl” is a radical wherein any one or more of the alkyl carbon atoms is substituted with halo as defined above. Specifically included are monohaloalkyl, dihaloalkyl and polyhaloalkyl radicals. A monohaloalkyl radical, for one example, may have either an iodo, bromo, chloro or fluoro atom within the radical. Dihalo and polyhaloalkyl radicals may have two or more of the same halo atoms or a combination of different halo radicals. “Lower haloalkyl” is a radical having 1-6 carbon atoms. Examples of haloalkyl radicals include fluoromethyl, difluoromethyl, trifluoromethyl, chloromethyl, dichloromethyl, trichloromethyl, trichloromethyl, pentafluoroethyl, heptafluoropropyl, difluorochloromethyl, dichlorofluoromethyl, difluoroethyl, difluoropropyl, dichloroethyl and dichloropropyl.

The term “heteroaryl” is an unsaturated heterocyclyl radical. Examples of unsaturated heterocyclyl radicals, also termed “heteroaryl” radicals include unsaturated 3 to 6 membered heteromonocyclic group containing 1 to 4 nitrogen atoms, for example, pyrrolyl, pyrrolinyl, imidazolyl, pyrazolyl, pyridyl, pyrimidyl, pyrazinyl, pyridazinyl, triazolyl (e.g., 4H-1,2,4-triazolyl, 1H-1,2,3-triazolyl, 2H-1,2,3-triazolyl, etc.) tetrazolyl (e.g. 1H-tetrazolyl, 2H-tetrazolyl, etc.), etc.; unsaturated condensed heterocyclyl group containing 1 to 5 nitrogen atoms, for example, indolyl, isoindolyl, indolizinyl, benzimidazolyl, quinolyl, isoquinolyl, indazolyl, benzotriazolyl, tetrazolopyridazinyl (e.g., tetrazolo[1,5-b]pyridazinyl, etc.), etc.; unsaturated 3 to 6-membered heteromonocyclic group containing an oxygen atom, for example, pyranyl, furyl, etc.; unsaturated 3 to 6-membered heteromonocyclic group containing a sulfur atom, for example, thienyl, etc.; unsaturated 3- to 6-membered heteromonocyclic group containing 1 to 2 oxygen atoms and 1 to 3 nitrogen atoms, for example, oxazolyl, isoxazolyl, oxadiazolyl (e.g., 1,2,4-oxadiazolyl, 1,3,4-oxadiazolyl, 1,2,5-oxadiazolyl, etc.) etc.; unsaturated condensed heterocyclyl group containing 1 to 2 oxygen atoms and 1 to 3 nitrogen atoms (e.g. benzoxazolyl, benzoxadiazolyl, etc.); unsaturated 3 to 6-membered heteromonocyclic group containing 1 to 2 sulfur atoms and 1 to 3 nitrogen atoms, for example, thiazolyl, thiadiazolyl (e.g., 1,2,4-thiadiazolyl, 1,3,4-thiadiazolyl, 1,2,5-thiadiazolyl, etc.) etc.; unsaturated condensed heterocyclyl group containing 1 to 2 sulfur atoms and 1 to 3 nitrogen atoms (e.g., benzothiazolyl, benzothiadiazolyl, etc.) and the like. The term also includes radicals where heterocyclyl radicals are fused with aryl radicals. Examples of such fused bicyclic radicals include benzofuran, benzothiophene, and the like. Said “heterocyclyl group” may have 1 to 3 substituents such as alkyl, hydroxyl, halo, alkoxy, oxo, amino and alkylamino.

The term “heterocyclyl” is a saturated, partially unsaturated and unsaturated heteroatom-containing ring-shaped radical, where the heteroatoms may be selected from nitrogen, sulfur and oxygen. Examples of saturated heterocyclyl radicals include saturated 3 to 6-membered heteromonocylic group containing 1 to 4 nitrogen atoms (e.g. pyrrolidinyl, imidazolidinyl, piperidino, piperazinyl, etc.); saturated 3 to 6-membered heteromonocyclic group containing 1 to 2 oxygen atoms and 1 to 3 nitrogen atoms (e.g. morpholinyl, etc.); saturated 3 to 6-membered heteromonocyclic group containing 1 to 2 sulfur atoms and 1 to 3 nitrogen atoms (e.g., thiazolidinyl, etc.). Examples of partially unsaturated heterocyclyl radicals include dihydrothiophene, dihydropyran, dihydrofuran and dihydrothiazole.

The term “heterocyclylalkyl” is a saturated and partially unsaturated heterocyclyl-substituted alkyl radical, such as pyrrolidinyhnethyl, and heteroaryl-substituted alkyl radicals, such as pyridylmethyl, quinolylmethyl, thienylmethyl, furylethyl, and quinolylethyl. The heteroaryl in said heteroaralkyl may be additionally substituted with halo, alkyl, alkoxy, halkoalkyl and haloalkoxy.

The phrase “herpes virus infection” means the presence of herpes virus in a subject, irrespective of the stage of infection or degree of colonization.

The phrase “herpes virus associated disease or related disorder” encompasses a number of different kinds of diseases or related disorder caused by or resulting from herpes virus infection.

The term “hydrido” is a single hydrogen atom (H). This hydrido radical may be attached, for example, to an oxygen atom to form a hydroxyl radical or two hydrido radicals may be attached to a carbon atom to form a methylene (—CH₂—) radical.

The term “hydroxyalkyl” is a linear or branched alkyl radical having one to about ten carbon atoms any one of which may be substituted with one or more hydroxyl radicals. More preferred hydroxyalkyl radicals are “lower hydroxyalkyl” radicals having one to six carbon atoms and one or more hydroxyl radicals. Examples of such radicals include hydroxymethyl, hydroxyethyl, hydroxypropyl, hydroxybutyl and hydroxyhexyl.

The term “inhibition” as used herein means a decrease in the severity of a herpes virus infection as compared to that which would occur in the absence of the administration of the composition of the invention to a subject. This decrease in severity may result from a reduction in viral number, a reduction in viral replication, a reduction in the subject's cell growth infected with the virus, a reduction in cellular replication in the subject, a reduction in cellular mitosis in a subject, a reduction in viral colonization or any combination thereof.

The term “pharmaceutically acceptable” is used adjectivally herein to mean that the modified noun is appropriate for use in a pharmaceutical product; that is the “pharmaceutically acceptable” material is relatively safe and/or non-toxic, though not necessarily providing a separable therapeutic benefit by itself. Pharmaceutically acceptable cations include metallic ions and organic ions. More preferred metallic ions include, but are not limited to appropriate alkali metal salts, alkaline earth metal salts and other physiologically acceptable metal ions. Exemplary ions include aluminum, calcium, lithium, magnesium, potassium, sodium and zinc in their usual valences. Preferred organic ions include protonated tertiary amines and quaternary ammonium cations, including in part, trimethylamine, diethylamine, N,N′-dibenzylethylenediamine, chloroprocaine, choline, diethanolamine, ethylenediamine, meglumine (N-methylglucamine) and procaine. Exemplary pharmaceutically acceptable acids include without limitation hydrochloric acid, hydrobromic acid, phosphoric acid, sulfuric acid, methanesulfonic acid, acetic acid, formic acid, tartaric acid, maleic acid, malic acid, citric acid, isocitric acid, succinic acid, lactic acid, gluconic acid, glucuronic acid, pyruvic acid, oxalacetic acid, fumaric acid, propionic acid, aspartic acid, glutamic acid, benzoic acid, and the like.

The term “prevention” includes one or more of the following: (1) substantially preventing the onset of a clinically evident herpes virus infection in a subject; (2) preventing the onset of a preclinically evident stage of a herpes virus infection in a subject; or (3) substantially preventing herpes virus colonization in a subject. This definition includes prophylactic treatment.

The term “prodrug” refers to a chemical compound that can be converted into a therapeutic compound by metabolic or simple chemical processes within the body of the subject. For example, a class of prodrugs of COX-2 inhibitors is described in U.S. Pat. No. 5,932,598, herein incorporated by reference.

The term “reduced cell growth” is intended to include a reduction in cell growth including the complete cessation of cell growth causing, e.g., apoptosis, in one or more herpes virus-infected cells.

The term “subject” for purposes of treatment or prevention includes a species that is susceptible to herpes virus infection. In one embodiment, the subject is a human. In another embodiment, the subject is a companion animal such as a dog or cat.

The term “sulfonyl”, whether used alone or linked to other terms such as alkylsulfonyl, is a divalent radical —SO₂—. “Alkylsulfonyl” is an alkyl radical attached to a sulfonyl radical, where alkyl is defined as above. More preferred alkylsulfonyl radicals are “lower alkylsulfonyl” radicals having one to six carbon atoms. Examples of such lower alkylsulfonyl radicals include methylsulfonyl, ethylsulfonyl and propylsulfonyl. The “alkylsulfonyl” radicals may be further substituted with one or more halo atoms, such as fluoro, chloro or bromo, to provide haloalkylsulfonyl radicals. The terms “sulfamyl”, “aminosulfonyl” and “sulfonamidyl” are NH₂O₂S—.

The phrase “therapeutically-effective” is intended to qualify the amount of agent (i.e. cyclooxygenase-2 selective inhibitor or anti-herpes virus agent) which will achieve the goal of improvement in disorder severity and the frequency of incidence over no treatment.

### DESCRIPTION OF THE PREFERRED EMBODIMENTS

The present invention provides a monotherapy and a combination therapy that may be utilized to treat a herpes virus infection in a subject. In one aspect of the invention, the monotherapy comprises administering a therapeutically effective amount of a cyclooxygenase-2 selective inhibitor to a subject. In another aspect, the combination therapy comprises the administration to a subject of a therapeutically effective amount of a COX-2 selective inhibitor in combination with a therapeutically effective amount of an anti-herpes virus agent, wherein the anti-herpes virus is not a cyclooxygenase-2 selective inhibitor. The monotherapy and combination therapy may be employed not only to treat or prevent herpes virus (herpes virus) infection, but also conditions resulting from herpes virus infection.

### Cyclooxygenase-2 Selective Inhibitors for Use in Monotherapy or Combination Therapy

A number of suitable cyclooxygenase-2 selective inhibitors or an isomer, a pharmaceutically acceptable salt, ester, or prodrug thereof may be employed in a composition of the current invention. In one embodiment, the cyclooxygenase-2 selective inhibitor can be, for example, the cyclooxygenase-2 selective inhibitor meloxicam, Formula B-1 (CAS registry number 71125-38-7) or an isomer, a pharmaceutically acceptable salt, ester, or prodrug of a compound having Formula B-1.

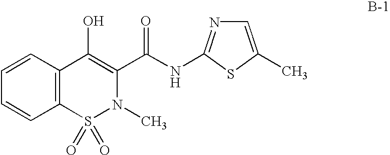

In yet another embodiment, the cyclooxygenase-2 selective inhibitor is the cyclooxygenase-2 selective inhibitor, 6-[[5-(4-chlorobenzoyl)-1,4-dimethyl-1H-pyrrol-2-yl]methyl]-3(2H)-pyridazinone, Formula B-2 (CAS registry number 179382-91-3) or an isomer, a pharmaceutically acceptable salt, ester, or prodrug of a compound having Formula B-2.

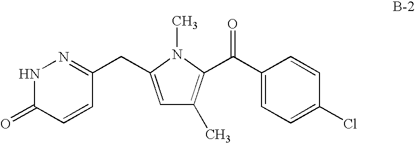

In still another embodiment the cyclooxygenase-2 selective inhibitor is a chromene compound that is a substituted benzopyran or a substituted benzopyran analog, and even more typically, selected from the group consisting of substituted benzothiopyrans, dihydroquinolines, dihydronaphthalenes or a compound having Formula I shown below and possessing, by way of example and not limitation, the structures disclosed in Table 1x. Furthermore, benzopyran cyclooxygenase-2 selective inhibitors useful in the practice of the present methods are described in U.S. Pat. Nos. 6,034,256 and 6,077,850 herein incorporated by reference in their entirety.

In another embodiment, the cyclooxygenase-2 selective inhibitor is a chromene compound represented by Formula I or an isomer, a pharmaceutically acceptable salt, ester, or prodrug thereof:

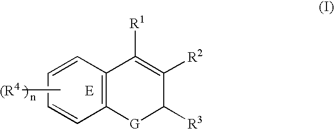

wherein

n is an integer which is 0, 1, 2, 3 or 4;

G is O, S or NR^{a};

R^{a} is alkyl;

R¹ is selected from the group consisting of H and aryl;

R² is selected from the group consisting of carboxyl, aminocarbonyl, alkylsulfonylaminocarbonyl and alkoxycarbonyl;

R³ is selected from the group consisting of haloalkyl, alkyl, aralkyl, cycloalkyl and aryl optionally substituted with one or more radicals selected from alkylthio, nitro and alkylsulfonyl; and

each R⁴ is independently selected from the group consisting of H, halo, alkyl, aralkyl, alkoxy, aryloxy, heteroaryloxy, aralkyloxy, heteroaralkyloxy, haloalkyl, haloalkoxy, alkylamino, arylamino, aralkylamino, heteroarylamino, heteroarylalkylamino, nitro, amino, aminosulfonyl, alkylaminosulfonyl, arylaminosulfonyl, heteroarylaminosulfonyl, aralkylaminosulfonyl, heteroaralkylaminosulfonyl, heterocyclosulfonyl, alkylsulfonyl, hydroxyarylcarbonyl, nitroaryl, optionally substituted aryl, optionally substituted heteroaryl, aralkylcarbonyl, heteroarylcarbonyl, arylcarbonyl, aminocarbonyl, and alkylcarbonyl;

or R⁴ together with the carbon atoms to which it is attached and the remainder of ring E forms a naphthyl radical.

The cyclooxygenase-2 selective inhibitor may also be a compound of Formula (I) or an isomer, a pharmaceutically acceptable salt, ester, or prodrug thereof wherein:

n is an integer which is 0, 1, 2, 3 or 4;

G is O, S or NR^{a};

R¹ is H;

R^{a} is alkyl;

R² is selected from the group consisting of carboxyl, aminocarbonyl, alkylsulfonylaminocarbonyl and alkoxycarbonyl;

R³ is selected from the group consisting of haloalkyl, alkyl, aralkyl, cycloalkyl and aryl, wherein haloalkyl, alkyl, aralkyl, cycloalkyl, and aryl each is independently optionally substituted with one or more radicals selected from the group consisting of alkylthio, nitro and alkylsulfonyl; and

each R⁴ is independently selected from the group consisting of hydrido, halo, alkyl, aralkyl, alkoxy, aryloxy, heteroaryloxy, aralkyloxy, heteroaralkyloxy, haloalkyl, haloalkoxy, alkylamino, arylamino, aralkylamino, heteroarylamino, heteroarylalkylamino, nitro, amino, aminosulfonyl, alkylaminosulfonyl, arylaminosulfonyl, heteroarylaminosulfonyl, aralkylaminosulfonyl, heteroaralkylaminosulfonyl, heterocyclosulfonyl, alkylsulfonyl, optionally substituted aryl, optionally substituted heteroaryl, aralkylcarbonyl, heteroarylcarbonyl, arylcarbonyl, aminocarbonyl, and alkylcarbonyl; or wherein R⁴ together with ring E forms a naphthyl radical.

In a further embodiment, the cyclooxygenase-2 selective inhibitor may also be a compound of Formula (I) or an isomer, a pharmaceutically acceptable salt, ester, or prodrug thereof, wherein:

n is an integer which is 0, 1, 2, 3 or 4;

G is oxygen or sulfur;

R¹ is H;

R² is carboxyl, lower alkyl, lower aralkyl or lower alkoxycarbonyl;

R³ is lower haloalkyl, lower cycloalkyl or phenyl; and

each R⁴ is H, halo, lower alkyl, lower alkoxy, lower haloalkyl, lower haloalkoxy, lower alkylamino, nitro, amino, aminosulfonyl, lower alkylaminosulfonyl, 5-membered heteroarylalkylaminosulfonyl, 6-membered heteroarylalkylaminosulfonyl, lower aralkylaminosulfonyl, 5-membered nitrogen-containing heterocyclosulfonyl, 6-membered-nitrogen containing heterocyclosulfonyl, lower alkylsulfonyl, optionally substituted phenyl, lower aralkylcarbonyl, or lower alkylcarbonyl; or

R⁴ together with the carbon atoms to which it is attached and the remainder of ring E forms a naphthyl radical.

The cyclooxygenase-2 selective inhibitor may also be a compound of Formula (I) or an isomer, a pharmaceutically acceptable salt, ester, or prodrug thereof wherein:

R² is carboxyl;

R³ is lower haloalkyl; and

each R⁴ is H, halo, lower alkyl, lower haloalkyl, lower haloalkoxy, lower alkylamino, amino, aminosulfonyl, lower alkylaminosulfonyl, 5-membered heteroarylalkylaminosulfonyl, 6-membered heteroarylalkylaminosulfonyl, lower aralkylaminosulfonyl, lower alkylsulfonyl, 6-membered nitrogen-containing heterocyclosulfonyl, optionally substituted phenyl, lower aralkylcarbonyl, or lower alkylcarbonyl; or wherein R⁴ together with ring E forms a naphthyl radical.

The cyclooxygenase-2 selective inhibitor may also be a compound of Formula (I) or an isomer, a pharmaceutically acceptable salt, ester, or prodrug thereof wherein:

n is an integer which is 0, 1, 2, 3 or 4;

R³ is fluoromethyl, chloromethyl, dichloromethyl, trichloromethyl, pentafluoroethyl, heptafluoropropyl, difluoroethyl, difluoropropyl, dichloroethyl, dichloropropyl, difluoromethyl, or trifluoromethyl; and

each R⁴ is H, chloro, fluoro, bromo, iodo, methyl, ethyl, isopropyl, tert-butyl, butyl, isobutyl, pentyl, hexyl, methoxy, ethoxy, isopropyloxy, tertbutyloxy, trifluoromethyl, difluoromethyl, trifluoromethoxy, amino, N,N-dimethylamino, N,N-diethylamino, N-phenylmethylaminosulfonyl, N-phenylethylaminosulfonyl, N-(2-furylmethyl)aminosulfonyl, nitro, N,N-dimethylaminosulfonyl, aminosulfonyl, N-methylaminosulfonyl, N-ethylsulfonyl, 2,2-dimethylethylaminosulfonyl, N,N-dimethylaminosulfonyl, N-(2-methylpropyl)aminosulfonyl, N-morpholinosulfonyl, methylsulfonyl, benzylcarbonyl, 2,2-dimethylpropylcarbonyl, phenylacetyl or phenyl; or wherein R⁴ together with the carbon atoms to which it is attached and the remainder of ring E forms a naphthyl radical.

The cyclooxygenase-2 selective inhibitor may also be a compound of Formula (I) or an isomer, a pharmaceutically acceptable salt, ester, or prodrug thereof wherein:

n is an integer which is 0, 1, 2, 3 or 4;

R³ is trifluoromethyl or pentafluoroethyl; and

each R⁴ is independently H, chloro, fluoro, bromo, iodo, methyl, ethyl, isopropyl, tert-butyl, methoxy, trifluoromethyl, trifluoromethoxy, N-phenylmethylaminosulfonyl, N-phenylethylaminosulfonyl, N-(2-furylmethyl)aminosulfonyl, N,N-dimethylaminosulfonyl, N-methylaminosulfonyl, N-(2,2-dimethylethyl)aminosulfonyl, dimethylaminosulfonyl, 2-methylpropylaminosulfonyl, N-morpholinosulfonyl, methylsulfonyl, benzylcarbonyl, or phenyl; or wherein R⁴ together with the carbon atoms to which it is attached and the remainder of ring E forms a naphthyl radical.

In yet another embodiment, the cyclooxygenase-2 selective inhibitor used in connection with the method(s) of the present invention can also be a compound having the structure of Formula (1) or an isomer, a pharmaceutically acceptable salt, ester, or prodrug thereof:

wherein:

n=4;

G is O or S;

R¹ is H;

R² is CO₂H;

R³ is lower haloalkyl;

a first R⁴ corresponding to R⁹ is hydrido or halo;

a second R⁴ corresponding to R¹⁰ is H, halo, lower alkyl, lower haloalkoxy, lower alkoxy, lower aralkylcarbonyl, lower dialkylaminosulfonyl, lower alkylaminosulfonyl, lower aralkylaminosulfonyl, lower heteroaralkylaminosulfonyl, 5-membered nitrogen-containing heterocyclosulfonyl, or 6-membered nitrogen-containing heterocyclosulfonyl;

a third R⁴ corresponding to R¹¹ is H, lower alkyl, halo, lower alkoxy, or aryl; and

a fourth R⁴ corresponding to R1² is H, halo, lower alkyl, lower alkoxy, and aryl; wherein Formula (I) is represented by Formula (Ia):

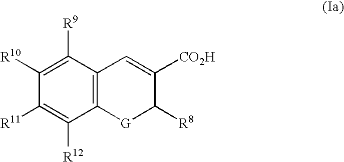

The cyclooxygenase-2 selective inhibitor used in connection with the method(s) of the present invention can also be a compound of having the structure of Formula (Ia) or an isomer, a pharmaceutically acceptable salt, ester, or prodrug thereof wherein:

R⁸ is trifluoromethyl or pentafluoroethyl;

R⁹ is H, chloro, or fluoro;

R¹⁰ is H, chloro, bromo, fluoro, iodo, methyl, tert-butyl, trifluoromethoxy, methoxy, benzylcarbonyl, dimethylaminosulfonyl, isopropylaminosulfonyl, methylaminosulfonyl, benzylaminosulfonyl, phenylethylaminosulfonyl, methylpropylaminosulfonyl, methylsulfonyl, or morpholinosulfonyl;

R¹¹ is H, methyl, ethyl, isopropyl, tert-butyl, chloro, methoxy, diethylamino, or phenyl; and

R¹² is H, chloro, bromo, fluoro, methyl, ethyl, tert-butyl, methoxy, or phenyl.

Examples of exemplary chromene cyclooxygenase-2 selective inhibitors are depicted in Table 1x below.

TABLE 1x
Examples of Chromene Cyclooxygenase-2 Selective Inhibitors as
Embodiments

| Compound | |
|---|---|
| Number | Structural Formula |
| B-3 | 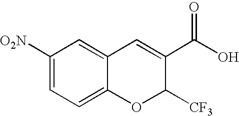 |
| | 6-Nitro-2-trifluoromethyl-2H-1 |
| | -benzopyran-3-carboxylic acid |
| B-4 | 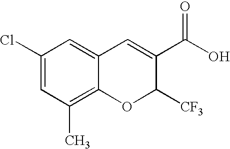 |
| | 6-Chloro-8-methyl-2-trifluoromethyl |
| | 2-H-1-benzopyran-3-carboxylic acid |
| B-5 | 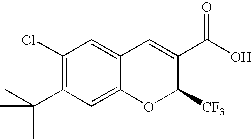 |
| | ((S)-6-Chloro-7-(1,1-dimethylethyl)-2-(trifluo |
| | romethyl-2H-1-benzopyran-3-carboxylic acid |
| B-6 | 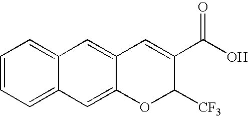 |
| | 2-Trifluoromethyl-2H-naphtho[2,3-b] |
| | pyran-3-carboxylic acid |
| B-7 | 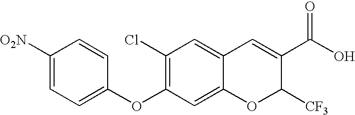 |
| | 6-Chloro-7-(4-nitrophenoxy)-2-(trifluoromethyl)-2H-1- |
| | benzopyran-3-carboxylic acid |
| B-8 | 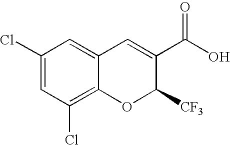 |
| | ((S)-6,8-Dichloro-2-(trifluoromethyl)- |
| | 2H-1-benzopyran-3-carboxylic acid |
| B-9 | 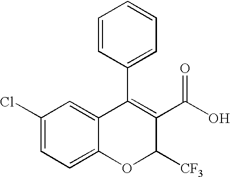 |
| | 6-Chloro-2-(trifluoromethyl)-4-phenyl-2H- |
| | 1-benzopyran-3-carboxylic acid |
| B-10 | 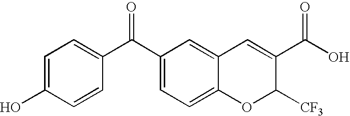 |
| | 6-(4-Hydroxybenzoyl)-2-(trifluoromethyl) |
| | 2-H-1-benzopyran-3-carboxylic acid |
| B-11 | 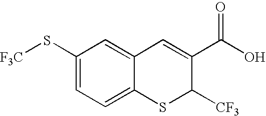 |
| | 2-(Trifluoromethyl)-56-[(trifluoromethyl)thio] |
| | -2H-1-benzothiopyran-3-carboxylic acid |
| B-12 | 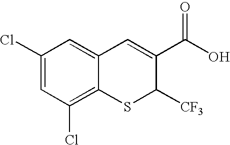 |
| | 6,8-Dichloro-2-trifluoromethyl-2H-1- |
| | benzothiopyran-3-carboxylic acid |
| B-13 | 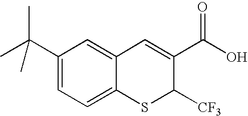 |
| | 6-(1,1-Dimethylethyl)-2-(trifluoromethyl) |
| | -2H-1-benzothiopyran-3-carboxylic acid |
| B-14 | 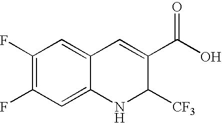 |
| | 6,7-Dichloro-1,2-dihydro-2-(trifluoro |
| | methyl)-3-quinolinecarboxylic acid |
| B-15 | 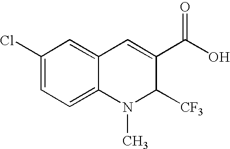 |
| | 6-Chloro-1,2-dihydro-1-methyl-2-(trifluoro |
| | methyl)-3-quinolinecarboxylic acid |
| B-16 | 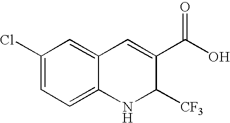 |
| | 6-Chloro-2-(trifluoromethyl)-1,2-dihydro |
| | [1,8]naphthyridine-3-carboxylic acid |
| B-17 | 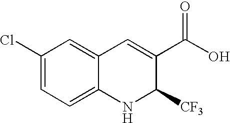 |
| | ((S)-6-Chloro-1,2-dihydro-2-(trifluoro |
| | methyl)-3-quinolinecarboxylic acid |

In a further embodiment, the cyclooxygenase-2 selective inhibitor is selected from the class of tricyclic cyclooxygenase-2 selective inhibitors represented by the general structure of Formula II or an isomer, a pharmaceutically acceptable salt, ester, or prodrug thereof

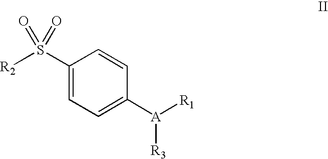

wherein:

A is selected from the group consisting of partially unsaturated or unsaturated heterocyclyl and partially unsaturated or unsaturated carbocyclic rings;

R¹ is selected from the group consisting of heterocyclyl, cycloalkyl, cycloalkenyl and aryl, wherein R¹ is optionally substituted at a substitutable position with one or more radicals selected from alkyl, haloalkyl, cyano, carboxyl, alkoxycarbonyl, hydroxyl, hydroxyalkyl, haloalkoxy, amino, alkylamino, arylamino, nitro, alkoxyalkyl, alkylsulfinyl, halo, alkoxy and alkylthio;

R² is selected from the group consisting of methyl or amino; and

R³ is selected from the group consisting of a radical selected from H, halo, alkyl, alkenyl, alkynyl, oxo, cyano, carboxyl, cyanoalkyl, heterocyclyloxy, alkyloxy, alkylthio, alkylcarbonyl, cycloalkyl, aryl, haloalkyl, heterocyclyl, cycloalkenyl, aralkyl, heterocyclylalkyl, acyl, alkylthioalkyl, hydroxyalkyl, alkoxycarbonyl, arylcarbonyl, aralkylcarbonyl, aralkenyl, alkoxyalkyl, arylthioalkyl, aryloxyalkyl, aralkylthioalkyl, aralkoxyalkyl, alkoxyaralkoxyalkyl, alkoxycarbonylalkyl, aminocarbonyl, aminocarbonylalkyl, alkylaminocarbonyl, N-arylaminocarbonyl, N-alkyl-N-arylaminocarbonyl, alkylaminocarbonylalkyl, carboxyalkyl, alkylamino, N-arylamino, N-aralkylamino, N-alkyl-N-aralkylamino, N-alkyl-N-arylamino, aminoalkyl, alkylaminoalkyl, N-arylaminoalkyl, N-aralkylaminoalkyl, N-alkyl-N-aralkylaminoalkyl, N-alkyl-N-arylaminoalkyl, aryloxy, aralkoxy, arylthio, aralkylthio, alkylsulfinyl, alkylsulfonyl, aminosulfonyl, alkylaminosulfonyl, N-arylaminosulfonyl, arylsulfonyl, N-alkyl-N-arylaminosulfonyl.

In another embodiment, the cyclooxygenase-2 selective inhibitor represented by the above Formula II is selected from the group of compounds illustrated in Table 2x, consisting of celecoxib (B-18; U.S. Pat. No. 5,466,823; CAS No. 169590-42-5), valdecoxib (B-19; U.S. Pat. No. 5,633,272; CAS No. 181695-72-7), deracoxib (B-20; U.S. Pat. No. 5,521,207; CAS No. 169590-41-4), rofecoxib (B-21; CAS No. 162011-90-7), etoricoxib (MK-663; B-22; PCT publication WO 98/03484), tilmacoxib (JTE-522; B-23; CAS No. 180200-68-4).

TABLE 2x
Examples of Tricyclic Cyclooxygenase-2 Selective Inhibitors as
Embodiments

| Compound | |
|---|---|
| Number | Structural Formula |
| B-18 | 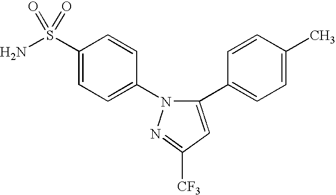 |
| B-19 | 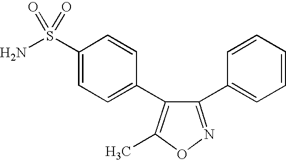 |
| B-20 | 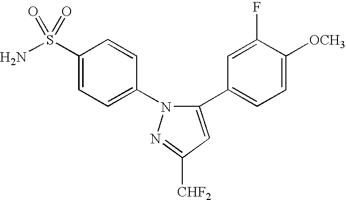 |
| B-21 | 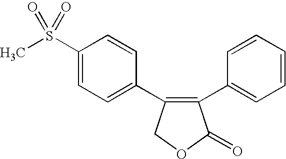 |
| B-22 | 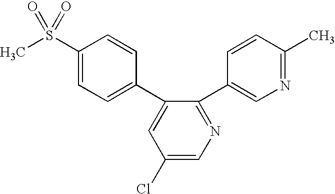 |
| B-23 | 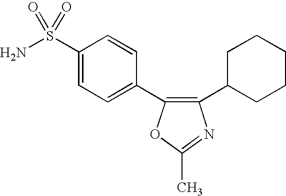 |

In still another embodiment, the cyclooxygenase-2 selective inhibitor is selected from the group consisting of celecoxib, rofecoxib and etoricoxib.

In yet another embodiment, the cyclooxygenase-2 selective inhibitor is parecoxib (B-24, U.S. Pat. No. 5,932,598, CAS No. 198470-84-7), which is a therapeutically effective prodrug of the tricyclic cyclooxygenase-2 selective inhibitor valdecoxib, B-1 9, may be advantageously employed as a source of a cyclooxygenase inhibitor (U.S. Pat. No. 5,932,598, herein incorporated by reference).

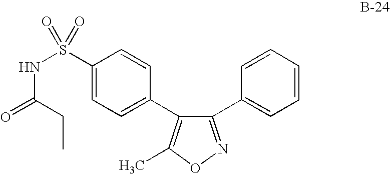

One form of parecoxib is sodium parecoxib.

In another embodiment of the invention, the compound having the formula B-25 or an isomer, a pharmaceutically acceptable salt, ester, or prodrug of a compound having formula B-25 that has been previously described in International Publication number WO 00/24719 (which is herein incorporated by reference) is another tricyclic cyclooxygenase-2 selective inhibitor that may be advantageously employed.

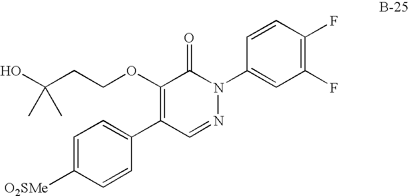

Another cyclooxygenase-2 selective inhibitor that is useful in connection with the method(s) of the present invention is N-(2-cyclohexyloxynitrophenyl)-methane sulfonamide (NS-398) having a structure shown below as B-26, or an isomer, a pharmaceutically acceptable salt, ester, or prodrug of a compound having formula B-26.

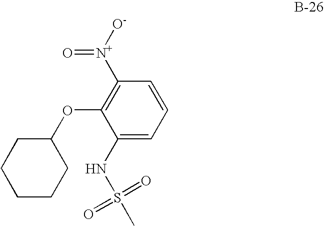

In yet a further embodiment, the cyclooxygenase-2 selective inhibitor thereof used in connection with the method(s) of the present invention can be selected from the class of phenylacetic acid derivative cyclooxygenase-2 selective inhibitors represented by the general structure of Formula (III) or an isomer, a pharmaceutically acceptable salt, ester, or prodrug:

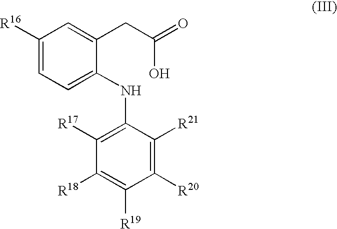

wherein

R¹⁶ is methyl or ethyl;

R¹⁷ is chloro or fluoro;

R¹⁸ is hydrogen or fluoro;

R¹⁹ is hydrogen, fluoro, chloro, methyl, ethyl, methoxy, ethoxy or hydroxy;

R²⁰ is hydrogen or fluoro; and

R²¹ is chloro, fluoro, trifluoromethyl or methyl,

provided that R¹⁷, R¹⁸, R¹⁹ and R²⁰ are not all fluoro when R¹⁶ is ethyl and R¹⁹ is H.

Another phenylacetic acid derivative cyclooxygenase-2 selective inhibitor used in connection with the method(s) of the present invention is a compound that has the designation of COX 189 (lumiracoxib; B-2 11) and that has the structure shown in Formula (III) or an isomer, a pharmaceutically acceptable salt, ester, or prodrug thereof wherein:

R¹⁶ is ethyl;

R¹⁷ and R¹⁹ are chloro;

R¹⁸ and R²⁰ are hydrogen; and

and R²¹ is methyl.

In yet another embodiment, the cyclooxygenase-2 selective inhibitor is represented by Formula (IV) or an isomer, a pharmaceutically acceptable salt, ester, or prodrug thereof:

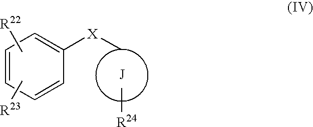

wherein:

X is O or S;

J is a carbocycle or a heterocycle;

R²² is NHSO₂CH₃ or F;

R²³ is H, NO₂, or F; and

R24 is H, NHSO₂CH₃, or (SO₂CH₃)C₆H₄.

According to another embodiment, the cyclooxygenase-2 selective inhibitors used in the present method(s) have the structural Formula (V) or an isomer, a pharmaceutically acceptable salt, ester, or prodrug thereof:

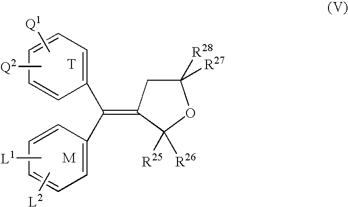

wherein:

T and M independently are phenyl, naphthyl, a radical derived from a heterocycle comprising 5 to 6 members and possessing from 1 to 4 heteroatoms, or a radical derived from a saturated hydrocarbon ring having from 3 to 7 carbon atoms;

Q¹, Q², L¹ or L² are independently hydrogen, halogen, lower alkyl having from 1 to 6 carbon atoms, trifluoromethyl, or lower methoxy having from 1 to 6 carbon atoms; and

at least one of Q¹, Q², L¹ or L² is in the para position and is —S(O)ₙ—R, wherein n is 0, 1, or 2 and R is a lower alkyl radical having 1 to 6 carbon atoms or a lower haloalkyl radical having from 1 to 6 carbon atoms, or an —SO₂NH_{2;} or,

Q¹ and Q² are methylenedioxy; or

L¹ and L² are methylenedioxy; and

R²⁵, R²⁶, R²⁷, and R²⁸ are independently hydrogen, halogen, lower alkyl radical having from 1 to 6 carbon atoms, lower haloalkyl radical having from 1 to 6 carbon atoms, or an aromatic radical selected from the group consisting of phenyl, naphthyl, thienyl, furyl and pyridyl; or,

R²⁵ and R²⁶ are O; or,

R²⁷ and R²⁸ are O; or,

R²⁵, R²⁶, together with the carbon atom to which they are attached, form a saturated hydrocarbon ring having from 3 to 7 carbon atoms; or,

R²⁷, R²⁸, together with the carbon atom to which they are attached, form a saturated hydrocarbon ring having from 3 to 7 carbon atoms.

In another embodiment, the compounds N-(2-cyclohexyloxynitrophenyl)methane sulfonamide, and (E)-4-[(4-methylphenyl)(tetrahydro-2-oxo-3-furanylidene) methyl]benzenesulfonamide or an isomer, a pharmaceutically acceptable salt, ester, or prodrug thereof having the structure of Formula (V) are employed as cyclooxygenase-2 selective inhibitors.

In a further embodiment, compounds that are useful for the cyclooxygenase-2 selective inhibitor or an isomer, a pharmaceutically acceptable salt, ester, or prodrug thereof used in connection with the method(s) of the present invention, the structures for which are set forth in Table 3x below, include, but are not limited to:

6-chloro-2-trifluoromethyl-2H-1-benzopyran-3-carboxylic acid (B-27);

6-chloro-7-methyl-2-trifluoromethyl-2H-1-benzopyran-3-carboxylic acid (B-28);

8-(1-methylethyl)-2-trifluoromethyl-2H-1-benzopyran-3-carboxylic acid (B-29);

6-chloro-8-(1-methylethyl)-2-trifluoromethyl-2H-1-benzopyran-3-carboxylic acid (B-30);

2-trifluoromethyl-3H-naphtho[2,1-b]pyran-3-carboxylic acid (B-31);

7-(1,1-dimethylethyl)-2-trifluoromethyl-2H-1-benzopyran-3-carboxylic acid (B-32);

6-bromo-2-trifluoromethyl-2H-1-benzopyran-3-carboxylic acid (B-33);

8-chloro-2-trifluoromethyl-2H-1-benzopyran-3-carboxylic acid (B-34);

6-trifluoromethoxy-2-trifluoromethyl-2H-1-benzopyran-3-carboxylic acid (B-35);

5,7-dichloro-2-trifluoromethyl-2H-1-benzopyran-3-carboxylic acid (B-36);

8-phenyl-2-trifluoromethyl-2H-1-benzopyran-3-carboxylic acid (B-37);

7,8-dimethyl-2-trifluoromethyl-2H-1-benzopyran-3-carboxylic acid (B-38);

6,8-bis(dimethylethyl)-2-trifluoromethyl-2H-1-benzopyran-3-carboxylic acid (B-39);

7-(1-methylethyl)-2-trifluoromethyl-2H-1-benzopyran-3-carboxylic acid (B-40);

7-phenyl-2-trifluoromethyl-2H-1-benzopyran-3-carboxylic acid (B-41);

6-chloro-7-ethyl-2-trifluoromethyl-2H-1-benzopyran-3-carboxylic acid (B-42);

6-chloro-8-ethyl-2-trifluoromethyl-2H-1-benzopyran-3-carboxylic acid (B-43);

6-chloro-7-phenyl-2-trifluoromethyl-2H-1-benzopyran-3-carboxylic acid (B-44);

6,7-dichloro-2-trifluoromethyl-2H-1-benzopyran-3-carboxylic acid (B-45);

6,8-dichloro-2-trifluoromethyl-2H-1-benzopyran-3-carboxylic acid (B-46);

6-chloro-8-methyl-2-trifluoromethyl-2H-1-benzopyran-3-carboxylic acid (B-47);

8-chloro-6-methyl-2-trifluoromethyl-2H-1-benzopyran-3-carboxylic acid (B-48)

8-chloro-6-methoxy-2-trifluoromethyl-2H-1-benzopyran-3-carboxylic acid (B-49);

6-bromo-8-chloro-2-trifluoromethyl-2H-1-benzopyran-3-carboxylic acid (B-50);

8-bromo-6-fluoro-2-trifluoromethyl-2H-1-benzopyran-3-carboxylic acid (B-51);

8-bromo-6-methyl-2-trifluoromethyl-2H-1-benzopyran-3-carboxylic acid (B-52);

8-bromo-5-fluoro-2-trifluoromethyl-2H-1-benzopyran-3-carboxylic acid (B-53);

6-chloro-8-fluoro-2-trifluoromethyl-2H-1-benzopyran-3-carboxylic acid (B-54);

6-bromo-8-methoxy-2-trifluoromethyl-2H-1-benzopyran-3-carboxylic acid (B-55);

6-[[(phenylmethyl)amino]sulfonyl]-2-trifluoromethyl-2H-1-benzopyran-3-carboxylic acid (B-56);

6-[(dimethylamino)sulfonyl]-2-trifluoromethyl-2H-1-benzopyran-3-carboxylic acid (B-57);

6-[(methylamino)sulfonyl]-2-trifluoromethyl-2H-1-benzopyran-3-carboxylic acid (B-58);

6-[(4-morpholino)sulfonyl]-2-trifluoromethyl-2H-1-benzopyran-3-carboxylic acid (B-59);

6-[(1,1-dimethylethyl)aminosulfonyl]-2-trifluoromethyl-2H-1-benzopyran-3-carboxylic acid (B-60);

6-[(2-methylpropyl)aminosulfonyl]-2-trifluoromethyl-2H-1-benzopyran-3-carboxylic acid (B-61);

6-methylsulfonyl-2-trifluoromethyl-2H-1-benzopyran-3-carboxylic acid (B-62);

8-chloro-6-[[(phenylmethyl)amino]sulfonyl]-2-trifluoromethyl-2H-1-benzopyran-3-carboxylic acid (B-63);

6-phenylacetyl-2-trifluoromethyl-2H-1-benzopyran-3-carboxylic acid (B-64);

6,8-dibromo-2-trifluoromethyl-2H-1-benzopyran-3-carboxylic acid (B-65);

8-chloro-5,6-dimethyl-2-trifluoromethyl-2H-1-benzopyran-3-carboxylic acid (B-66);

6,8-dichloro-(S)-2-trifluoromethyl-2H-1-benzopyran-3-carboxylic acid (B-67);

6-benzylsulfonyl-2-trifluoromethyl-2H-1-benzopyran-3-carboxylic acid (B-68);

6-[[N-(2-furylmethyl)amino]sulfonyl]-2-trifluoromethyl-2H-1-benzopyran-3-carboxylic acid (B-69);

6-[[N-(2-phenylethyl)amino]sulfonyl]-2-trifluoromethyl-2H-1-benzopyran-3-carboxylic acid (B-70);

6-iodo-2-trifluoromethyl-2H-1-benzopyran-3-carboxylic acid (B-71);

7-(1,1-dimethylethyl)-2-pentafluoroethyl-2H-1-benzopyran-3-carboxylic acid (B-72);

6-chloro-2-trifluoromethyl-2H-1-benzothiopyran-3-carboxylic acid (B-73);

3-[(3-Chloro-phenyl)-(4-methanesulfonyl-phenyl)-methylene]-dihydro-furan-2-one or BMS-347070(B-74);

8-acetyl-3-(4-fluorophenyl)-2-(4-methylsulfonyl)phenyl-imidazo(1,2-a)pyridine (B-75);

5,5-dimethyl-4-(4-methylsulfonyl)phenyl-3-phenyl-2-(5H)-furanone (B-76);

5-(4-fluorophenyl)-1-[4-(methylsulfonyl)phenyl]-3-(trifluoromethyl)pyrazole (B-77);

4-(4-fluorophenyl)-5-[4-(methylsulfonyl)phenyl]-1-phenyl-3-(trifluoromethyl)pyrazole (B-78);

4-(5-(4-chlorophenyl)-3-(4-methoxyphenyl)-1H-pyrazol-1-yl)benzenesulfonamide (B-79);

4-(3,5-bis(4-methylphenyl)-1H-pyrazol-1-yl)benzenesulfonamide (B-80);

4-(5-(4-chlorophenyl)-3-phenyl-1H-pyrazol-1-yl)benzenesulfonamide (B-81);

4-(3,5-bis(4-methoxyphenyl)-1H-pyrazol-1-yl)benzenesulfonamide (B-82);

4-(5-(4-chlorophenyl)-3-(4-methylphenyl)-1H-pyrazol-1-yl)benzenesulfonamide (B-83);

4-(5-(4-chlorophenyl)-3-(4-nitrophenyl)-1H-pyrazol-1-yl)benzenesulfonamide (B-84);

4-(5-(4-chlorophenyl)-3-(5-chloro-2-thienyl)-1H-pyrazol-1-yl)benzenesulfonamide (B-85);

4-(4-chloro-3,5-diphenyl-1H-pyrazol-1-yl)benzenesulfonamide (B-86);

4-[5-(4-chlorophenyl)-3-(trifluoromethyl)-1H-pyrazol-1-yl]benzenesulfonamide (B-87);

4-[5-phenyl-3-(trifluoromethyl)-1H-pyrazol-1-yl]benzenesulfonanide (B-88);

4-[5-(4-fluorophenyl)-3-(trifluoromethyl)-1H-pyrazol-1-yl]benzenesulfonamide (B-89);

4-[5-(4-methoxyphenyl)-3-(trifluoromethyl)-1H-pyrazol-1-yl]benzenesulfonamide (B-90);

4-[5-(4-chlorophenyl)-3-(difluoromethyl)-1H-pyrazol-1-yl]benzenesulfonamide (B-91);

4-[5-(4-methylphenyl)-3-(trifluoromethyl)-1H-pyrazol-1-yl]benzenesulfonamide (B-92);

4-[4-chloro-5-(4-chlorophenyl)-3-(trifluoromethyl)-1H-pyrazol-1-yl]benzenesulfonamide (B-93);

4-[3-(difluoromethyl)-5-(4-methylphenyl)-1H-pyrazol-1-yl]benzenesulfonamide (B-94);

4-[3-(difluoromethyl)-5-phenyl-1H-pyrazol-1-yl]benzenesulfonamide (B-95);

4-[3-(difluoromethyl)-5-(4-methoxyphenyl)-1H-pyrazol-1-yl]benzenesulfonamide (B-96);

4-[3-cyano-5-(4-fluorophenyl)-1H-pyrazol-1-yl]benzenesulfonamide (B-97);

4-[3-(difluoromethyl)-5-(3-fluoro-4-methoxyphenyl)-1H-pyrazol-1-yl]benzenesulfonamide (B-98);

4-[5-(3-fluoro-4-methoxyphenyl)-3-(trifluoromethyl)-1H-pyrazol-1-yl]benzenesulfonamide (B-99);

4-[4-chloro-5-phenyl-1H-pyrazol-1-yl]benzenesulfonamide (B-100);

4-[5-(4-chlorophenyl)-3-(hydroxymethyl)-1H-pyrazol-1-yl]benzenesulfonamide (B-101);

4-[5-(4-(N,N-dimethylamino)phenyl)-3-(trifluoromethyl)-1H-pyrazol-1-yl]benzenesulfonamide (B-102);

5-(4-fluorophenyl)-6-[4-(methylsulfonyl)phenyl]spiro[2.4]hept-5-ene (B-103);

4-[6-(4-fluorophenyl)spiro[2.4]hept-5-en-5-yl]benzenesulfonamide (B-104);

6-(4-fluorophenyl)-7-[4-(methylsulfonyl)phenyl]spiro[3.4]oct-6-ene (B-105);

5-(3-chloro-4-methoxyphenyl)-6-[4-(methylsulfonyl)phenyl]spiro[2.4]hept-5-ene (B-106);

4-[6-(3-chloro-4-methoxyphenyl)spiro[2.4]hept-5-en-5-yl]benzenesulfonamide (B-107);

5-(3,5-dichloro-4-methoxyphenyl)-6-[4-(methylsulfonyl)phenyl]spiro[2.4]hept-5-ene (B-108);

5-(3-chloro-4-fluorophenyl)-6-[4-(methylsulfonyl)phenyl]spiro[2.4]hept-5-ene (B-109);

4-[6-(3,4-dichlorophenyl)spiro[2.4]hept-5-en-5-yl]benzenesulfonamide (B-110);

2-(3-chloro-4-fluorophenyl)-4-(4-fluorophenyl)-5-(4-methylsulfonylphenyl)thiazole (B-111);

2-(2-chlorophenyl)-4-(4-fluorophenyl)-5-(4-methylsulfonylphenyl)thiazole (B-112);

5-(4-fluorophenyl)-4-(4-methylsulfonylphenyl)-2-methylthiazole (B-113);

4-(4-fluorophenyl)-5-(4-methylsulfonylphenyl)-2-trifluoromethylthiazole (B-114);

4-(4-fluorophenyl)-5-(4-methylsulfonylphenyl)-2-(2-thienyl)thiazole (B-115);

4-(4-fluorophenyl)-5-(4-methylsulfonylphenyl)-2-benzylaminothiazole (B-116);

4-(4-fluorophenyl)-5-(4-methylsulfonylphenyl)-2-(1-propylamino)thiazole (B-117);

2-[(3,5-dichlorophenoxy)methyl)-4-(4-fluorophenyl)-5-[4-(methylsulfonyl)phenyl]thiazole (B-118);

5-(4-fluorophenyl)-4-(4-methylsulfonylphenyl)-2-trifluoromethylthiazole (B-119);

1-methylsulfonyl-4-[1,1-dimethyl-4-(4-fluorophenyl)cyclopenta-2,4-dien-3-yl]benzene (B-120);

4-[4-(4-fluorophenyl)-1,1-dimethylcyclopenta-2,4-dien-3-yl]benzenesulfonamide (B-121);

5-(4-fluorophenyl)-6-[4-(methylsulfonyl)phenyl]spiro[2.4]hepta-4,6-diene (B-122);

4-[6-(4-fluorophenyl)spiro[2.4]hepta-4,6-dien-5-yl]benzenesulfonamide (B-123);

6-(4-fluorophenyl)-2-methoxy-5-[4-(methylsulfonyl)phenyl] -pyridine-3-carbonitrile (B-124);

2-bromo-6-(4-fluorophenyl)-5-[4-(methylsulfonyl)phenyl]-pyridine-3-carbonitrile (B-125);

6-(4-fluorophenyl)-5-[4-(methylsulfonyl)phenyl]-2-phenyl-pyridine-3-carbonitrile (B-126);

4-[2-(4-methylpyridin-2-yl)-4-(trifluoromethyl)-1H-imidazol-1-yl]benzenesulfonamide (B-127);

4-[2-(5-methylpyridin-3-yl)-4-(trifluoromethyl)-1H-imidazol-1-yl]benzenesulfonamide (B-128);

4-[2-(2-methylpyridin-3-yl)-4-(trifluoromethyl)-1H-imidazol-1-yl]benzenesulfonamide (B-129);

3-[1-[4-(methylsulfonyl)phenyl]-4-(trifluoromethyl)-1H-imidazol-2-yl]pyridine (B-130);

2-[1-[4-(methylsulfonyl)phenyl-4-(trifluoromethyl)-1H-imidazol-2-yl]pyridine (B-131);

2-methyl-4-[1-[4-(methylsulfonyl)phenyl-4-(trifluoromethyl)-1H-imidazol-2-yl]pyridine (B-132);

2-methyl-6-[1-[4-(methylsulfonyl)phenyl-4-(trifluoromethyl)-1H-imidazol-2-yl]pyridine (B-133);

4-[2-(6-methylpyridin-3-yl)-4-(trifluoromethyl)-1H-imidazol-1-yl]benzenesulfonamide (B-134);

2-(3,4-difluorophenyl)-1-[4-(methylsulfonyl)phenyl]-4-(trifluoromethyl)-1H-imidazole (B-135);

4-[2-(4-methylphenyl)-4-(trifluoromethyl)-1H-imidazol-1-yl]benzenesulfonamide (B-136);

2-(4-chlorophenyl)-1-[4-(methylsulfonyl)phenyl]-4-methyl-1H-imidazole (B-137);

2-(4-chlorophenyl)-1-[4-(methylsulfonyl)phenyl]-4-phenyl-1H-imidazole (B-138);

2-(4-chlorophenyl)-4-(4-fluorophenyl)-1-[4-(methylsulfonyl)phenyl]-1H-imidazole (B-139);

2-(3-fluoro-4-methoxyphenyl)-1-[4-(methylsulfonyl)phenyl-4-(trifluoromethyl)-1H-imidazole (B-140);

1-[4-(methylsulfonyl)phenyl]-2-phenyl-4-trifluoromethyl-1H-imidazole (B-141);

2-(4-methylphenyl)-1-[4-(methylsulfonyl)phenyl]-4-trifluoromethyl-1H-imidazole (B-142);

4-[2-(3-chloro-4-methylphenyl)-4-(trifluoromethyl)-1H-imidazol-1-yl]benzenesulfonamide (B-143);

2-(3-fluoro-5-methylphenyl)-1-[4-(methylsulfonyl)phenyl]-4-(trifluoromethyl)-1H-imidazole (B-144);

4-[2-(3-fluoro-5-methylphenyl)-4-(trifluoromethyl)-1H-imidazol-1-yl]benzenesulfonamide (B-145);

2-(3-methylphenyl)-1-[4-(methylsulfonyl)phenyl]-4-trifluoromethyl-1H-imidazole (B-146);

4-[2-(3-methylphenyl)-4-trifluoromethyl-1H-imidazol-1-yl]benzenesulfonamide (B-147);

1-[4-(methylsulfonyl)phenyl]-2-(3-chlorophenyl)-4-trifluoromethyl-1H-imidazole (B-148);

4-[2-(3-chlorophenyl)-4-trifluoromethyl-1H-imidazol-1-yl]benzenesulfonamide (B-149);

4-[2-phenyl-4-trifluoromethyl-1H-imidazol-1-yl]benzenesulfonamide (B-150);

4-[2-(4-methoxy-3-chlorophenyl)-4-trifluoromethyl-1H-imidazol-1-yl]benzenesulfonamide (B-151);

1-allyl-4-(4-fluorophenyl)-3-[4-(methylsulfonyl)phenyl]-5-(trifluoromethyl)-1H-pyrazole (B-152);

4-[1-ethyl-4-(4-fluorophenyl)-5-(trifluoromethyl)-1H-pyrazol-3-yl]benzenesulfonamide (B-153);

N-phenyl-[4-(4-fluorophenyl)-3-[4-(methylsulfonyl)phenyl]-5-(trifluoromethyl)-1H-pyrazol-1-yl]acetamide (B-154);

ethyl [4-(4-fluorophenyl)-3-[4-(methylsulfonyl)phenyl]-5-(trifluoromethyl)-1H-pyrazol-1-yl]acetate (B-155);

4-(4-fluorophenyl)-3-[4-(methylsulfonyl)phenyl]-1-(2-phenylethyl)-1H-pyrazole (B-156);

4-(4-fluorophenyl)-3-[4-(methylsulfonyl)phenyl]-1-(2-phenylethyl)-5-(trifluoromethyl)pyrazole (B-157);

1-ethyl-4-(4-fluorophenyl)-3-[4-(methylsulfonyl)phenyl]-5-(trifluoromethyl)-1H-pyrazole (B-158);

5-(4-fluorophenyl)-4-(4-methylsulfonylphenyl)-2-trifluoromethyl-1H-imidazole (B-159);

4-[4-(methylsulfonyl)phenyl]-5-(2-thiophenyl)-2-(trifluoromethyl)-1H-imidazole (B-160);

5-(4-fluorophenyl)-2-methoxy-4-[4-(methylsulfonyl)phenyl]-6-(trifluoromethyl)pyridine (B-161);

2-ethoxy-5-(4-fluorophenyl)-4-[4-(methylsulfonyl)phenyl]-6-(trifluoromethyl)pyridine (B-162);

5-(4-fluorophenyl)-4-[4-(methylsulfonyl)phenyl]-2-(2-propynyloxy)-6-(trifluoromethyl)pyridine (B-163);

2-bromo-5-(4-fluorophenyl)-4-[4-(methylsulfonyl)phenyl]-6-(trifluoromethyl)pyridine (B-164);

4-[2-(3-chloro-4-methoxyphenyl)-4,5-difluorophenyl]benzenesulfonamide (B-165);

1-(4-fluorophenyl)-2-[4-(methylsulfonyl)phenyl]benzene (B-166);

5-difluoromethyl-4-(4-methylsulfonylphenyl)-3-phenylisoxazole (B-167);

4-[3-ethyl-5-phenylisoxazol-4-yl]benzenesulfonamide (B-168);

4-[5-difluoromethyl-3-phenylisoxazol-4-yl]benzenesulfonamide (B-169);

4-[5-hydroxymethyl-3-phenylisoxazol-4-yl]benzenesulfonamide (B-170);

4-[5-methyl-3-phenyl-isoxazol-4-yl]benzenesulfonamide (B-171);

1-[2-(4-fluorophenyl)cyclopenten-1-yl]-4-(methylsulfonyl)benzene (B-172);

1-[2-(4-fluoro-2-methylphenyl)cyclopenten-1-yl]-4-(methylsulfonyl)benzene (B-173);

1-[2-(4-chlorophenyl)cyclopenten-1-yl]-4-(methylsulfonyl)benzene (B-174);

1-[2-(2,4-dichlorophenyl)cyclopenten-1-yl]-4-(methylsulfonyl)benzene (B-175);

1-[2-(4-trifluoromethylphenyl)cyclopenten-1-yl]-4-(methylsulfonyl)benzene (B-176);

1-[2-(4-methylthiophenyl)cyclopenten-1-yl]-4-(methylsulfonyl)benzene (B-177);

1-[2-(4-fluorophenyl)-4,4-dimethylcyclopenten-1-yl]-4-(methylsulfonyl)benzene (B-178);

4-[2-(4-fluorophenyl)-4,4-dimethylcyclopenten-1-yl]benzenesulfonamide (B-179);

1-[2-(4-chlorophenyl)-4,4-dimethylcyclopenten-1-yl]-4-(methylsulfonyl)benzene (B-180);

4-[2-(4-chlorophenyl)-4,4-dimethylcyclopenten-1-yl]benzenesulfonamide (B-181);

4-[2-(4-fluorophenyl)cyclopenten-1-yl]benzenesulfonamide (B-182);

4-[2-(4-chlorophenyl)cyclopenten-1-yl]benzenesulfonamide (B-183);

1-[2-(4-methoxyphenyl)cyclopenten-1-yl]-4-(methylsulfonyl)benzene (B-184);

1-[2-(2,3-difluorophenyl)cyclopenten-1-yl]-4-(methylsulfonyl)benzene (B-185);

4-[2-(3-fluoro-4-methoxyphenyl)cyclopenten-1-yl]benzenesulfonamide (B-186);

1-[2-(3-chloro-4-methoxyphenyl)cyclopenten-1-yl]-4-(methylsulfonyl)benzene (B-187);

4-[2-(3-chloro-4-fluorophenyl)cyclopenten-1-yl]benzenesulfonamide (B-188);

4-[2-(2-methylpyridin-5-yl)cyclopenten-1-yl]benzenesulfonamide (B-189);

ethyl 2-[4-(4-fluorophenyl)-5-[4-(methylsulfonyl) phenyl]oxazol-2-yl]-2-benzyl-acetate (B-190);

2-[4-(4-fluorophenyl)-5-[4-(methylsulfonyl)phenyl]oxazol-2-yl]acetic acid (B-191);

2-(tert-butyl)-4-(4-fluorophenyl)-5-[4-(methylsulfonyl)phenyl]oxazole (B-192);

4-(4-fluorophenyl)-5-[4-(methylsulfonyl)phenyl]-2-phenyloxazole (B-193);

4-(4-fluorophenyl)-2-methyl-5-[4-(methylsulfonyl)phenyl]oxazole (B-194);

4-[5-(3-fluoro-4-methoxyphenyl)-2-trifluoromethyl-4-oxazolyl]benzenesulfonamide (B-195);

6-chloro-7-(1,1-dimethylethyl)-2-trifluoromethyl-2H-1-benzopyran-3-carboxylic acid (B-196);

6-chloro-8-methyl-2-trifluoromethyl-2H-1-benzopyran-3-carboxylic acid (B-197);

5,5-dimethyl-3-(3-fluorophenyl)-4-methylsulfonyl-2(5H)-furanone (B-198);

6-chloro-2-trifluoromethyl-2H-1-benzothiopyran-3-carboxylic acid (B-199);

4-[5-(4-chlorophenyl)-3-(trifluoromethyl)-1H-pyrazol-1-yl]benzenesulfonamide (B-200);

4-[5-(4-methylphenyl)-3-(trifluoromethyl)-1H-pyrazol-1-yl]benzenesulfonamide (B-201);

4-[5-(3-fluoro-4-methoxyphenyl)-3-(difluoromethyl)-1H-pyrazol-1-yl]benzenesulfonamide (B-202);

3-[1-[4-(methylsulfonyl)phenyl]-4-trifluoromethyl-1H-imidazol-2-yl]pyridine (B-203);

2-methyl-5-[1-[4-(methylsulfonyl)phenyl]-4-trifluoromethyl-1H-imidazol-2-yl]pyridine (B-204);

4-[2-(5-methylpyridin-3-yl)-4-(trifluoromethyl)-1H-imidazol-1-yl]benzenesulfonamide (B-205);

4-[5-methyl-3-phenylisoxazol-4-yl]benzenesulfonamide (B-206);

4-[5-hydroxymethyl-3-phenylisoxazol-4-yl]benzenesulfonamide (B-207);

[2-trifluoromethyl-5-(3,4-difluorophenyl)-4-oxazolyl]benzenesulfonamide (B-208);

4-[2-methyl-4-phenyl-5-oxazolyl]benzenesulfonamide (B-209);

4-[5-(2-fluoro-4-methoxyphenyl)-2-trifluoromethyl-4-oxazolyl]benzenesulfonamide (B-210);

[2-(2-chloro-6-fluoro-phenylamino)-5-methyl-phenyl]-acetic acid or COX 189(lumiracoxib; B-21 1);

N-(4-Nitro-2-phenoxy-phenyl)-methanesulfonamide or nimesulide (B-212);

N-[6-(2,4-difluoro-phenoxy)-1-oxo-indan-5-yl]-methanesulfonamide or flosulide (B-213);

N-[6-(2,4-Difluoro-phenylsulfanyl)-1-oxo-1H-inden-5-yl]-methanesulfonamide, soldium salt or L-745337(B-214);

N-[5-(4-fluoro-phenylsulfanyl)-thiophen-2-yl]-methanesulfonamide or RWJ-63556 (B-215);

3-(3,4-Difluoro-phenoxy)-4-(4-methanesulfonyl-phenyl)-5-methyl-5-(2,2,2-trifluoro-ethyl)-5H-furan-2-one or L-784512or L-784512(B-216);

(5Z)-2-amino-5-[[3,5-bis(1,1-dimethylethyl)-4-hydroxyphenyl]methylene]-4(5H)-thiazolone or darbufelone (B-217);

CS-502(B-218);

LAS-34475(B-219);

LAS-34555(B-220);

S-33516(B-221);

SD-8381(B-222);

L-783003(B-223);

N-[3-(formylamino)-4-oxo-6-phenoxy-4H-1-benzopyran-7-yl]-methanesulfonamide or T-614(B-224);

D-1367(B-225);

L-748731(B-226);

(6aR,10aR)-3-(1,1-dimethylheptyl)-6a,7,10,10a-tetrahydro-1-hydroxy-6,6-dimethyl-6H-dibenzo[b,d]pyran-9-carboxylic acid or CT3(B-227);

CGP-28238(B-228);

4-[[3,5-bis(1,1-dimethylethyl)-4-hydroxyphenyl]methylene]dihydro-2-methyl-2H-1,2-oxazin-3(4H)-one or BF-389(B-229);

GR-253035(B-230);

6-dioxo-9H-purin-8-yl-cinnamic acid (B-231);

S-2474(B-232);

4-[4-(methyl)-sulfonyl)phenyl]-3-phenyl-2(5H)-furanone;

4-(5-methyl-3-phenyl-4-isoxazolyl);

2-(6-methylpyrid-3-yl)-3-(4-methylsulfonylphenyl)-5-chloropyridine;

4-[5-(4-methylphenyl)-3-(trifluoromethyl)-1H-pyrazol-1-yl];

N-[[4-(5-methyl-3-phenyl-4-isoxazolyl)phenyl]sulfonyl];

4-[5-(3-fluoro-4-methoxyphenyl)-3-difluoromethyl)-1H-pyrazol-1-yl]benzenesulfonamide;

(S)-6,8-dichloro-2-(trifluoromethyl)-2H-1-benzopyran-3-carboxylic acid;

2-(3,4-difluorophenyl)-4-(3-hydroxy-3-methylbutoxy)-5-[4-(methysulfonyl)phenyl]-3(2H)-pyridzainone;

2-trifluoromethyl-3H-naptho[2,1-b]pyran-3-carboxylic acid;

6-chloro-7-(1,1-dimethylethyl)-2-trifluoromethyl-2H-1-benzopyran-3-carboxylic acid;

[2-(2,4-dichloro-6-ethyl-3,5-dimethyl-phenylamino)-5-propyl-phenyl]-acetic acid.

TABLE 3x
Examples of Cyclooxygenase-2 Selective Inhibitors as Embodiments

| Com- | |
|---|---|
| pound | |
| Num- | |
| ber | Structural Formula |
| B-26 | 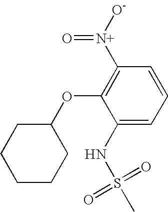 |
| | N-(2-cyclohexyloxynitrophenyl) methane sulfonamide or |
| | NS-398; |
| B-27 | 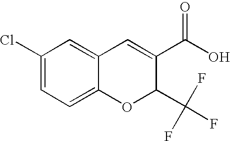 |
| | 6-chloro-2-trifluoromethyl-2H-1-benzopyran-3-carboxylic acid; |
| B-28 | 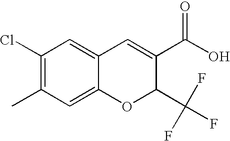 |
| | 6-chloro-7-methyl-2-trifluoromethyl-2H-1-benzopyran-3- |
| | carboxylic acid; |
| B-29 | 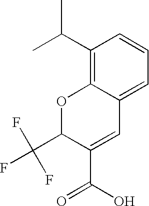 |
| | 8-(1-methylethyl)-2-trifluoromethyl-2H-1-benzopyran-3- |
| | carboxylic acid; |
| B-30 | 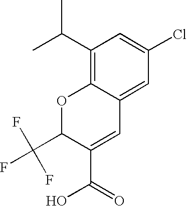 |
| | 6-chloro-8-(1-methylethyl)-2-trifluoromethyl |
| | 2-H-1-benzopyran-3-carboxylic acid; |
| B-31 | 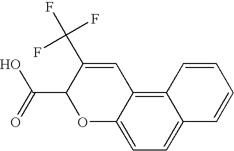 |
| | 2-trifluoromethyl-3H-naphtho[2,1-b]pyran-3- |
| | carboxylic acid; |
| B-32 | 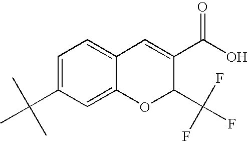 |
| | 7-(1,1-dimethylethyl)-2-trifluoromethyl-2H-1-benzopyran-3- |
| | carboxylic acid; |
| B-33 | 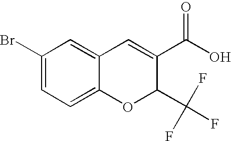 |
| | 6-bromo-2-trifluoromethyl-2H-1-benzopyran-3-carboxylic acid; |
| B-34 | 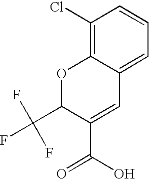 |
| | 8-chloro-2-trifluoromethyl-2H-1-benzopyran-3-carboxylic acid; |
| B-35 | 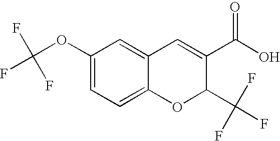 |
| | 6-trifluoromethoxy-2-trifluoromethyl-2H-1-benzopyran-3- |
| | carboxylic acid; |
| B-36 | 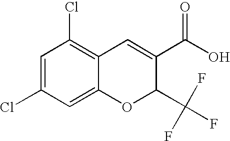 |
| | 5,7-dichloro-2-trifluoromethyl-2H-1-benzopyran-3- |
| | carboxylic acid; |
| B-37 | 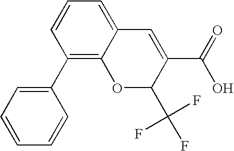 |
| | 8-phenyl-2-trifluoromethyl-2H-1-benzopyran-3- |
| | carboxylic acid; |
| B-38 | 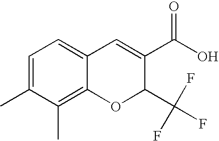 |
| | 7,8-dimethyl-2-trifluoromethyl-2H-1-benzopyran-3- |
| | carboxylic acid; |
| B-39 | 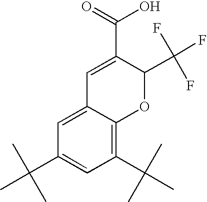 |
| | 6,8-bis(dimethylethyl)-2-trifluoro-2H-1-benzopyran-3- |
| | carboxylic acid; |
| B-40 | 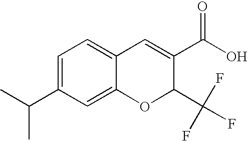 |
| | 7-(1-methylethyl)-2-trifluoromethyl-2H-1-benzopyran-3- |
| | carboxylic acid; |
| B-41 | 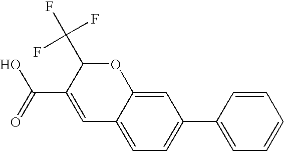 |
| | 7-phenyl-2-trifluoromethyl-2H-1-benzopyran-3-carboxylic |
| | acid; |
| B-42 | 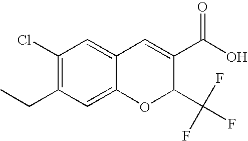 |
| | 6-chloro-7-ethyl-2-trifluoromethyl-2H-1-benzopyran-3- |
| | carboxylic acid; |
| B-43 | 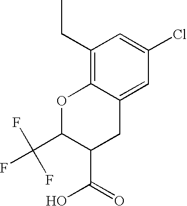 |
| | 6-chloro-8-ethyl-2-trifluoromethyl-2H-1-benzopyran-3- |
| | carboxylic acid; |
| B-44 | 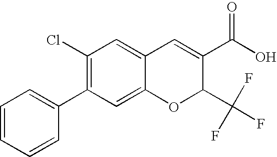 |
| | 6-chloro-7-phenyl-2-trifluoromethyl-2H-1-benzopyran-3- |
| | carboxylic acid; |
| B-45 | 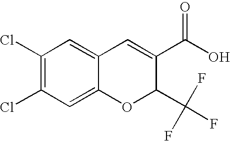 |
| | 6,7-dichloro-2-trifluoromethyl-2H-1-benzopyran-3- |
| | carboxylic acid; |
| B-46 | 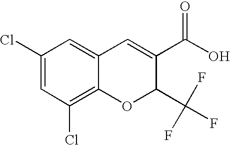 |
| | 6,8-dichloro-2-trifluoromethyl-2H-1-benzopyran-3- |
| | carboxylic acid; |
| B-47 | 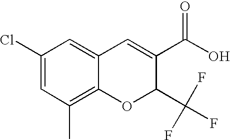 |
| | 6-chloro-8-methyl-2-trifluoromethyl-2H-1-benzopyran-3- |
| | carboxylic acid; |
| B-48 | 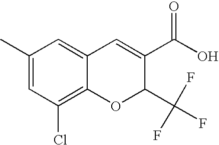 |
| | 8-chloro-6-methyl-2-trifluoromethyl-2H-1-benzopyran-3- |
| | carboxylic acid; |
| B-49 | 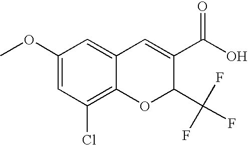 |
| | 8-chloro-6-methoxy-2-trifluoromethyl-2H-1-benzopyran-3- |
| | carboxylic acid; |
| B-50 | 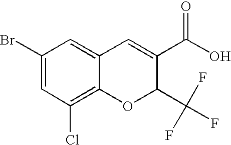 |
| | 6-bromo-8-chloro-2-trifluoromethyl-2H-1-benzopyran-3- |
| | carboxylic acid; |
| B-51 | 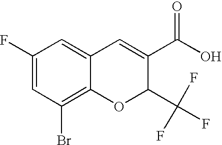 |
| | 8-bromo-6-fluoro-2-trifluoromethyl-2H-1-benzopyran-3- |
| | carboxylic acid; |
| B-52 | 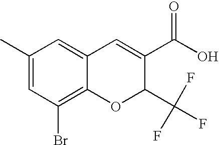 |
| | 8-bromo-6-methyl-2-trifluoromethyl-2H-1-benzopyran-3- |
| | carboxylic acid; |
| B-53 | 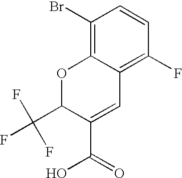 |
| | 8-bromo-5-fluoro-2-trifluoromethyl-2H-1-benzopyran-3- |
| | carboxylic acid; |
| B-54 | 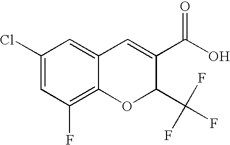 |
| | 6-chloro-8-fluoro-2-trifluoromethyl-2H-1-benzopyran-3- |
| | carboxylic acid; |
| B-55 | 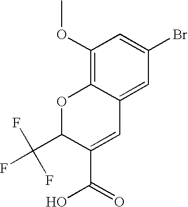 |
| | 6-bromo-8-methoxzy-2-trifluoromethyl-2H-1-benzopyran-3- |
| | carboxylic acid; |
| B-56 | 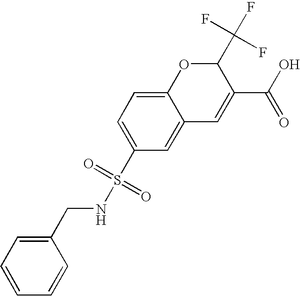 |
| | 6-[[(phenylmethyl)amino]sulfonyl]-2-trifluoromethyl- |
| | 2H-1-benzopyran-3-carboxylic acid; |
| B-57 | 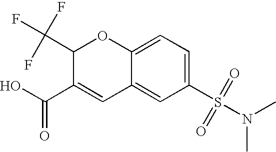 |
| | 6-[(dimethylamino)sulfonyl]-2-trifluoromethyl-2H-1- |
| | benzopyran-3-carboxylic acid; |
| B-58 | 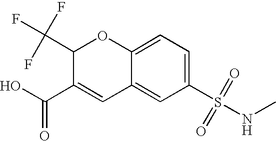 |
| | 6-[(methylamino)sulfonyl]-2-trifluoromethyl-2H-1- |
| | benzopyran-3-carboxylic acid; |
| B-59 | 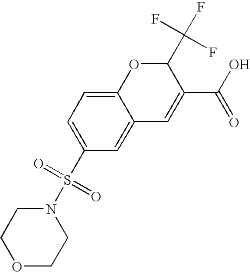 |
| | 6-[(4-morpholino)sulfonyl]-2-trifluoromethyl-2H-1- |
| | benzopyran-3-carboxylic acid; |
| B-60 | 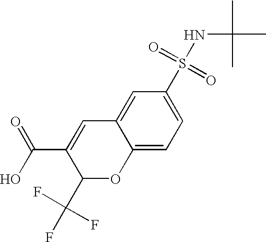 |
| | 6-[(1,1-dimethylethyl)aminosulfonyl]-2-trifluoromethyl |
| | -2H-1-benzopyran-3-carboxylic acid; |
| B-61 | 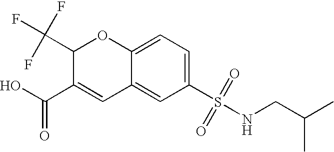 |
| | 6-[(2-methylpropyl)aminosulfonyl]-2-trifluoromethyl- |
| | 2H-1-benzopyran-3-carboxylic acid; |
| B-62 | 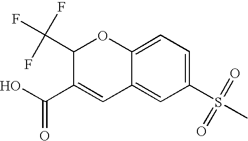 |
| | 6-methylsulfonyl-2-trifluoromethyl-2H-1-benzopyran-3- |
| | carboxylic acid; |
| B-63 | 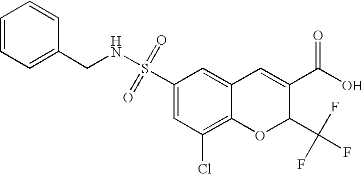 |
| | 8-chloro-6-[[(phenylmthyl)amino]sulfonyl]-2-trifluoromethyl- |
| | 2H-1-benzopyran-3-carboxylic acid; |
| B-64 | 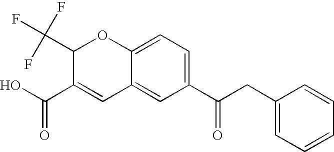 |
| | 6-phenylacetyl-2-trifluoromethyl-2H-1-benzopyran-3-carboxylic |
| | acid; |
| B-65 | 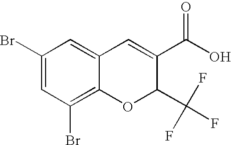 |
| | 6,8-dibromo-2-trifluoromethyl-2H-1-benzopyran-3-carboxylic |
| | acid; |
| B-66 | 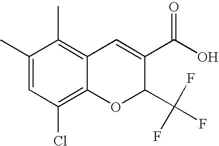 |
| | 8-chloro-5,6-dimethyl-2-trifluoromethyl-2H-1-benzopyran-3- |
| | carboxylic acid; |
| B-67 | 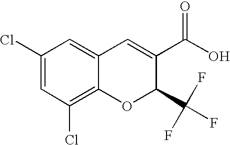 |
| | 6,8-dichloro-(S)-2-trifluoromethyl-2H-1-benzopyran-3- |
| | carboxylic acid; |
| B-68 | 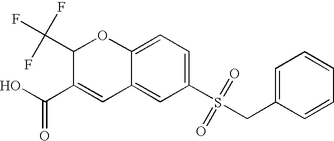 |
| | 6-benzylsulfonyl-2-trifluoromethyl-2H-1-benzopyran-3- |
| | carboxylic acid; |
| B-69 | 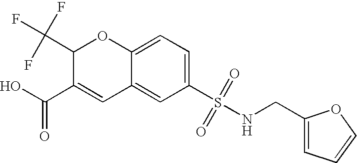 |
| | 6-[[N-(2-furylmethyl)amino]sulfonyl]-2-trifluoromethyl |
| | -2H-1-benzopyran-3-carboxylic acid; |
| B-70 | 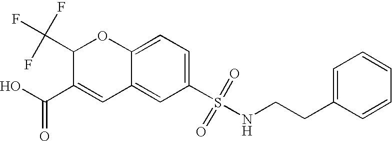 |
| | 6-[[N-(2-phenylethyl)amino]sulfonyl]-2-trifluoromethyl- |
| | 2H-1-benzopyran-3-carboxylic acid; |
| B-71 | 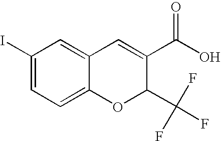 |
| | 6-iodo-2-trifluoromethyl-2H-1-benzopyran-3-carboxylic acid; |
| B-72 | 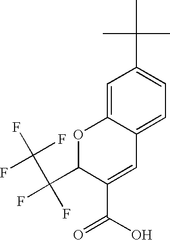 |
| | 7-(1,1-dimethylethyl)-2-pentafluoroethyl-2H |
| | -1-benzopyran-3-carboxylic acid; |
| B-73 | 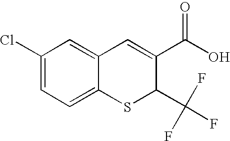 |
| | 6-chloro-2-trifluoromethyl-2H-1-benzothiopyran-3-carboxylic |
| | acid; |
| B-74 | 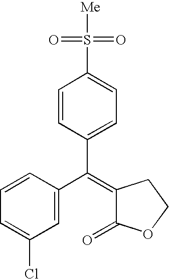 |
| | 3-[(3-chloro-phenyl)-(4-methanesulfonyl-phenyl)-methylene] |
| | -dihydro-furan-2-one or BMS-347070; |
| B-75 | 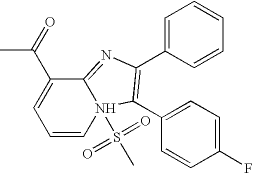 |
| | 8-acetyl-3-(4-fluorophenyl)-2-(4-methylsulfonyl)phenyl- |
| | imidazo(1,2-a)pyridine; |
| B-76 | 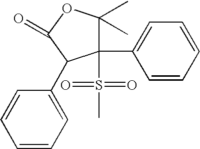 |
| | 5,5-dimethyl-4-(4-methylsulfonyl)phenyl-3-phenyl-2-(5H)- |
| | furanone; |
| B-77 | 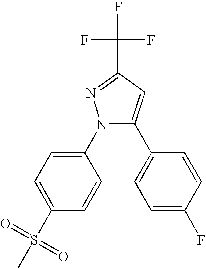 |
| | 5-(4-fluorophenyl)-1-[4-(methylsulfonyl)phenyl]-3- |
| | (trifluoromethyl)pyrazole; |
| B-78 | 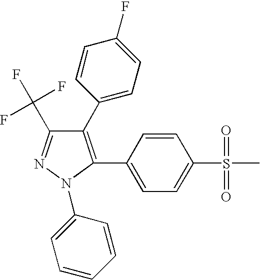 |
| | 4-(4-fluorophenyl)-5-[4-(methylsulfonyl)phenyl] |
| | -1-phenyl-3-(trifluoromethyl)pyrazole; |
| B-79 | 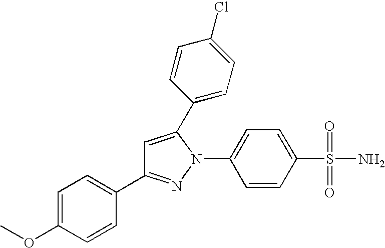 |
| | 4-(5-(4-chlorophenyl)-3-(4-methoxyphenyl)-1H-pyrazol-1-yl) |
| | benzenesulfonamide; |
| B-80 | 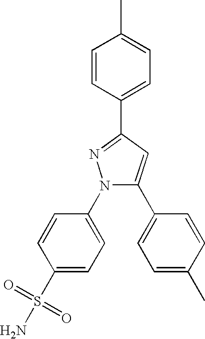 |
| | 4-(3,5-bis(4-methylphenyl)-1H-pyrazol-1-yl) |
| | benzenesulfonamide; |
| B-81 | 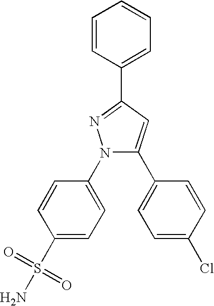 |
| | 4-(5-(4-chlorophenyl)-3-phenyl-1H-pyrazol-1-yl) |
| | benzenesulfonamide; |
| B-82 | 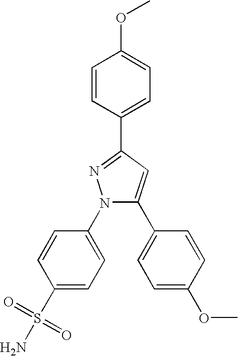 |
| | 4-(3,5-bis(4-methoxyphenyl)-1H-pyrazol-1-yl) |
| | benzenesulfonamide; |
| B-83 | 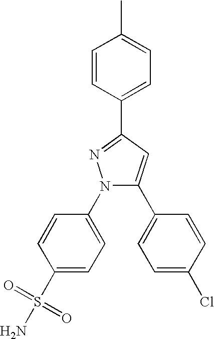 |
| | 4-(5-(4-chlorophenyl)-3-(4-methylphenyl)-1H-pyrazol-1- |
| | yl)benzenesulfonamide; |
| B-84 | 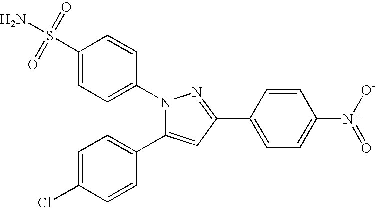 |
| | 4-(5-(4-chlorophenyl)-3-(4-nitrophenyl)-1H-pyrazol-1- |
| | yl)benzenesulfonamide; |
| B-85 | 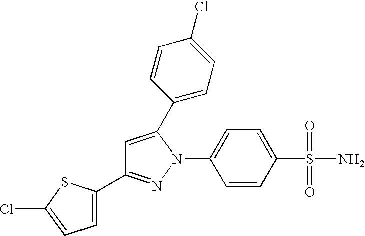 |
| | 4-(5-(4-chlorophenyl)-3-(5-chloro-2-thienyl)-1H-pyrazol-1- |
| | yl)benzenesulfonamide; |
| B-86 | 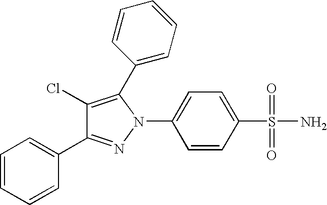 |
| | 4-(4-chloro-3,5-diphenyl-1H-pyrazol-1-yl)benzenesulfonamide; |
| B-87 | 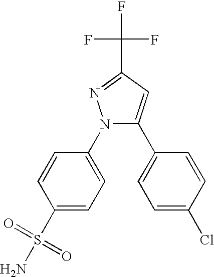 |
| | 4-[5-(4-chlorophenyl)-3-(trifluoromethyl)-1H-pyrazol-1- |
| | yl]benzenesulfonamide; |
| B-88 | 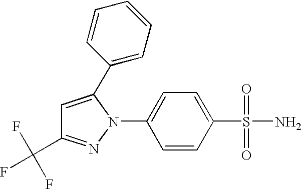 |
| | 4-[5-phenyl-3-(trifluoromdethyl)-1H-pyrazol-1- |
| | yl]benzenesulfonamide; |
| B-89 | 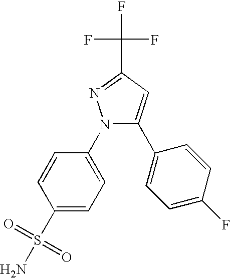 |
| | 4-[5-(4-fluorophenyl)-3-(trifluoromethyl)-1H-pyrazol-1- |
| | yl]benzenesulfonamide; |
| B-90 | 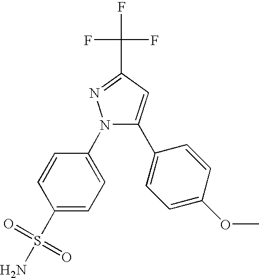 |
| | 4[5-(4-methoxyphenyl)-3-(trifluoromethyl)-1H-pyrazol-1- |
| | yl]benzenesulfonamide; |
| B-91 | 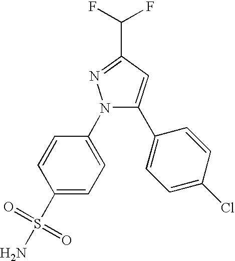 |
| | 4-[5-(4-chlorophenyl)-3-(difluoromethyl)-1H-pyrazol-1- |
| | yl]benzenesulfonamide; |
| B-92 | 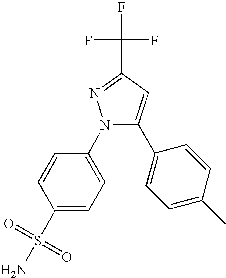 |
| | 4-[5-(4-methylphenyl)-3-(trifluoromethyl)-1H-pyrazol-1- |
| | yl]benzenesulfonamide; |
| B-93 | 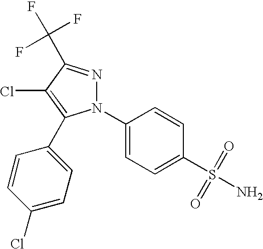 |
| | 4-[4-chloro-5-(4-chlorophenyl)-3-(trifluoromethyl)-1H- |
| | pyrazol-1-yl] |
| | benzenesulfonamide; |
| B-94 | 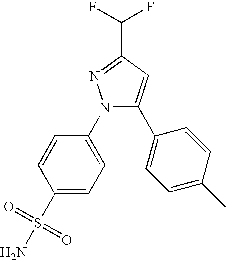 |
| | 4-[3-(difluoromethyl)-5-(4-methylphenyl)-1H-pyrazol-1- |
| | yl]benzenesulfonamide; |
| B-95 | 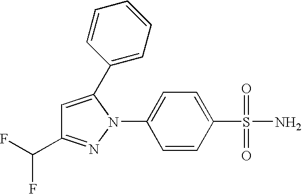 |
| | 4-[3-(difluoromethyl)-5-phenyl-1H-pyrazol-1- |
| | yl]benzenesulfonamide; |
| B-96 | 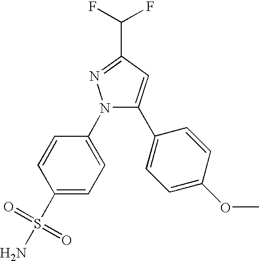 |
| | 4-[3-(difluoromethyl)-5-(4-methoxyphenyl)-1H-pyrazol-1- |
| | yl]benzenesulfonamide; |
| B-97 | 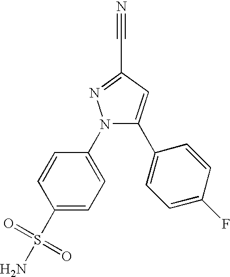 |
| | 4-[3-cyano-5-(4-fluorophenyl)-1H-pyrazol-1- |
| | yl]benzenesulfonamide; |
| B-98 | 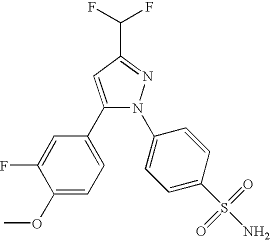 |
| | 4-[3-(difluoromethyl)-5-(3-fluoro-4-methoxyphenyl)-1H- |
| | pyrazol-1-yl]benzenesulfonamide; |
| B-99 | 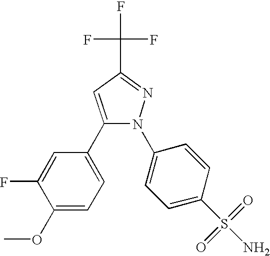 |
| | 4-[5-(3-fluoro-4-methoxyphenyl)-3-(trifluoromethyl)-1H- |
| | pyrazol-1-yl] |
| | benzenesulfonamide; |
| B-100 | 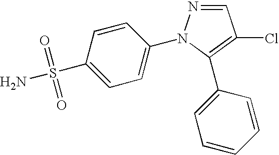 |
| | 4-[4-chloro-5-phenyl-1H-pyrazol-1-yl]benzenesulfonamide; |
| B-101 | 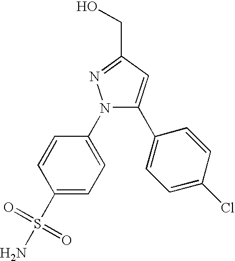 |
| | 4-[5-(4-chlorophenyl)-3-(hydroxymethyl)-1H-pyrazol-1- |
| | yl]benzenesulfonamide; |
| B-102 | 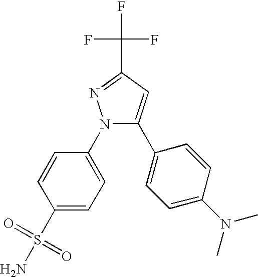 |
| | 4-[5-(4-(N,N-dimethylamino)phenyl)-3-(trifluoromethyl) |
| | -1H-pyrazol-1-yl]benzenesulfonamide; |
| B-103 | 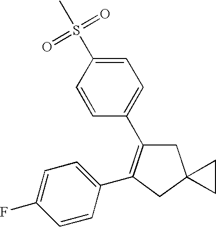 |
| | 5-(4-fluorophenyl)-6-[4-(methylsulfonyl)phenyl]spiro |
| | [2.4]hept-5-ene; |
| B-104 | 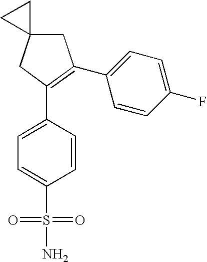 |
| | 4-[6-(4-fluorophenyl)spiro[2.4]hept-5-en-5- |
| | yl]benzenesulfonamide; |
| B-105 | 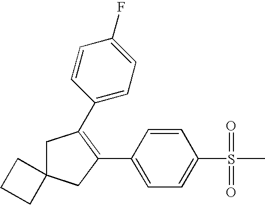 |
| | 6-(4-fluorophenyl)-7-[4-methylsulfonyl)phenyl]spiro |
| | [3.4]oct-6-ene; |
| B-106 | 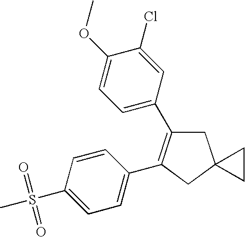 |
| | 5-(3-chloro-4-methoxyphenyl)-6-[4-(methylsulfonyl)phenyl]spiro |
| | [2,4]hept-5-ene; |
| B-107 | 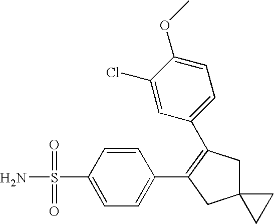 |
| | 4-[6-(3-chloro-4-methoxyphenyl)spiro[2,4]hept- |
| | 5-en-5-yl]benzenesulfonamide; |
| B-108 | 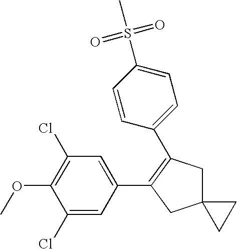 |
| | 5-(3,5-dichloro-4-methoxyphenyl)-6-[4-(methylsulfonyl)phenyl] |
| | spiro[2.4]hept-5-ene; |
| B-109 | 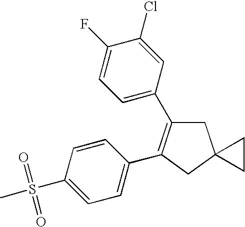 |
| | 5-(3-chloro-4-fluorophenyl)-6-[4-(methylsulfonyl)phenyl]spiro |
| | [2,4]hept-5-ene; |
| B-110 | 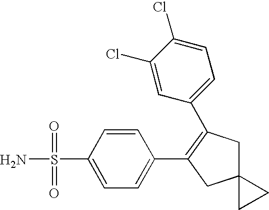 |
| | 4-[6-(3,4-dichlorophenyl)spiro[2,4]hept-5-en-5- |
| | yl]benzenesulfonamide; |
| B-111 | 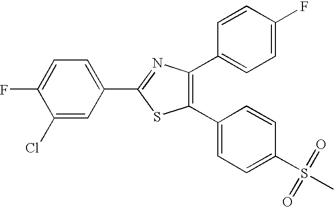 |
| | 2-(3-chloro-4-fluorophenyl)-4-(4-fluorophenyl)-5-(4- |
| | methylsulfonylphenyl)thiazole; |
| B-112 | 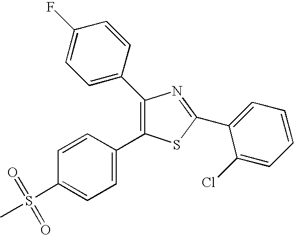 |
| | 2-(2-chlorophenyl)-4-(4-fluorophenyl)-5-(4- |
| | methylsulfonylphenyl)thiazole; |
| B-113 | 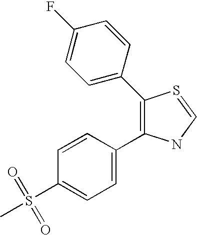 |
| | 5-(4-fluorophenyl)-4-(4-methylsulfonylphenyl)-2-methylthiazole; |
| B-114 | 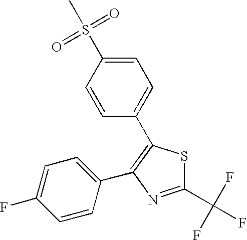 |
| | 4-(4-fluorophenyl)-5-(4-methylsulfonylphenyl)-2- |
| | trifluoromethylthiazole; |
| B-115 | 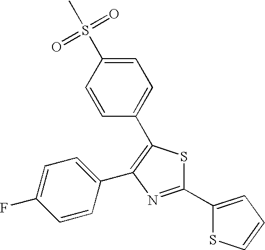 |
| | 4-(4-fluorophenyl)-5-(4-methylsulfonylphenyl)-2-(2- |
| | thienyl)thiazole; |
| B-116 | 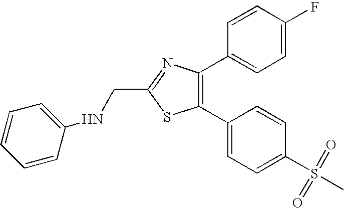 |
| | 4-(4-fluorophenyl)-5-(4-methylsulfonylphenyl)-2- |
| | benzylaminothiazole; |
| B-117 | 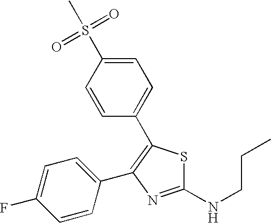 |
| | 4-(4-fluorophenyl)-5-(4-methylsulfonylphenyl)-2- |
| | (1-propylamino)thiazole; |
| B-118 | 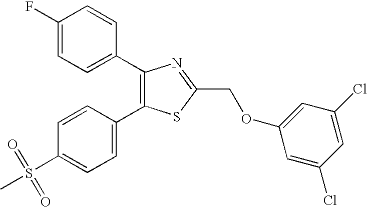 |
| | 2-((3,5-dichlorophenoxy)methyl)-4-(4-fluorophenyl)- |
| | 5-[4-(methylsulfonyl)phenyl]thiazole; |
| B-119 | 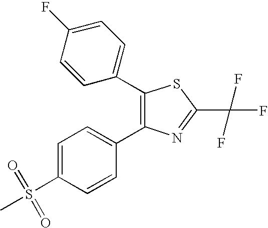 |
| | 5-(4-fluorophenyl)-4-(4-methylsulfonylphenyl)-2- |
| | trifluoromethylthiazole; |
| B-120 | 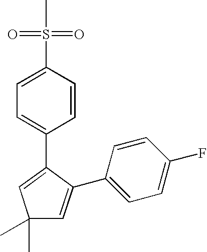 |
| | 1-methylsulfonyl-4-[1,1-dimethyl-4-(4-fluorophenyl) |
| | cyclopenta-2,4-dien-3-yl]benzene; |
| B-121 | 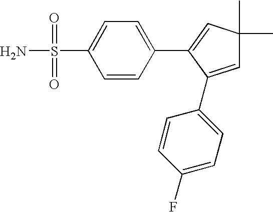 |
| | 4-[4-(4-fluorophenyl)-1,1-dimethylcyclopenta-2,4-dien-3-yl] |
| | benzenesulfonamide; |
| B-122 | 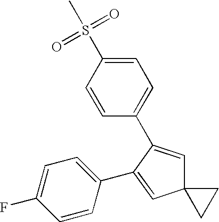 |
| | 5-(4-fluorophenyl)-6-[4-(methylsulfonyl)phenyl]spiro |
| | [2,4]hepta-4,6-diene; |
| B-123 | 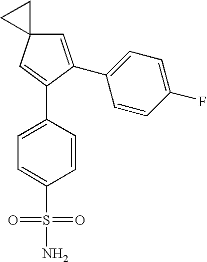 |
| | 4-[6-(4-fluorophenyl)spiro[2.4]hepta-4,6-dien-5- |
| | yl]benzenesulfonamide; |
| B-124 | 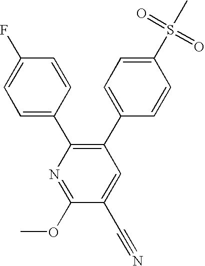 |
| | 6-(4-fluorophenyl)-2-methoxy-5-[4-(methylsulfonyl)phenyl] |
| | -pyridine-3-carbonitrile; |
| B-125 | 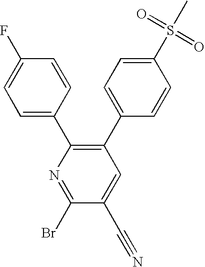 |
| | 2-bromo-6-(4-fluorophenyl)-5-[4-(methylsulfonyl)phenyl] |
| | -pyridine-3-carbonitrile; |
| B-126 | 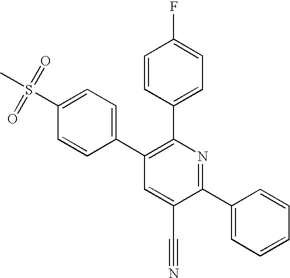 |
| | 6-(4-fluorophenyl)-5-[4-(methylsulfonyl)phenyl]- |
| | 2-phenyl-pyridine-3-carbonitrile; |
| B-127 | 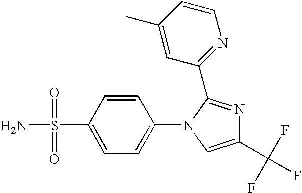 |
| | 4-[2-(4-methylpyridin-2-yl)-4-(trifluoromethyl)-1H- |
| | imidazol-1-yl]benzenesulfonamide; |
| B-128 | 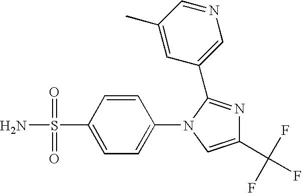 |
| | 4-[2-(5-methylpyridin-3-yl)-4-(trifluoromethyl)-1H- |
| | imidazol-1-yl]benzenesulfonamide; |
| B-129 | 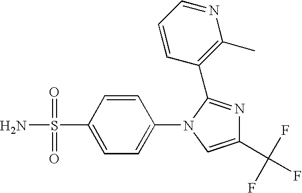 |
| | 4-[2-(2-methylpyridin-3-yl)-4-(trifluoromethyl)-1H- |
| | imidazol-1-yl]benzenesulfonamide; |
| B-130 | 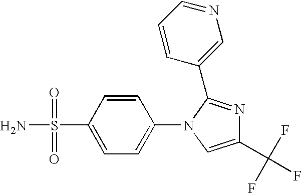 |
| | 3-[1-[4-(methylsulfonyl)phenyl]-4-(trifluoromethyl)-1H- |
| | imidazol-2-yl]pyridine; |
| B-131 | 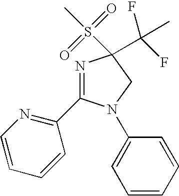 |
| | 2-[1-[4-(methylsulfonyl)phenyl-4-(trifluoromethyl)-1H- |
| | imidazol-2-yl]pyridine; |
| B-132 | 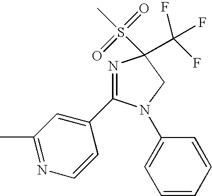 |
| | 2-methyl-4-[1-[4-(methylsulfonyl)phenyl-4-(trifluoromethyl)] |
| | -1H-imidazol-2-yl]pyridine; |
| B-133 | 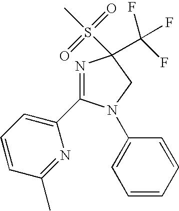 |
| | 2-methyl-6-[1-[4-(methylsulfonyl)phenyl-4-(trifluoromethyl)] |
| | -1H-imidazol-2-yl]pyridine; |
| B-134 | 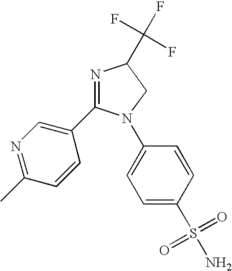 |
| | 4-[2-(6-methylpyridin-3-yl)-4-(trifluoromethyl)-1H-imidazol-1- |
| | yl]benzenesulfonamide; |
| B-135 | 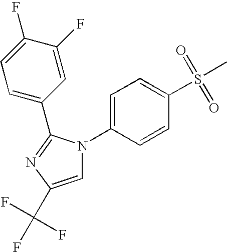 |
| | 2-(3,4-difluorophenyl)-1-[4-(methylsulfonyl)phenyl] |
| | -4-(trifluoromethyl)-1H-imidazole; |
| B-136 | 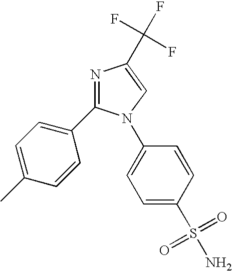 |
| | 4-[2-(4-methylphenyl)-4-(trifluoromethyl)-1H-imidazol-1- |
| | yl]benzenesulfonamide; |
| B-137 | 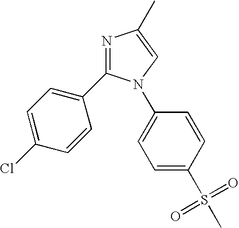 |
| | 2-(4-chlorophenyl)-1-[4-(methylsulfonyl)phenyl]-4-methyl- |
| | 1H-imidazole; |
| B-138 | 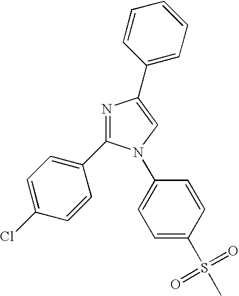 |
| | 2-(4-chlorophenyl)-1-[4-(methylsulfonyl)phenyl]-4-phenyl- |
| | 1H-imidazole; |
| B-139 | 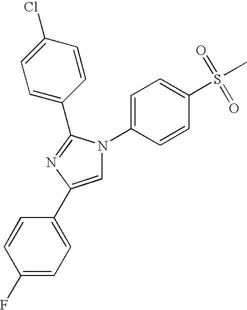 |
| | 2-(4-chlorophenyl)-4-(4-fluorophenyl)-1-[4- |
| | methylsulfonyl)phenyl]-1H-imidazole; |
| B-140 | 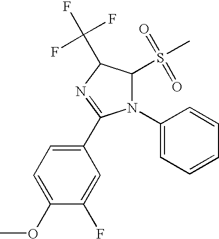 |
| | 2-(3-fluoro-4-methoxyphenyl)-1-[4-(methylsulfonyl)phenyl |
| | -4-(trifluoromethyl)]-1H-imidazole; |
| B-141 | 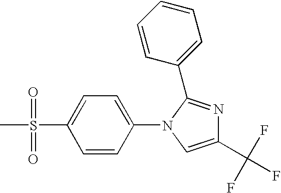 |
| | 1-[4-(methylsulfonyl)phenyl]-2-phenyl-4-trifluoromethyl- |
| | 1H-imidazole; |
| B-142 | 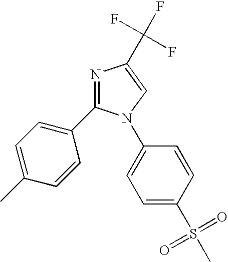 |
| | 2-(4-methylphenyl)-1-[4-(methylsulfonyl)phenyl]-4- |
| | trifluoromethyl-1H-imidazole; |
| B-143 | 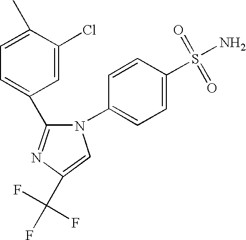 |
| | 4-[2-(3-chloro-4-methylphenyl)-4-(trifluoromethyl) |
| | -1H-imidazol-1-yl]benzenesulfonamide; |
| B-144 | 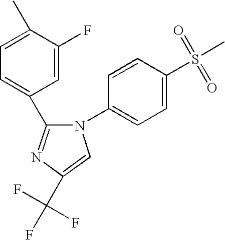 |
| | 2-(3-fluoro-5-methylphenyl)-1-[4-(methylsulfonyl)phenyl] |
| | -4-(trifluoromethyl)-1H-imidazole; |
| B-145 | 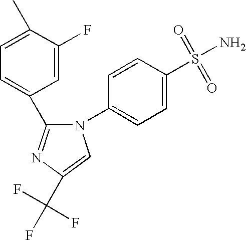 |
| | 4-[2-(3-fluoro-5-methylphenyl)-4-(trifluoromethyl |
| | -1H-imidazole-1-yl]benzenesulfonamide; |
| B-146 | 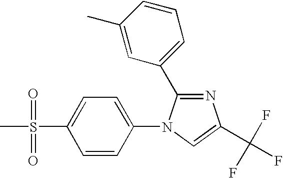 |
| | 2-(3-methylphenyl)-1-[4-(methylsulfonyl)phenyl]-4- |
| | trifluoromethyl-1H-imidazole; |
| B-147 | 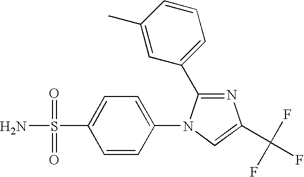 |
| | 4-[2-(3-methylphenyl)-4-trifluoromethyl-1H-imidazol-1- |
| | yl]benzenesulfonamide; |
| B-148 | 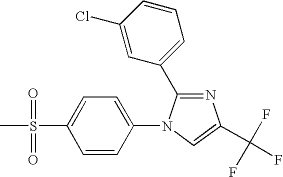 |
| | 1-[4-(methylsulfonyl)phenyl]-2-(3-chlorophenyl)-4- |
| | trifluoromethyl-1H-imidazole |
| B-149 | 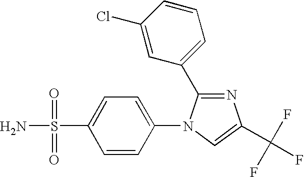 |
| | 4-[2-(3-chlorophenyl)-4-trifluoromethyl-1H-imidazol-1- |
| | yl]benzenesulfonamide; |
| B-150 | 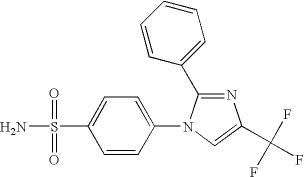 |
| | 4-[2-phenyl-4-trifluoromethyl-1H-imidazol-1- |
| | yl]benzenesulfonamide; |
| B-151 | 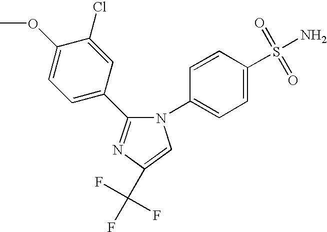 |
| | 4-[2-(4-methoxy-3-chlorophenyl)-4-trifluoromethyl-1H- |
| | imidazol-1-yl]benzenesulfonamide; |
| B-152 | 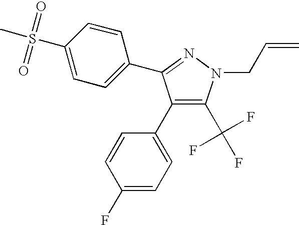 |
| | 1-allyl-4-(4-fluorophenyl)-3-[4-(methylsulfonyl)phenyl] |
| | -5-(trifluoromethyl)-1H-pyrazole; |
| B-153 | 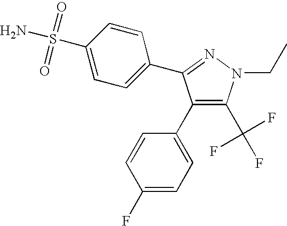 |
| | 4-[1-ethyl-4-(4-fluorophenyl)-5-(trifluoromethyl)-1H-pyrazol- |
| | 3-yl]benzenesulfonamide; |
| B-154 | 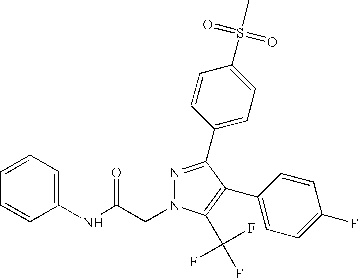 |
| | N-phenyl-[4-(4-fluorophenyl)-3-[4-(methylsulfonyl)phenyl] |
| | -5-(trifluoromethyl)-1H-pyrazol-1-yl]acetamide; |
| B-155 | 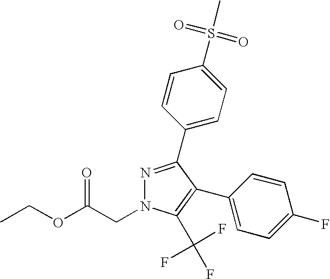 |
| | ethyl[4-(4-fluorophenyl)-3-[4-(methylsulfonyl)phenyl] |
| | -5-(trifluoromethyl)-1H-pyrazol-1-yl]acetate; |
| B-156 | 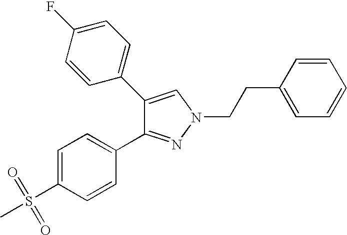 |
| | 4-(4-fluorophenyl)-3-[4-(methylsulfonyl)phenyl]-1-(2- |
| | phenylethyl)-1H-pyrazole; |
| B-157 | 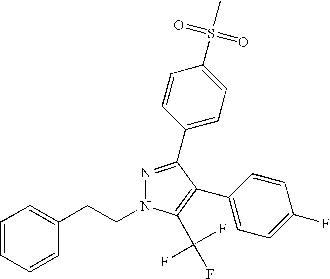 |
| | 4-(4-fluorophenyl)-3-[4-(methylsulfonyl)phenyl] |
| | -1-(2-phenylethyl)-5-(trifluoromethyl)pyrazole; |
| B-158 | 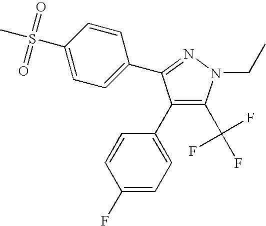 |
| | 1-ethyl-04-(4-fluorophenyl)-3-[4-methylsulfonyl)phenyl] |
| | -5-(trifluoromethyl)-1H-pyrazole; |
| B-159 | 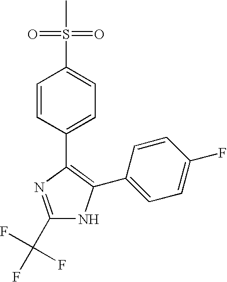 |
| | 5-(4-fluorophenyl)-4-(4-methylsulfonylphenyl) |
| | -2-trifluoromethyl-1H-imidazole; |
| B-160 | 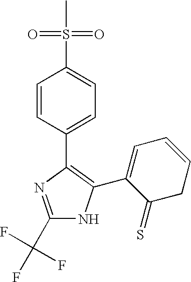 |
| | 4-[4-(methylsulfonyl)phenyl]-4-(2-thiophenyl)-2- |
| | (trifluoromethyl)-1H-imidazole; |
| B-161 | 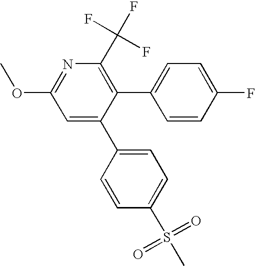 |
| | 5-(4-fluorophenyl)-2-methoxy-4-[4-(methylsulfonyl)phenyl]- |
| | 6-(trifluoromethyl)pyridine; |
| B-162 | 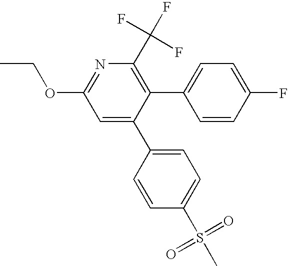 |
| | 2-ethoxy-5-(4-fluorophenyl)-4-[4-(methylsulfonyl)phenyl] |
| | -6-(trifluoromethyl)pyridine; |
| B-163 | 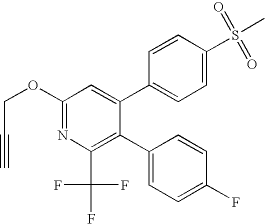 |
| | 5-(4-fluorophenyl)-4-[4-(methylsulfonyl)phenyl] |
| | -2-(2-propynyloxy)-6-(trifluoromethyl)pyridine; |
| B-164 | 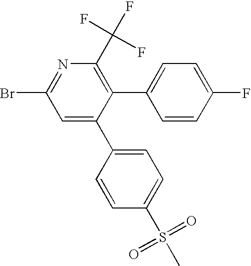 |
| | 2-bromo-5-(4-fluorophenyl)-4-[4-(methylsulfonyl)phenyl] |
| | -6-(trifluoromethyl)pyridine; |
| B-165 | 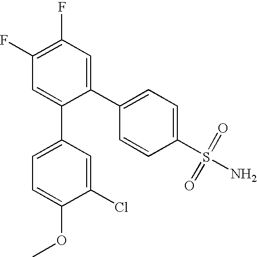 |
| | 4-[2-(3-chloro-4-methoxyphenyl)-4,5- |
| | difluorophenyl]benzenesulfonamide; |
| B-166 | 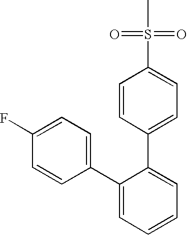 |
| | 1-(4-fluorophenyl)-2-[4-methylsulfonyl)phenyl]benzene; |
| B-167 | 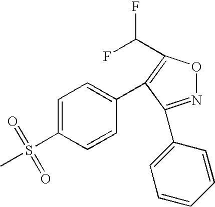 |
| | 5-difluoromethyl-4-(4-methylsulfonylphenyl)-3-phenylisoxazole; |
| B-168 | 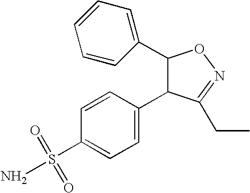 |
| | 4-[3-ethyl-5-phenylisoxazol-4-yl]benzenesulfonamide; |
| B-169 | 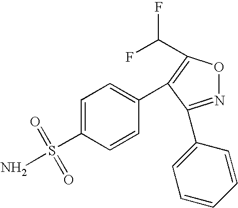 |
| | 4-[5-difluoromethyl-3-phenylisoxazol-4-yl]benzenesulfonamide; |
| B-170 | 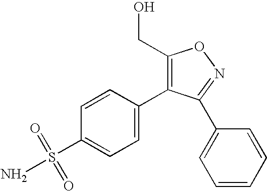 |
| | 4-[5-hydroxymethyl-3-phenylisoxazol-4-yl]benzenesulfonamide; |
| B-171 | 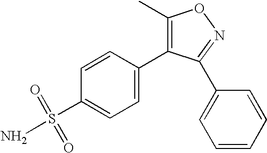 |
| | 4-[5-methyl-3-phenyl-isoxazol-4-yl]benzenesulfonamide; |
| B-172 | 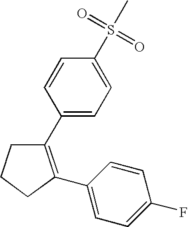 |
| | 1-[2-(4-fluorophenyl)cyclopenten-1-yl]-4- |
| | (methylsulfonyl)benzene; |
| B-173 | 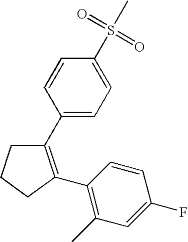 |
| | 1-[2-(4-fluoro-2-methylphenyl)cyclopenten-1-yl]-4- |
| | (methylsulfonyl)benzene; |
| B-174 | 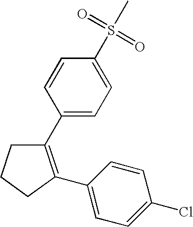 |
| | 1-[2-(4-chlorophenyl)cyclopenten-1-yl]-4- |
| | (methylsulfonyl)benzene; |
| B-175 | 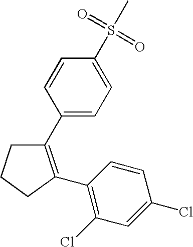 |
| | 1-[2-(2,4-dichlorophenyl)cyclopenten-1-yl]-4- |
| | (methylsulfonyl)benzene; |
| B-176 | 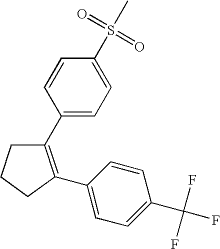 |
| | 1-[2-(4-trifluoromethylphenyl)cyclopetne-1-yl]-4- |
| | (methylsulfonyl)benzene; |
| B-177 | 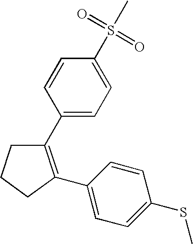 |
| | 1-[2-(4-methylthiophenyl)cyclopenten-1-yl]-4- |
| | (methylsulfonyl)benzene; |
| B-178 | 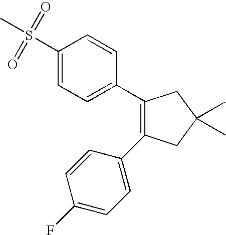 |
| | 1-[2-(4-fluorophenyl)-4,4-dimethylcyclopenten-1-yl]- |
| | 4-(methylsulfonyl)benzene; |
| B-179 | 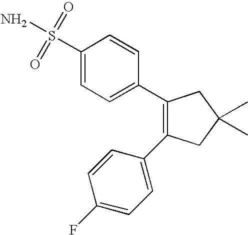 |
| | 4-[2-(4-fluorophenyl)-4,4-dimethylcyclopenten-1- |
| | yl]benzenesulfonamide; |
| B-180 | 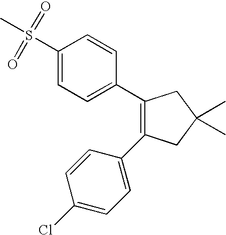 |
| | 1-[2-(3-chlorophenyl)-4,4-dimethylcyclopenten-1- |
| | yl]-4-(methylsulfonyl)benzene; |
| B-181 | 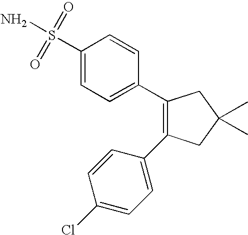 |
| | 4-[2-(4-chlorophenyl)-4,4-dimethylcyclopenten-1- |
| | yl]benzenesulfonamide; |
| B-182 | 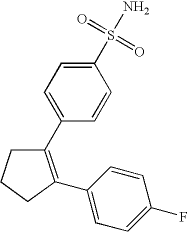 |
| | 4-[2-(4-fluorophenyl)cyclopenten-1-yl]benzenesulfonamide; |
| B-183 | 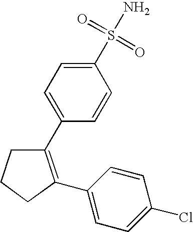 |
| | 4-[2-(4-chlorophenyl)cyclopenten-1-yl]benzenesulfonamide; |
| B-184 | 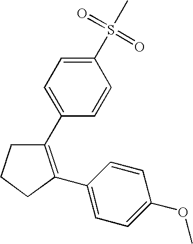 |
| | 1-[2-(4-methoxyphenyl)cyclopenten-1-yl]-4- |
| | (methylsulfonyl)benzene; |
| B-185 | 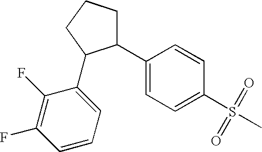 |
| | 1-[2-(2,3-difluorophenyl)cyclopeten-1-yl]-4- |
| | (methylsulfonyl)benzene; |
| B-186 | 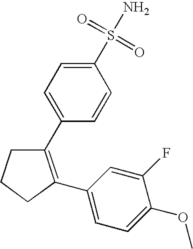 |
| | 4-[2-(3-fluoro-4-methoxyphenyl)cyclopenten-1- |
| | yl]benzenesulfonamide; |
| B-187 | 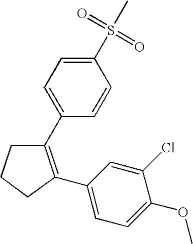 |
| | 1-[2-(3-chloro-4-methoxyphenyl)cyclopenten-1-yl]-4- |
| | (methylsulfonyl)benzene; |
| B-188 | 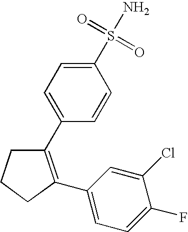 |
| | 4-[2-(3-chloro-4-fluorophenyl)cyclopenten-1- |
| | yl]benzenesulfonamide; |
| B-189 | 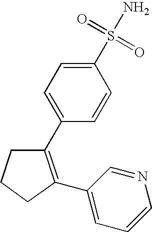 |
| | 4-[2-(2-methylpyridin-5-yl)cyclopenten-1- |
| | yl]benzenesulfonamide; |
| B-190 | 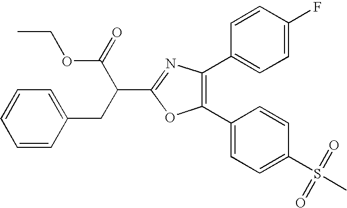 |
| | ethyl 2-[4-(4-fluorophenyl)-5-[4- |
| | (methylsulfonyl)phenyl]oxazol-2-yl]-2-benzyl-acetate; |
| B-191 | 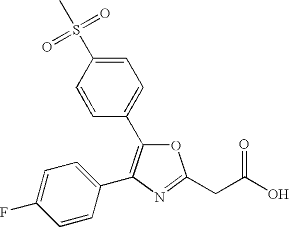 |
| | 2-[4-(4-fluorophenyl)-5-[4- |
| | (methylsulfonyl)phenyl]oxazol-2-yl]acetic acid; |
| B-192 | 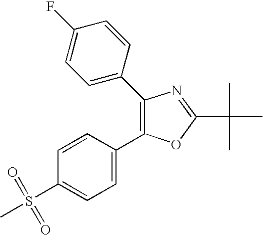 |
| | 2-(tert-butyl)-4-(4-fluorophenyl)-5-[4- |
| | (methylsulfonyl)phenyl]oxazole; |
| B-193 | 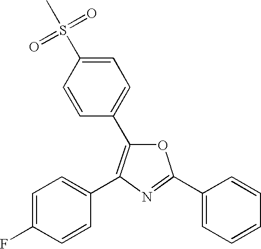 |
| | 4-(4-fluorophenyl)-5-[4-(methylsulfonyl)phenyl]- |
| | 2-phenyloxazole; |
| B-194 | 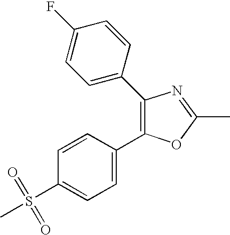 |
| | 4-(4-fluorophenyl)-2-methyl-5-[4- |
| | (methylsulfonyl)phenyl]oxazole; |
| B-195 | 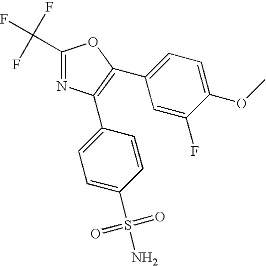 |
| | 4-[5-(3-fluoro-4-methoxyphenyl)-2- |
| | trifluoromethyl-4-oxazolyl]benzenesulfonamide; |
| B-196 | 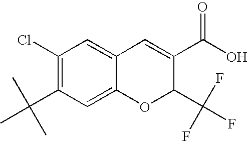 |
| | 6-chloro-7-(1,1-dimethylethyl)-2-trifluoromethyl-2H- |
| | -1-benzopyran-3-carboxylic acid; |
| B-197 | 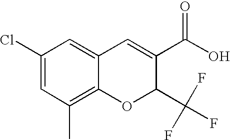 |
| | 6-chloro-8-methyl-2-trifluoromethyl-2H-1-benzopyran-3- |
| | carboxylic acid; |
| B-198 | 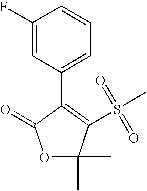 |
| | 5,5-dimethyl-3-(3-fluorophenyl)-4-methylsulfonyl-2(5H)- |
| | furanone; |
| B-199 | 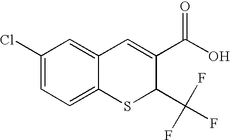 |
| | 6-chloro-2-trifluoromethyl-2H-1-benzthiopyran-3- |
| | carboxylic acid; |
| B-200 | 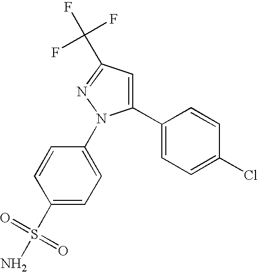 |
| | 4-[5-(4-chlorophenyl)-3-(trifluoromethyl)-1H-pyrazol- |
| | 1-yl]benzenesulfonamide; |
| B-201 | 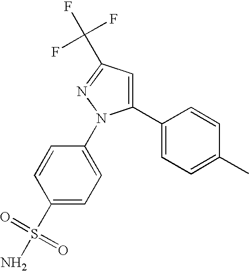 |
| | 4-[5-(4-methylphenyl)-3-(trifluoromethyl)-1H-pyrazol- |
| | 1-yl]benzenesulfonamide; |
| B-202 | 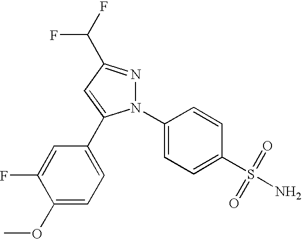 |
| | 4-[5-(4-fluoro-4-methoxyphenyl)-3-(difluoromethyl) |
| | 1-H-pyrazol-1-yl]benzenesulfonamide; |
| B-203 | 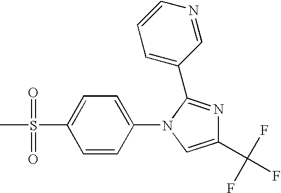 |
| | 3-[1-[4-(methylsulfonyl)phenyl]-4-trifluoromethyl- |
| | 1H-imidazol-2-yl]pyridine; |
| B-204 | 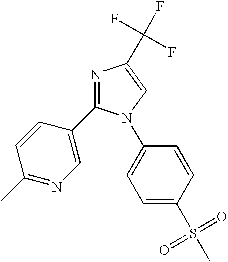 |
| | 2-methyl-5-[1-[4-(methylsulfonyl)phenyl]-4-trifluoromethyl |
| | -1H-imidazol-2-yl]pyridine; |
| B-205 | 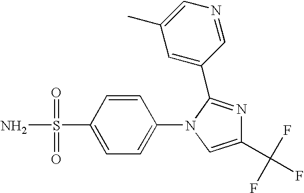 |
| | 4-[2-(5-methylpyridin-3-yl)-4-(trifluoromethyl) |
| | -1H-imidazol-1-yl]benzenesulfonamide; |
| B-206 | 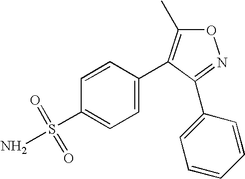 |
| | 4-[5-methyl-3-phenylisoxazol-4-yl]benzenesulfonamide; |
| B-207 | 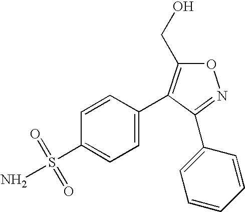 |
| | 4-[5-hydroxymethyl-3-phenylisoxazol-4-yl]benzenesulfonamide; |
| B-208 | 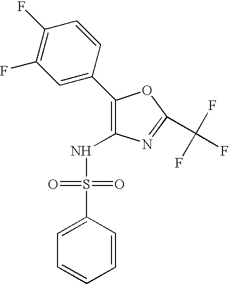 |
| | [2-trifluoromethyl-5-(3,4-difluorophenyl)-4- |
| | oxazolyl]benzenesulfonamide; |
| B-209 | 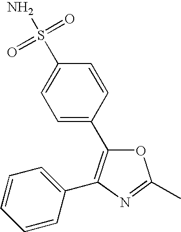 |
| | 4-[2-methyl-4-phenyl-5-oxazolyl]benzenesulfonamide; |
| B-210 | 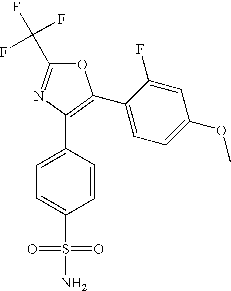 |
| | 4-[5-(2-fluoro-4-methoxyphenyl)-2-trifluoromethyl-4- |
| | oxazolyl]benzenesulfonamide; |
| B-211 | 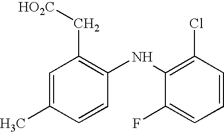 |
| B-212 | 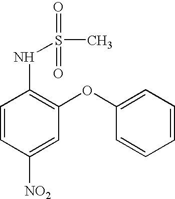 |
| | N-(4-nitro-2-phenoxy-phenyl)-methanesulfonamide or |
| | Nimesulide |
| B-213 | 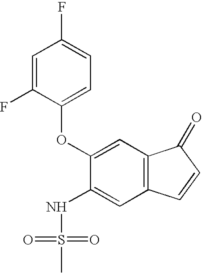 |
| | N-[6-(2,4-difluoro-phenoxy)-1-oxo-inden-5-yl]- |
| | methanesulfonamide or Flosulide |
| B-214 | 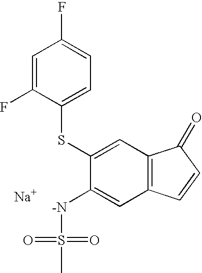 |
| | N-[6-(2,4-difluoro-phenylsulfonanyl)-1-oxo-1H-inden-5- |
| | yl]-methanesulfonamide, |
| | sodium salt, or L-745337 |
| B-215 | 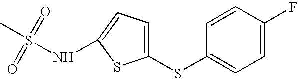 |
| | N-[5-(4-fluoro-phenylsulfanyl)-thiophen-2-yl]- |
| | methanesulfonamide or RWJ-63556 |
| B-216 | 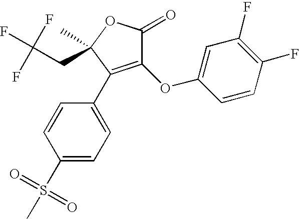 |
| | 3-(3,4-difluoro-phenoxy)-4-(4-methanesulfonyl-phenyl)-5- |
| | methyl-5-(2,2,2-trifluoro- |
| | ethyl)-5H-furan-2-one or L-784512 |
| B-217 | 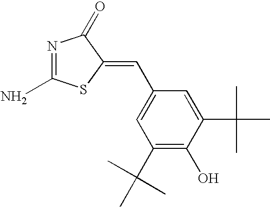 |
| | (5Z)-2-amino-5-[[3,5-bis(1,1-dimethylethyl)- |
| | 4-hydroxyphenyl]methylene] |
| | -4(5H)-thiazolone or Darbufelone |
| B-218 | CS-502 |
| B-219 | LAS-34475 |
| B-220 | LAS-34555 |
| B-221 | S-33516 |
| B-222 | SD-8381 |
| B-223 | L-783003 |
| B-224 | 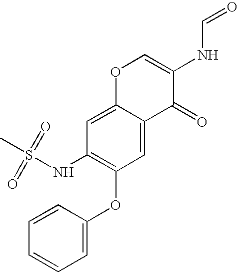 |
| | N-[3-(formylamino)-4-oxo-6-phenoxy-4H-1-benzopyran-7-yl] |
| | -methanesulfonamide or T1614 |
| B-225 | D-1367 |
| B-226 | L-748731 |
| B-227 | 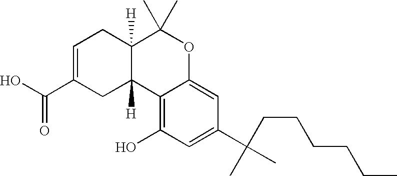 |
| | (6aR,10aR)-3-(1,1-dimethylheptyl)-6a,7,10,10a-tetrahydro- |
| | 1-hydroxy-6,6-dimethyl-6H-dibenzo[b,d]pyran-9- |
| | carboxylic acid or CT3 |
| B-228 | CGP-28238 |
| B-229 | 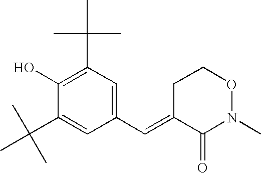 |
| | 4-[[3,5-bis(1,1-dimethylethyl)-4-hydroxyphenyl]methylene] |
| | dihydro-2-methyl-2H-1,2-oxazin-3(4H)-one or BF-389 |
| B-230 | GR-253035 |
| B-231 | 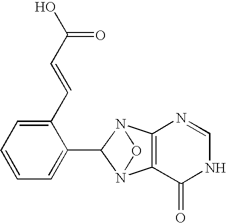 |
| | 2-(6-dioxo-9H-pyurin-8-yl)cinnamic acid |
| B-232 | S-2474 |
| B-233 | 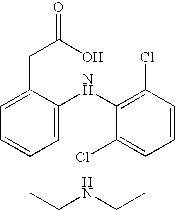 |
| B-234 | 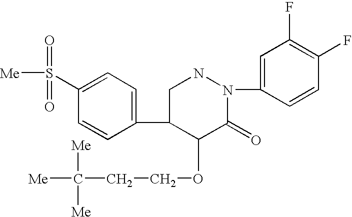 |
| B-235 | 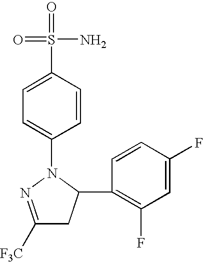 |
| B-236 | 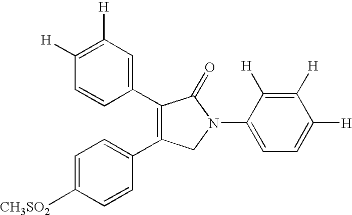 |
| B-237 | 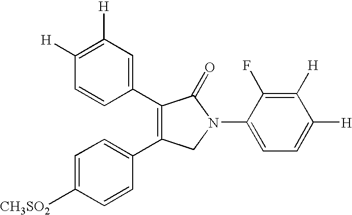 |
| B-238 | 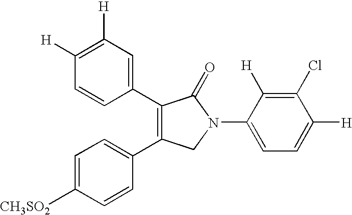 |
| B-239 | 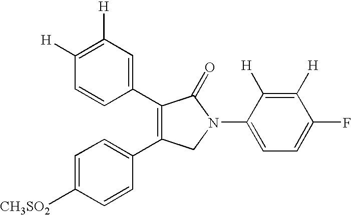 |
| B-240 | 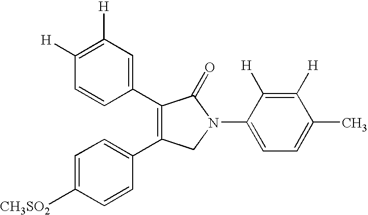 |
| B-241 | 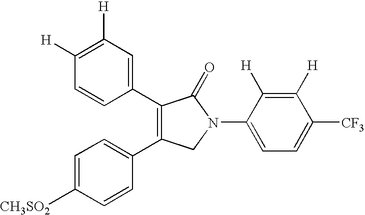 |
| B-242 | 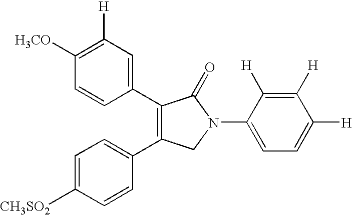 |
| B-243 | 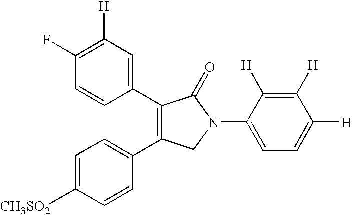 |
| B-244 | 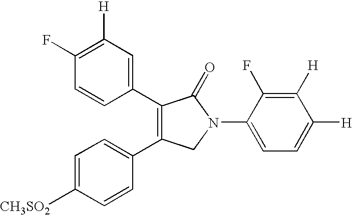 |
| B-245 | 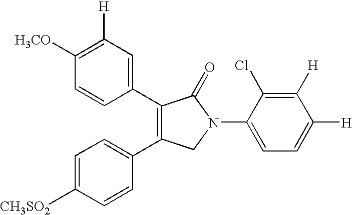 |
| B-246 | 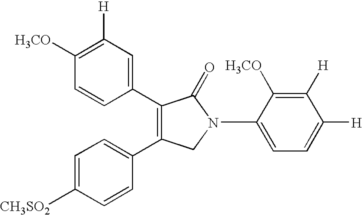 |
| B-247 | 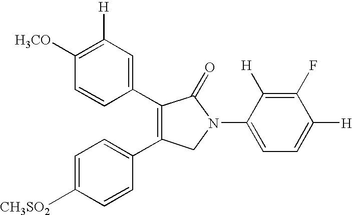 |
| B-248 | 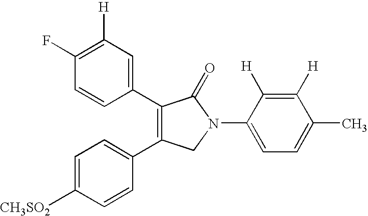 |
| B-249 | 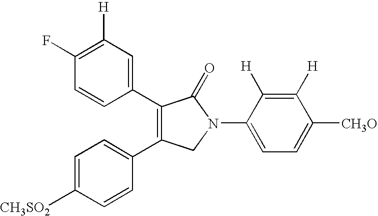 |
| B-250 | 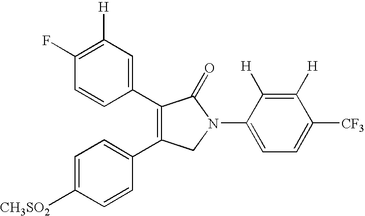 |
| B-251 | 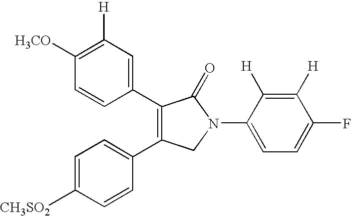 |
| B-252 | 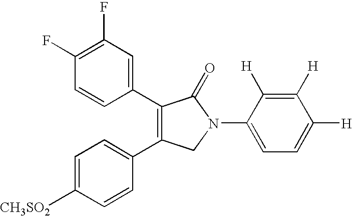 |

In yet another embodiment, the cyclooxygenase-2 selective inhibitor is other than BMS-279652 (CAS Registry No. 448263-62-5), BMS-279654 (CAS Registry No. 448263-63-6), and BMS-279655 (CAS Registry No. 448263-71-6). In an alternative of this embodiment, when the anti-herpes viral agent is an anti-HCMV agent, the cyclooxygenase-2 selective inhibitor is other than BMS-279652 (CAS Registry No. 448263-62-5), BMS-279654 (CAS Registry No. 448263-63-6), and BMS-279655 (CAS Registry No. 448263-71-6). In another alternative of this embodiment, when the anti-herpes viral agent is ganciclovir, the cyclooxygenase-2 selective inhibitor is other than BMS-279652 (CAS Registry No. 448263-62-5), BMS-279654 (CAS Registry No. 448263-63-6), and BMS-279655 (CAS Registry No. 448263-71-6).

The cyclooxygenase-2 selective inhibitor employed in the present invention can exist in tautomeric, geometric or stereoisomeric forms. Generally speaking, suitable cyclooxygenase-2 selective inhibitors that are in tautomeric, geometric or stereoisomeric forms are those compounds that inhibit cyclooxygenase-2 activity by about 25%, more typically by about 50%, and even more typically, by about 75% or more when present at a concentration of 100 μM or less. The present invention contemplates all such compounds, including cis- and trans-geometric isomers, E- and Z-geometric isomers, R— and S-enantiomers, diastereomers, d-isomers, 1-isomers, the racemic mixtures thereof and other mixtures thereof. Pharmaceutically acceptable salts of such tautomeric, geometric or stereoisomeric forms are also included within the invention. The terms “cis” and “trans”, as used herein, denote a form of geometric isomerism in which two carbon atoms connected by a double bond will each have a hydrogen atom on the same side of the double bond (“cis”) or on opposite sides of the double bond (“trans”). Some of the compounds described contain alkenyl groups, and are meant to include both cis and trans or “E” and “Z” geometric forms. Furthermore, some of the compounds described contain one or more stereocenters and are meant to include R, S, and mixtures or R and S forms for each stereocenter present.

The cyclooxygenase-2 selective inhibitors utilized in the present invention may be in the form of free bases or pharmaceutically acceptable acid addition salts thereof. The term “pharmaceutically-acceptable salts” are salts commonly used to form alkali metal salts and to form addition salts of free acids or free bases. The nature of the salt may vary, provided that it is pharmaceutically acceptable. Suitable pharmaceutically acceptable acid addition salts of compounds for use in the present methods may be prepared from an inorganic acid or from an organic acid. Examples of such inorganic acids are hydrochloric, hydrobromic, hydroiodic, nitric, carbonic, sulfuric and phosphoric acid. Appropriate organic acids may be selected from aliphatic, cycloaliphatic, aromatic, araliphatic, heterocyclic, carboxylic and sulfonic classes of organic acids, examples of which are formic, acetic, propionic, succinic, glycolic, gluconic, lactic, malic, tartaric, citric, ascorbic, glucuronic, maleic, fumaric, pyruvic, aspartic, glutamic, benzoic, anthranilic, mesylic, 4-hydroxybenzoic, phenylacetic, mandelic, embonic (pamoic), methanesulfonic, ethanesulfonic, benzenesulfonic, pantothenic, 2-hydroxyethanesulfonic, toluenesulfonic, sulfanilic, cyclohexylaminosulfonic, stearic, algenic, hydroxybutyric, salicylic, galactaric and galacturonic acid. Suitable pharmaceutically-acceptable base addition salts of compounds of use in the present methods include metallic salts made from aluminum, calcium, lithium, magnesium, potassium, sodium and zinc or organic salts made from N,N′-dibenzylethylenediamine, chloroprocaine, choline, diethanolamine, ethylenediamine, meglumine (N-methylglucamine) and procaine. All of these salts may be prepared by conventional means from the corresponding compound by reacting, for example, the appropriate acid or base with the compound of any Formula set forth herein.

The cyclooxygenase-2 selective inhibitors of the present invention can be formulated into pharmaceutical compositions and administered by a number of different means that will deliver a therapeutically effective dose. Such compositions can be administered orally, parenterally, by inhalation spray, rectally, intradermally, transdermally, or topically in dosage unit formulations containing conventional nontoxic pharmaceutically acceptable carriers, adjuvants, and vehicles as desired. Topical administration may also involve the use of transdermal administration such as transdermal patches or iontophoresis devices. The term parenteral as used herein includes subcutaneous, intravenous, intramuscular, or intrasternal injection, or infusion techniques. Formulation of drugs is discussed in, for example, Hoover, John E., Remington's Pharmaceutical Sciences, Mack Publishing Co., Easton, Pa. (1975), and Liberman, H. A. and Lachman, L., Eds., Pharmaceutical Dosage Forms, Marcel Decker, New York, N.Y. (1980).

Injectable preparations, for example, sterile injectable aqueous or oleaginous suspensions, can be formulated according to the known art using suitable dispersing or wetting agents and suspending agents. The sterile injectable preparation may also be a sterile injectable solution or suspension in a nontoxic parenterally acceptable diluent or solvent. Among the acceptable vehicles and solvents that may be employed are water, Ringer's solution, and isotonic sodium chloride solution. In addition, sterile, fixed oils are conventionally employed as a solvent or suspending medium. For this purpose, any bland fixed oil may be employed, including synthetic mono- or diglycerides. In addition, fatty acids such as oleic acid are useful in the preparation of injectables. Dimethyl acetamide, surfactants including ionic and non-ionic detergents, and polyethylene glycols can be used. Mixtures of solvents and wetting agents such as those discussed above are also useful.

Suppositories for rectal administration of the compounds discussed herein can be prepared by mixing the active agent with a suitable non-irritating excipient such as cocoa butter, synthetic mono-, di-, or triglycerides, fatty acids, or polyethylene glycols which are solid at ordinary temperatures but liquid at the rectal temperature, and which will therefore melt in the rectum and release the drug.

Solid dosage forms for oral administration may include capsules, tablets, pills, powders, and granules. In such solid dosage forms, the compounds are ordinarily combined with one or more adjuvants appropriate to the indicated route of administration. If administered per os, the compounds can be admixed with lactose, sucrose, starch powder, cellulose esters of alkanoic acids, cellulose alkyl esters, talc, stearic acid, magnesium stearate, magnesium oxide, sodium and calcium salts of phosphoric and sulfuric acids, gelatin, acacia gum, sodium alginate, polyvinylpyrrolidone, and/or polyvinyl alcohol, and then tableted or encapsulated for convenient administration. Such capsules or tablets can contain a controlled-release formulation as can be provided in a dispersion of active compound in hydroxypropylmethyl cellulose. In the case of capsules, tablets, and pills, the dosage forms can also comprise buffering agents such as sodium citrate, or magnesium or calcium carbonate or bicarbonate. Tablets and pills can additionally be prepared with enteric coatings.

For therapeutic purposes, formulations for parenteral administration can be in the form of aqueous or non-aqueous isotonic sterile injection solutions or suspensions. These solutions and suspensions can be prepared from sterile powders or granules having one or more of the carriers or diluents mentioned for use in the formulations for oral administration. The compounds can be dissolved in water, polyethylene glycol, propylene glycol, ethanol, corn oil, cottonseed oil, peanut oil, sesame oil, benzyl alcohol, sodium chloride, and/or various buffers. Other adjuvants and modes of administration are well and widely known in the pharmaceutical art.

Liquid dosage forms for oral administration can include pharmaceutically acceptable emulsions, solutions, suspensions, syrups, and elixirs containing inert diluents commonly used in the art, such as water. Such compositions can also comprise adjuvants, such as wetting agents, emulsifying and suspending agents, and sweetening, flavoring, and perfuming agents.

The amount of active ingredient that can be combined with the carrier materials to produce a single dosage of the cyclooxygenase-2 selective inhibitor will vary depending upon the patient and the particular mode of administration. In general, the pharmaceutical compositions may contain a cyclooxygenase-2 selective inhibitor in the range of about 0.1 to 2000 mg, more typically, in the range of about 0.5 to 500 mg and still more typically, between about 1 and 200 mg. A daily dose of about 0.01 to 100 mg/kg body weight, or more typically, between about 0.1 and about 50 mg/kg body weight and even more typically, from about 1 to 20 mg/kg body weight, may be appropriate. The daily dose is generally administered in one to about four doses per day.

In one embodiment, when the cyclooxygenase-2 selective inhibitor comprises rofecoxib, it is typical that the amount used is within a range of from about 0.15 to about 1.0 mg/day·kg, and even more typically, from about 0.18 to about 0.4 mg/day·kg.

In still another embodiment, when the cyclooxygenase-2 selective inhibitor comprises etoricoxib, it is typical that the amount used is within a range of from about 0.5 to about 5 mg/day·kg, and even more typically, from about 0.8 to about 4 mg/day·kg.

Further, when the cyclooxygenase-2 selective inhibitor comprises celecoxib, it is typical that the amount used is within a range of from about 1 to about 20 mg/day·kg, even more typically, from about 1.4 to about 8.6 mg/day·kg, and yet more typically, from about 2 to about 3 mg/day·kg.

When the cyclooxygenase-2 selective inhibitor comprises valdecoxib, it is typical that the amount used is within a range of from about 0.1 to about 5 mg/day·kg, and even more typically, from about 0.8 to about 4 mg/day·kg.

In a further embodiment, when the cyclooxygenase-2 selective inhibitor comprises parecoxib, it is typical that the amount used is within a range of from about 0.1 to about 5 mg/day·kg, and even more typically, from about 1 to about 3 mg/day·kg.

Those skilled in the art will appreciate that dosages may also be determined with guidance from Goodman & Goldman's The Pharmacological Basis of Therapeutics, Ninth Edition (1996), Appendix II, pp. 1707-1711 and from Goodman & Goldman's The Pharmacological Basis of Therapeutics, Tenth Edition (2001), Appendix II, pp. 475-493.

Anti-Herpes Virus Agents

In aspects of the invention related to combination therapy, the composition also comprises an anti-herpes virus agent that is not a cyclooxgyenase-2 selective inhibitor. A number of anti-herpes virus agents can be used in the current invention to the extent that the agent is capable of achieving viral inhibition by a mechanism other than inhibition of cyclooxgyenase-2. In general terms, such viral inhibition is a decrease in the severity of herpes virus infection as compared to that which would occur in the absence of the administration of the composition to the subject. This decrease in severity may result from a number of different factors including: a reduction in viral number, a reduction in viral replication, a reduction in the subject's cell growth infected with the virus, a reduction in cellular replication in the subject, a reduction in cellular mitosis in a subject, a reduction in viral colonization or any combination thereof. Generally speaking, the anti-herpes virus agents typically fall into one of two categories: agents that inhibit herpes virus infection by substantially inhibiting the herpes virus, or agents that help fight herpes virus naturally by stimulating the subject's immune response system. Suitable anti-herpes virus agents typically include viral cellular entry inhibitors, viral replication inhibitors, viral assembly inhibitors, human immune enhancing agents, virucidal agents, and antimitotic agents.

One aspect of the invention encompasses anti-herpes virus agents that are viral cellular entry inhibitors. Viral cellular entry inhibitors typically disrupt viral association with the subject's cell membrane thereby substantially inhibiting entry or release of the virus into the subject's cell. In one embodiment, the viral cellular inhibitor is a virion receptor/co receptor-binding antagonist. A number of different agents capable of disrupting herpes virus association with the subject's cell membrane may be employed. By way of example, heparan sulfate is a primary receptor for herpes virus fusion with the subject's cell. Very low doses of sodium heparin bind competitively with the subject's cell surface heparan sulfate receptors and thus, inhibit the very earliest stages of virion fusion. Other viral entry inhibitors known in the art may also be utilized in the practice of the invention.

Yet another aspect of the invention embraces anti-herpes virus agents that are viral replication inhibitors. Generally speaking, viral replication inhibitors substantially inhibit the synthesis of viral nucleic acid from which new virus particles are produced. In one embodiment, the viral replication inhibitor is a nucleoside analog that inhibits herpes virus DNA polymerase. By way of example, suitable nucleoside analogs for use in the current invention include 5-fluorodeoxyuridine (FUDR), 5-iododeoxyuridine, thymine arabinoside, ganciclovir, foscarnet, cidofovir, acycloguanosine and tri-fluorothymidine. In an alternative of this embodiment, the viral replication inhibitor is an anti-herpes virus drug that inhibits herpes virus DNA polymerase resulting in premature termination of the DNA chain and a reduction of viral replication. Suitable anti- herpes virus drugs include acyclovir, famciclovir, and valaciclovir.

In another embodiment, the viral replication inhibitor is an antisense therapy agent. These agents are typically unmodified or modified antisense oligonucleotides directed against various herpes virus RNA sequences that have been shown to inhibit viral replication, both in a sequence-specific and in a non-sequence specific manner. Because of their complementary, the agent binds to the herpes virus nucleic acid and thereby prevents its transcription. Of course the particular antisense oligonucleotides employed will vary considerably depending upon its intended target within the herpes virus genome and one skilled in the art can readily design appropriate antisense oligonucleotides for use in the present invention.

In still another embodiment, the viral replication inhibitor is glutathione (GSH) or selenium (Se²⁺). GSH and selenium interfere with the late replication stages of herpes virus and advantageously do not disturb normal cellular metabolism. By way of example, in vitro studies have shown that intracellular, endogenous, reduced GSH levels are significantly and immediately decreased in the first 24 hours after herpes virus invasion (Palamara, A T., et al., (1995) Antiviral Res. 27(3) 237-53). Supplementation with exogenous GSH not only restored intracellular levels almost to those found in uninfected cells, but also inhibited over 99% of the replication of herpes virus

A further aspect of the invention encompasses anti-herpes virus agents that inhibit or prevent assembly of the virus after its replication. Generally speaking, viral assembly inhibitors inhibit or prevent viral RNA processing, glycosylation, or capsid formation. In one embodiment, the inhibitor of viral assembly is a viral RNA process inhibitor. A number of suitable agents capable of blocking herpes virus RNA processing may be employed. In one alternative of this embodiment, the RNA processing inhibitor is a ribozyme. Ribozymes are RNA molecules having an enzymatic activity that are able to repeatedly cleave other separate RNA molecules in a nucleotide base sequence specific manner. Within the context of the present invention, the ribozyme employed typically cleaves herpes virus expressed RNA and in particular, viral mRNA targets, resulting in the destruction of mRNA transcript integrity. By way of example, the ribozyme employed is typically targeted to and prevents the translation of mRNAs encoding proteins required for viral genomic replication, virion structure, and viral infectivity, or maintenance of the latent state and therefore, interfere with critical events required for viral survival. Suitable ribozyme cleavage sites are at genes required for viral replication, e.g., protein synthesis, such as in the immediate early genes (ICP0, ICP4, ICP22 and ICP27), genes required for nucleic acid metabolism (UL13, 39, 40, 50), host shut-off (UL41), control of late viral protein synthesis, DNA replication (UL5, 8, 9, 29, 30, 42, 53) and structural protein encoding genes (gB and gC). Methods for selecting and constructing ribozymes suitable for use in the invention are more fully described in U.S. Pat. No. 6,440,719, which is hereby incorporated by reference in its entirety.

In another embodiment, the inhibitor of viral assembly is a glycosylation inhibitor. Certain herpes virus viral proteins undergo glycosylation, a step that is necessary for not only entry and replication of the virus, but also its assembly after replication. A number of agents capable of blocking herpes virus glycosylation may be employed. By way of example, one suitable herpes virus glycosylation inhibitor is 2-deoxy-D-glucose, which has been shown to retard the appearance of both HSV-1 and HSV-2. By way of further example, another suitable herpes virus glycosylation inhibitor is glucosamine, which has been shown to inhibit several steps in the metabolism of virus-induced cellular surface glycoproteins.

In yet another embodiment, the viral assembly inhibitor is a protease inhibitor. Protease inhibitors block the protease enzyme. Generally speaking, when new herpes virus particles break off from an infected cell, protease enzyme is employed to cut long protein strands into the parts required to assemble a mature virus. By inhibiting the protease enzyme, the necessary smaller-sized viral proteins cannot be made, and therefore, proper viral assembly cannot occur. As a result, the virus is prevented from spreading from cell to cell. A suitable such agent is one capable of inhibiting the herpes virus protease enzyme. By way of example, suitable protease inhibitor gene targets are more fully described in U.S. Pat. No. 6,410, 704 and U.S. Pat. No. 5,486,470, which are both hereby incorporated by reference in their entirety.

Another aspect of the invention encompasses anti-herpes virus agents that are human immune enhancing agents. Typically, human immune enhancing agents allow the body to slow the progression of herpes virus by substantially increasing the immune response of the subject. In one embodiment, the human immune enhancing agent is an antioxidant. In general terms, antioxidants aide in eliminating free radicals that are byproducts of a number of reactions that normally occur in the body. If left unchecked, these free radicals not only compromise cell membrane integrity, but also mediate several disease states including cancer and neurological disorders. Typically, herpes virus infection results in higher levels of free radical formation in the subject. The administration of antioxidants, without being bound to any particular theory, is believed to enhance the response of the subject against the virus by aiding in free radical elimination. Suitable agents for use as antioxidants are shown in Table N.

TABLE N

| Compound | |
|---|---|
| No. | Compound |
| N1 | 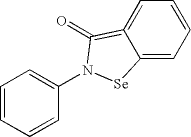 |
| | 2-phenyl-1,2-benzisoselenazol-3(2H)-one |
| N2 | 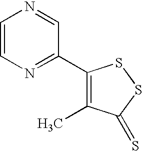 |
| | 4-methyl-5-(pyrazinyl)-3H-1,2-dithiole-3- |
| | thione |

In another embodiment, the human immune enhancing agent is an interferon. Interferons are members of a family of glycoproteins, classified as cytokines. Interferon, like several other cytokines, prevent viral replication as well as stimulate other aspects of the subject's own immune system to fight herpes virus infection. By way of example, one mechanism by which these agents stimulate a subject's immune system is that they bind to specific receptors on cell surfaces, and thereby initiate a cascade of events, including induction of specific proteins. These proteins in turn, stimulate antiviral, antiproliferative, and other actions that mediate immune response. An interferon that is effective in substantially preventing or inhibiting herpes virus infection may be employed. By way of example, suitable interferons for use in the present invention are shown in Table O.

TABLE O

| Com- | |
|---|---|
| pound | |
| No. | Compound |
| O1 | 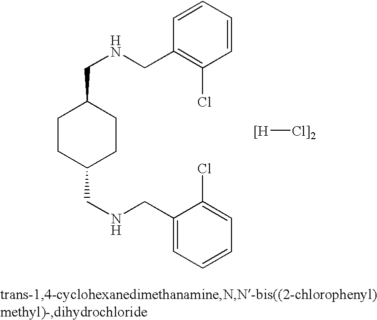 |
| O2 | 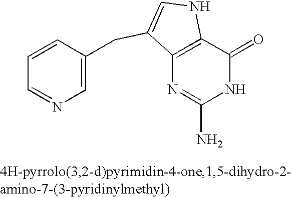 |
| O3 | 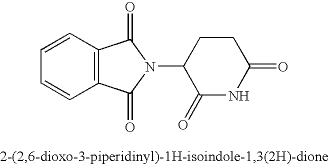 |

Another aspect of the invention encompasses anti-herpes virus agents that are natural products. A natural product that is effective in substantially preventing or inhibiting herpes virus infection may be employed. By way of example, suitable natural products are shown in Table P.

TABLE P

| | Active Ingredient |
|---|---|
| Herpasil by | Prunella Vulgaris, |
| SkinChoice | Melissa Officianalis, |
| | Calendula, Echinacea, Comfrey |
| Virostat | Viroxyn |
| Viraway | Herbs, essential and |
| | non-essential amino |
| | acids, B vitamins, |
| | folic acid, vitamin C, |
| | vitamin E, and protein Minerals |
| Phyto-Clear | Concentrated herb extracts |
| Simply Essential's | Myrrh, Peppermint, Eucalyptus, and |
| Herpe Alternative | Melissa, St. Johns Wort, Calendula, |
| | Arnica |
| Pure Lip Solution Pack | Zinc |
| Neuragen RL | Geranium Oil and other non-specified |
| | plant oils |
| Herpes Herbs | Chinese Herbs |
| Buddy's Oil | Vegetable Oils, Vitamin E |
| Herpe-Care | Chaparral |
| Attack Pack Gold | P. Vulgaris |
| Vir-L-Lysine | L-Lysine, Bee Pollen |
| Simplex Solutions & | Oriental and Western Botanicals |
| Immune Enhance Herbal | |
| System | |
| Herpes Prevention Formula | Chinese Herbs, Multivitamins |
| Progenn ® | (Unclear) |
| Let There Be Light | Fluorescent photo-therapy |
| Red Marine Algae | Dumontiaceae |
| Herpes No More | H-Balm |
| Herpeskil | Blended extracts of plants |
| Herpes-V BALM | Lithospermum root |
| Inter-Fear-On Magic | (Unclear) |
| Herpes Treatment by | Salicylic acid |
| Waldon Research | |
| Choraphor | Hypericum and Sulfur |

Another aspect of the invention encompasses anti-herpes virus agents that are antimitotic agents. Antimitotic agents typically inhibit or prevent mitosis or nuclear division of the subject's cell. Generally speaking, these agents slow viral replication and concomitantly, viral growth, by preventing division of a subject's cells infected with herpes virus.

In one embodiment, the antimitotic agent is podophyllotoxin. Podophyllotoxin selectively arrests mitosis in the metaphase stage of infected cutaneous cells, causing necrosis of the infected cells. The podophyllotoxin may be obtained from a number of sources. For example, in one embodiment, the podophyllotoxin may be obtained from a number of commercially available sources sold under tradenames such as podofilox (brand name “Condylox®” supplied by Oclassen Pharmaceuticals, Inc.), which is a glucoside extract synthesized chemically or purified from the plant families Coniferae and Berberidaceae. In yet another embodiment, the podophyllotoxin may be obtained from podphyllum resin (brand name “Pod-Ben-25” or “Podofin®”), which is a powdered mixture of resins removed from Podophyllum peltatum (more commonly known as the mayapple or American mandrake), a pereninial plant in the Berberidaceae family and found in the woodlands in Canada and the Eastern United States. In another embodiment, the antimitotic agents are oxygenated esters of 4-idodophenylamino benzhydroxamic acid or derivatives thereof as disclosed in WO/002062 13, which is hereby incorporated by reference in its entirety. These agents inhibit MAP kinase, which is an enzyme essential for cellular proliferation. Inhibition of this enzyme completely arrests mitogenesis.

A further aspect of the invention encompasses anti-herpes virus agents that are virucidal agents. Virucidal agents are competitive inhibitors of viral DNA polymerase. By way of example, in one embodiment, the virucidal agent is cidofovir. Cidofovir, (S)-1-(3-Hydroxy-2-phosphonylmethoxypropyl) cytosine (HPMPC), is an acyclic nucleoside phosphonate with broad-spectrum activity against a wide variety of DNA viruses, including herpes virus. The mechanism of action of cidofovir is based upon the interaction of its active intracellular metabolite, the diphosphorylated HPMPC derivative HPMPCpp, with the viral DNA polymerase. HPMPCpp has been shown to block DNA synthesis by DNA chain termination following incorporation of two consecutive HPMPC moledules at the 3′-end of the DNA chain. Cidofovir can be obtained from commercial sources. In addition, other compounds suitable for use as virucidal agents in the present invention are shown in Table Q.

TABLE Q

| Compound | |
|---|---|
| No. | Compound |
| Q1 | 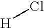 |
| | 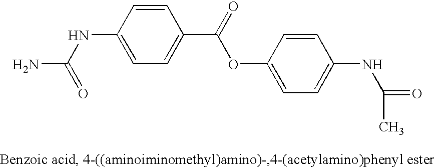 |
| Q2 | Cyanovirin-N |
| Q3 | 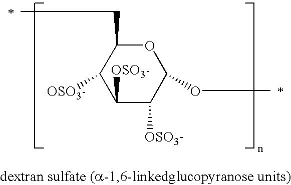 |
| Q4 | 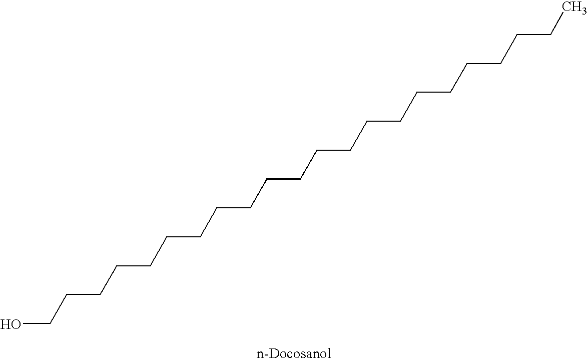 |
| Q5 | 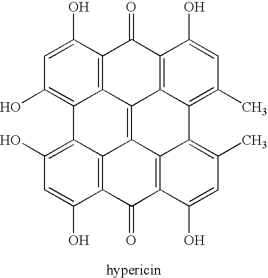 |
| Q6 | 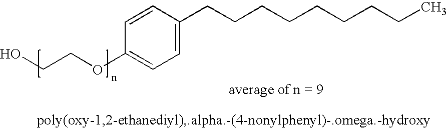 |
| Q7 | 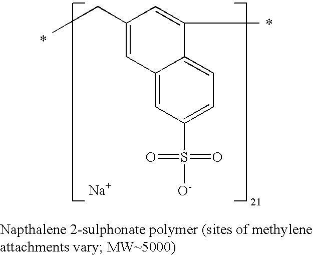 |
| Q8 | 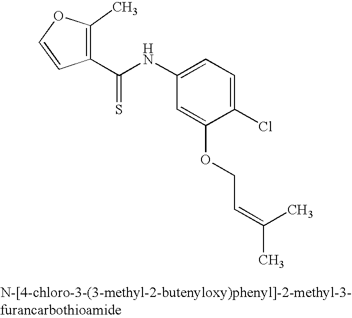 |

In yet a further aspect of the invention, the anti-herpes virus agent is an antineoplastic agent. These agents reduce cell proliferation and thus arrest the growth of new cells or tissue, which may be benign or malignant. Although historically employed as a chemotherapeutic agent, antineoplastic agents may be effective against herpes virus. In one embodiment, the antineoplastic agent is 5-fluorouracil. 5-Fluorouracil (Efudex®, Adrucil®, Fluoroplex®) interferes with DNA synthesis by blocking the methylation of deoxyuridylic acid and inhibits thymidylate syntheses, which subsequently reduces cell proliferation. In another embodiment, the antineoplastic agent is an oxygenated ester of 4-iodophenylamino benzhydroxamic acid. These compounds are further described in WO/0206213, which is hereby incorporated by reference in its entirety. In yet another alternative of this embodiment, the antineoplastic agent is bleomycin (brand name “Blenoxanee®”). In addition, other compounds suitable for use as antineoplastic agents in the present invention are shown in Table R.

TABLE R

| Com- | |
|---|---|
| pound | |
| No. | Compound |
| R1 | 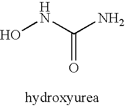 |
| R2 | 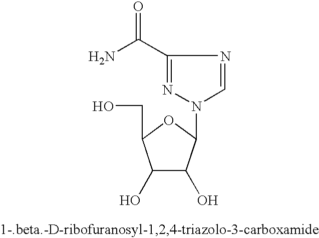 |
| R3 | 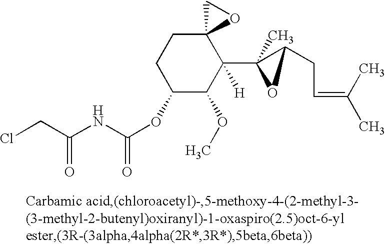 |

Of course, it will be apparent to those skilled in the art that it is possible, and perhaps desirable, to combine various classes of anti-herpes virus agents for use in the present invention. Accordingly, it is contemplated that any class of anti-herpes virus agent may be combined with one or more other classes to create a composition optimized for treating subjects having various stages of herpes virus progression. By way of example, one such composition may include an immune enhancing agent, a viral replication inhibitor, and a natural product. By way of further example, the composition may include a viral assembly inhibitor, and an interferon. A skilled artisan can readily design compositions having combinations of different classes of anti-herpes virus agents so as to optimize treatment for a particular subject.

Generally speaking, the pharmacokinetics of the particular agent to be administered will dictate the most preferred method of administration and dosing regiment. The anti-herpes virus agent can be administered as a pharmaceutical composition with or without a carrier. The terms “pharmaceutically acceptable carrier” or a “carrier” refer to any generally acceptable excipient or drug delivery composition that is relatively inert and non-toxic. Exemplary carriers include sterile water, salt solutions (such as Ringer's solution), alcohols, gelatin, talc, viscous paraffin, fatty acid esters, hydroxymethylcellulose, polyvinyl pyrolidone, calcium carbonate, carbohydrates (such as lactose, sucrose, dextrose, mannose, albumin, starch, cellulose, silica gel, polyethylene glycol (PEG), dried skim milk, rice flour, magnesium stearate, and the like. Suitable formulations and additional carriers are described in Remington's Pharmaceutical Sciences, (17.sup.th Ed., Mack Pub. Co., Easton, Pa.). Such preparations can be sterilized and, if desired, mixed with auxiliary agents, e.g., lubricants, preservatives, stabilizers, wetting agents, emulsifiers, salts for influencing osmotic pressure, buffers, coloring, preservatives and/or aromatic substances and the like which do not deleteriously react with the active compounds. Typical preservatives can include, potassium sorbate, sodium metabisulfite, methyl paraben, propyl paraben, thimerosal, etc. The composition can also be combined where desired with other active substances, e.g., enzyme inhibitors, to reduce metabolic degradation.

Moreover, the anti-herpes virus agent can be a liquid solution, suspension, emulsion, tablet, pill, capsule, sustained release formulation, or powder. The method of administration can dictate how the composition will be formulated. For example, the composition can be formulated as a suppository, with traditional binders and carriers such as triglycerides. Oral formulation can include standard carriers such as pharmaceutical grades of mannitol, lactose, starch, magnesium stearate, sodium saccharine, cellulose, or magnesium carbonate.

In another embodiment, the anti-herpes virus agent can be administered intravenously, parenterally, intramuscular, subcutaneously, orally, nasally, topically, by inhalation, by implant, by injection, or by suppository. For enteral or mucosal application (including via oral and nasal mucosa), particularly suitable are tablets, liquids, drops, suppositories or capsules. A syrup, elixir or the like can be used wherein a sweetened vehicle is employed. Liposomes, microspheres, and microcapsules are available and can be used. Pulmonary administration can be accomplished, for example, using any of various delivery devices known in the art such as an inhaler. See. e.g. S. P. Newman (1984) in Aerosols and the Lung, Clarke and Davis (eds.), Butterworths, London, England, pp. 197-224; PCT Publication No. WO 92/16192; PCT Publication No. WO 91/08760. For parenteral application, particularly suitable are injectable, sterile solutions, preferably oily or aqueous solutions, as well as suspensions, emulsions, or implants, including suppositories. In particular, carriers for parenteral administration include aqueous solutions of dextrose, saline, pure water, ethanol, glycerol, propylene glycol, peanut oil, sesame oil, polyoxyethylene-polyoxypropylene block polymers, and the like.

The actual effective amount of anti-herpes virus agent to be administered in the combination can and will vary according to the specific composition being utilized, the mode of administration and the age, weight and condition of the subject. Moreover, the amount administered will also vary considerably depending upon the particular syndrome of herpes virus infection being treated. By way of example, when herpes simplex virus is being treated, (HSV) three distinct syndromes are associated with genital herpes caused by HSV-2 infection: first-episode primary infection, first-episode nonprimary infection, and recurrent infections. First-episode primary infection occurs when a subject is antibody-negative to either strain of HSV. First-episode nonprimary infection occurs in subjects with preexisting antibodies, usually from previous oral HSV-1 infection. Nonprimary episodes produce symptoms that are often milder and with fewer lesions that are less painful and shorter in duration, commonly only lasting a week. Recurrent infections produce milder symptoms localized to the genital area and depending upon the particular subject, may occur from 1 to several times per year for the lifetime of the subject. Although the precise cause for recurrence has not been fully characterized, it is often triggered by menstruation, stress, sunlight, surgical trauma, or excessive genital friction.

In one aspect of the invention, the composition is administered to treat a first episode primary or nonprimary infection. By way of example, in one embodiment, when the anti-herpes virus agent is acyclovir, it is typical that the amount used is approximately 500 to about 2000 milligrams per day for seven to ten days and more typically, about 1200 milligrams per day for seven to ten days. In one alternative of this embodiment, when the anti-herpes virus agent is famciclovir, it is typical that the amount used is approximately 500 to about 1500 milligrams per day for seven to ten days and even more commonly, about 750 milligrams per day for seven to ten days. In yet another alternative of this embodiment, when the anti-herpes virus agent is valacycloivir, it is typical that the amount used is approximately 1 to about 3 grams per day for seven to ten days and even more commonly, about 2 grams per day for seven to ten days. Table 1A below provides a comparison of some commonly employed dosing regiments when the anti-herpes virus agent is acyclovir, famciclovir, or valacycloivir administered to a subject with either a first episode primary or nonprimary infection.

TABLE 1A
First-Epidsode Primary/Nonprimary

| Anti-Herpes | |
|---|---|
| Virus Agent | Dosing Regiment |
| Acyclovir | 400 milligrams orally 3 |
| | times a day for 7-10 days |
| Acyclovir | 200 milligrams orally 5 |
| | times a day for 7-10 days |
| Famciclovir | 250 milligrams orally 3 |
| | times a day for 7-10 days |
| Valacyclovir | 1 grams orally twice a |
| | day for 7-10 days |

Yet another aspect of the invention embraces use of the composition to treat recurrent infections. Generally speaking, typically two different treatment regimes are employed for the treatment of recurrent infections, episodic treatment and suppressive treatment. In one embodiment, the composition is administered as a part of an episodic treatment regime. Episodic treatment of genital herpes involves treating the disease when it occurs on an ad-hoc basis. Typically, taking the composition at the first signs of an episode will help reduce the length and severity of symptoms. By way of example, in one embodiment, when the anti-herpes virus agent is acyclovir, it is typical that the amount used is approximately 500 to about 2000 milligrams per day for five days and more typically, about 1000 milligrams per day for five days. In one alternative of this embodiment, when the anti-herpes virus agent is famciclovir, it is typical that the amount used is approximately 100 to about 500 milligrams per day for five days and even more commonly, about 250 milligrams per day for five days. In yet another alternative of this embodiment, when the anti-herpes virus agent is valacycloivir, it is typical that the amount used is approximately 500 milligrams to about 1500 milligrams per day for three days and even more commonly, about 1000 milligrams per day for three days. Table 1B below provides a comparison of some commonly employed dosing regiments when the anti-herpes virus agent is acyclovir, famciclovir, or valacycloivir administered to a subject as a part of an episodic treatment regime.

TABLE 1B
Episodic Therapy

| Anti-Herpes | |
|---|---|
| Virus Agent | Dosing Regiment |
| Acyclovir | 400 milligrams orally 3 times |
| | a day for 5 days |
| Acyclovir | 200 milligrams orally 5 times |
| | a day for 5 days |
| Acyclovir | 800 milligrams orally twice a |
| | day for 5 days |
| Famciclovir | 125 milligrams orally twice a |
| | day for 5 days |
| Valacyclovir | 500 milligrams orally twice a |
| | day for 3-5 days |
| Valacyclovir | 1 grams orally once a day for |
| | 5 days |

In yet another embodiment, the composition of the invention is administered as part of a suppressive treatment regime. Suppressive treatment involves taking medication on a daily basis for the lifetime of the subject to reduce the chance of an episode occurring. Typically, only subjects having either frequent (e.g. about 3 or more on a yearly basis) or particularly severe recurrent infections participate in a suppressive treatment regime. By way of example, in one embodiment, when the anti-herpes virus agent is acyclovir, it is typical that the amount used is approximately 500 to about 1000 milligrams per day and more typically, about 800 milligrams per day. In one alternative of this embodiment, when the anti-herpes virus agent is famciclovir, it is typical that the amount used is approximately 100 to about 500 milligrams per day and even more commonly, about 250 milligrams per day. In yet another alternative of this embodiment, when the anti-herpes virus agent is valacycloivir, it is typical that the amount used is approximately 500 milligrams to about 1500 milligrams per day and even more commonly, about 1000 milligrams per days. Table 1C below provides a comparison of some commonly employed dosing regiments when the anti-herpes virus agent is acyclovir, famciclovir, or valacycloivir administered to a subject as a part of a suppressive treatment regime.

TABLE 1C
Suppressive Therapy

| Anti-Herpes | |
|---|---|
| Virus Agent | Dosing Regiment |
| Acyclovir | 400 milligrams orally |
| | twice a day |
| Famciclovir | 250 milligrams orally |
| | twice a day |
| Valacyclovir | 500 milligrams orally |
| | once a day |
| Valacyclovir | 1 grams orally once a |
| | day |

Those skilled in the art will appreciate that dosages may also be determined with guidance from Goodman & Goldman's The Pharmacological Basis of Therapeutics, Ninth Edition (1996), Appendix II, pp. 1707-1711 and from Goodman & Goldman's The Pharmacological Basis of Therapeutics, Tenth Edition (2001), Appendix II, pp. 475-493.

The timing of the administration of the cyclooxygenase-2 selective inhibitor in relation to the administration of the anti-herpes virus agent may vary from subject to subject and depend upon the stage of infection being treated. In one embodiment of the invention, the cyclooxygenase-2 selective inhibitor and anti-herpes virus agent may be administered substantially simultaneously, meaning that both agents may be administered to the subject at approximately the same time. For example, the cyclooxygenase-2 selective inhibitor or pharmaceutically acceptable salt or prodrug thereof is administered during a continuous period beginning on the same day as the beginning of the anti-herpes virus agent and extending to a period after the end of the anti-herpes virus agent. Alternatively, the cyclooxygenase-2 selective inhibitor and anti-herpes virus agent may be administered sequentially, meaning that they are administered at separate times during separate treatments. In one embodiment, for example, the cyclooxygenase-2 selective inhibitor is administered during a continuous period beginning prior to administration of the anti-herpes virus agent and ending after administration of the anti-herpes virus agent. Of course, it is also possible that the cyclooxygenase-2 selective inhibitor may be administered either more or less frequently than the anti-herpes virus agent. One skilled in the art can readily design suitable treatment regiments for a particular subject depending on the particular stage of herpes virus infection being treated. Moreover, it will be apparent to those skilled in the art that it is possible, and perhaps desirable, to combine various times and methods of administration in the practice of the present invention.

Combination Therapies

Generally speaking, it is contemplated that the composition employed in the practice of the invention may include one or more of any of the cyclooxygenase-2 selective inhibitors detailed above in combination with one or more of any of the anti-herpes virus agents detailed above. By way of a non limiting example, Table C1 details a number of suitable combinations that are useful in the methods and compositions of the current invention. The combination may also include an isomer, a pharmaceutically acceptable salt, ester, or prodrug of any of the cyclooxygenase-2 selective inhibitors or anti-herpes virus agents listed in Table C1.

TABLE NO. C1

| Cyclooxygenase-2 | Anti-Herpes |
|---|---|
| Selective Inhibitor | Virus Agents |
| a compound having | ganciclovir |
| formula I | |
| a compound having | foscarnet |
| formula I | |
| a compound having | cidofovir |
| formula I | |
| a compound having | acycloguanosine |
| formula I | |
| a compound having | tri-fluorothymidine |
| formula I | |
| a compound having | acyclovir |
| formula I | |
| a compound having | famciclovir |
| formula I | |
| a compound having | valaciclovir |
| formula I | |
| a compound having | ganciclovir |
| formula II | |
| a compound having | foscarnet |
| formula II | |
| a compound having | cidofovir |
| formula II | |
| a compound having | acycloguanosine |
| formula II | |
| a compound having | tri-fluorothymidine |
| formula II | |
| a compound having | acyclovir |
| formula II | |
| a compound having | famciclovir |
| formula II | |
| a compound having | valaciclovir |
| formula II | |
| a compound having | ganciclovir |
| formula III | |
| a compound having | foscarnet |
| formula III | |
| a compound having | cidofovir |
| formula III | |
| a compound having | acycloguanosine |
| formula III | |
| a compound having | tri-fluorothymidine |
| formula III | |
| a compound having | acyclovir |
| formula III | |
| a compound having | famciclovir |
| formula III | |
| a compound having | valaciclovir |
| formula III | |
| a compound having | ganciclovir |
| formula VI | |
| a compound having | foscarnet |
| formula VI | |
| a compound having | cidofovir |
| formula VI | |
| a compound having | acycloguanosine |
| formula VI | |
| a compound having | tri-fluorothymidine |
| formula VI | |
| a compound having | acyclovir |
| formula VI | |
| a compound having | famciclovir |
| formula VI | |
| a compound having | valaciclovir |
| formula VI | |
| a compound having | ganciclovir |
| formula V | |
| a compound having | foscarnet |
| formula V | |
| a compound having | cidofovir |
| formula V | |
| a compound having | acycloguanosine |
| formula V | |
| a compound having | tri-fluorothymidine |
| formula V | |
| a compound having | acyclovir |
| formula V | |
| a compound having | famciclovir |
| formula V | |
| a compound having | valaciclovir |
| formula V | |

By way of further example, Table C2 details a number of suitable combinations that may be employed in the methods and compositions of the present invention. The combination may also include an isomer, a pharmaceutically acceptable salt, ester, or prodrug of any of the cyclooxygenase-2 selective inhibitors or anti-herpes virus agents listed in Table C2.

TABLE NO. C2

| Cyclooxygenase-2 | |
|---|---|
| Selective Inhibitor | Anti-Herpes Virus Agents |
| a compound selected from | ganciclovir |
| the group consisting of | |
| B-1, B-2, B-3, B-4, B-5, | |
| B-6, B-7, B-8, B-9, B-10, | |
| B-11, B-12, B-13, B-14, B-15, | |
| B-16, B-17, B-18, B-19, B-20, | |
| B-21, B-22, B-23, B-24, B-25, | |
| B-26, B-27, B-28, B-29, B-30, | |
| B-31, B-32, B-33, B-34, B-35, | |
| B-36, B-37, B-38, B-39, B-40, | |
| B-41, B-42, B-43, B-44, B-45, | |
| B-46, B-47, B-48, B-49, B-50, | |
| B-51, B-52, B-53, B-54, B-55, | |
| B-56, B-57, B-58, B-59, B-60, | |
| B-61, B-62, B-63, B-64, B-65, | |
| B-66, B-67, B-68, B-69, B-70, | |
| B-71, B-72, B-73, B-74, B-75, | |
| B-76, B-77, B-78, B-79, B-80, | |
| B-81, B-82, B-83, B-84, B-85, | |
| B-86, B-87, B-88, B-89, B-90, | |
| B-91, B-92, B-93, B-94, B-95, | |
| B-96, B-97, B-98, B-99, B-100, | |
| B-101, B-102, B-103, B-104, B-105, | |
| B-106, B-107, B-108, B-109, B-110, | |
| B-111, B-112, B-113, B-114, B-115, | |
| B-116, B-117, B-118, B-119, B-120, | |
| B-121, B-122, B-123, B-124, B-125, | |
| B-126, B-127, B-128, B-129, B-130, | |
| B-131, B-132, B-133, B-134, B-135, | |
| B-136, B-137, B-138, B-139, B-140, | |
| B-141, B-142, B-143, B-144, B-145, | |
| B-146, B-147, B-148, B-149, B-150, | |
| B-151, B-152, B-153, B-154, B-155, | |
| B-156, B-157, B-158, B-159, B-160, | |
| B-161, B-162, B-163, B-164, B-165, | |
| B-166, B-167, B-168, B-169, B-170, | |
| B-171, B-172, B-173, B-174, B-175, | |
| B-176, B-177, B-178, B-179, B-180, | |
| B-181, B-182, B-183, B-184, B-185, | |
| B-186, B-187, B-188, B-189, B-190, | |
| B-191, B-192, B-193, B-194, B-195, | |
| B-196, B-197, B-198, B-199, B-200, | |
| B-201, B-202, B-203, B-204, B-205, | |
| B-206, B-207, B-208, B-209, B-210, | |
| B-211, B-212, B-213, B-214, B-215, | |
| B-216, B-217, B-218, B-219, B-220, | |
| B-221, B-222, B-223, B-224, B-225, | |
| B-226, B-227, B-228, B-229, B-230, | |
| B-231, B-232,B233, B-234, B-235, | |
| B-236, B-237, B-238, B-239, B-240, | |
| B-241, B-242, B-243B-244, B-245, | |
| B-246, B-247, B-248, B-249, B-250, | |
| B-251, B-252 | |
| a compound selected from | foscarnet |
| the group consisting of | |
| B-1, B-2, B-3, B-4, B-5, | |
| B-6, B-7, B-8, B-9, B-10, | |
| B-11, B-12, B-13, B-14, B-15, | |
| B-16, B-17, B-18, B-19, B-20, | |
| B-21, B-22, B-23, B-24, B-25, | |
| B-26, B-27, B-28, B-29, B-30, | |
| B-31, B-32, B-33, B-34, B-35, | |
| B-36, B-37, B-38, B-39, B-40, | |
| B-41, B-42, B-43, B-44, B-45, | |
| B-46, B-47, B-48, B-49, B-50, | |
| B-51, B-52, B-53, B-54, B-55, | |
| B-56, B-57, B-58, B-59, B-60, | |
| B-61, B-62, B-63, B-64, B-65, | |
| B-66, B-67, B-68, B-69, B-70, | |
| B-71, B-72, B-73, B-74, B-75, | |
| B-76, B-77, B-78, B-79, B-80, | |
| B-81, B-82, B-83, B-84, B-85, | |
| B-86, B-87, B-88, B-89, B-90, | |
| B-91, B-92, B-93, B-94, B-95, | |
| B-96, B-97, B-98, B-99, B-100, | |
| B-101, B-102, B-103, B-104, B-105, | |
| B-106, B-107, B-108, B-109, B-110, | |
| B-111, B-112, B-113, B-114, B-115, | |
| B-116, B-117, B-118, B-119, B-120, | |
| B-121, B-122, B-123, B-124, B-125, | |
| B-126, B-127, B-128, B-129, B-130, | |
| B-131, B-132, B-133, B-134, B-135, | |
| B-136, B-137, B-138, B-139, B-140, | |
| B-141, B-142, B-143, B-144, B-145, | |
| B-146, B-147, B-148, B-149, B-150, | |
| B-151, B-152, B-153, B-154, B-155, | |
| B-156, B-157, B-158, B-159, B-160, | |
| B-161, B-162, B-163, B-164, B-165, | |
| B-166, B-167, B-168, B-169, B-170, | |
| B-171, B-172, B-173, B-174, B-175, | |
| B-176, B-177, B-178, B-179, B-180, | |
| B-181, B-182, B-183, B-184, B-185, | |
| B-186, B-187, B-188, B-189, B-190, | |
| B-191, B-192, B-193, B-194, B-195, | |
| B-196, B-197, B-198, B-199, B-200, | |
| B-201, B-202, B-203, B-204, B-205, | |
| B-206, B-207, B-208, B-209, B-210, | |
| B-211, B-212, B-213, B-214, B-215, | |
| B-216, B-217, B-218, B-219, B-220, | |
| B-221, B-222, B-223, B-224, B-225, | |
| B-226, B-227, B-228, B-229, B-230, | |
| B-231, B-232,B233, B-234, B-235, | |
| B-236, B-237, B-238, B-239, B-240, | |
| B-241, B-242, B-243B-244, B-245, | |
| B-246, B-247, B-248, B-249, B-250, | |
| B-251, B-252 | |
| a compound selected from | cidofovir |
| the group consisting of | |
| B-1, B-2, B-3, B-4, B-5, | |
| B-6, B-7, B-8, B-9, B-10, | |
| B-11, B-12, B-13, B-14, B-15, | |
| B-16, B-17, B-18, B-19, B-20, | |
| B-21, B-22, B-23, B-24, B-25, | |
| B-26, B-27, B-28, B-29, B-30, | |
| B-31, B-32, B-33, B-34, B-35, | |
| B-36, B-37, B-38, B-39, B-40, | |
| B-41, B-42, B-43, B-44, B-45, | |
| B-46, B-47, B-48, B-49, B-50, | |
| B-51, B-52, B-53, B-54, B-55, | |
| B-56, B-57, B-58, B-59, B-60, | |
| B-61, B-62, B-63, B-64, B-65, | |
| B-66, B-67, B-68, B-69, B-70, | |
| B-71, B-72, B-73, B-74, B-75, | |
| B-76, B-77, B-78, B-79, B-80, | |
| B-81, B-82, B-83, B-84, B-85, | |
| B-86, B-87, B-88, B-89, B-90, | |
| B-91, B-92, B-93, B-94, B-95, | |
| B-96, B-97, B-98, B-99, B-100, | |
| B-101, B-102, B-103, B-104, B-105, | |
| B-106, B-107, B-108, B-109, B-110, | |
| B-111, B-112, B-113, B-114, B-115, | |
| B-116, B-117, B-118, B-119, B-120, | |
| B-121, B-122, B-123, B-124, B-125, | |
| B-126, B-127, B-128, B-129, B-130, | |
| B-131, B-132, B-133, B-134, B-135, | |
| B-136, B-137, B-138, B-139, B-140, | |
| B-141, B-142, B-143, B-144, B-145, | |
| B-146, B-147, B-148, B-149, B-150, | |
| B-151, B-152, B-153, B-154, B-155, | |
| B-156, B-157, B-158, B-159, B-160, | |
| B-161, B-162, B-163, B-164, B-165, | |
| B-166, B-167, B-168, B-169, B-170, | |
| B-171, B-172, B-173, B-174, B-175, | |
| B-176, B-177, B-178, B-179, B-180, | |
| B-181, B-182, B-183, B-184, B-185, | |
| B-186, B-187, B-188, B-189, B-190, | |
| B-191, B-192, B-193, B-194, B-195, | |
| B-196, B-197, B-198, B-199, B-200, | |
| B-201, B-202, B-203, B-204, B-205, | |
| B-206, B-207, B-208, B-209, B-210, | |
| B-211, B-212, B-213, B-214, B-215, | |
| B-216, B-217, B-218, B-219, B-220, | |
| B-221, B-222, B-223, B-224, B-225, | |
| B-226, B-227, B-228, B-229, B-230, | |
| B-231, B-232,B233, B-234, B-235, | |
| B-236, B-237, B-238, B-239, B-240, | |
| B-241, B-242, B-243B-244, B-245, | |
| B-246, B-247, B-248, B-249, B-250, | |
| B-251, B-252 | |
| a compound selected from | acycloguanosine |
| the group consisting of | |
| B-1, B-2, B-3, B-4, B-5, | |
| B-6, B-7, B-8, B-9, B-10, | |
| B-11, B-12, B-13, B-14, B-15, | |
| B-16, B-17, B-18, B-19, B-20, | |
| B-21, B-22, B-23, B-24, B-25, | |
| B-26, B-27, B-28, B-29, B-30, | |
| B-31, B-32, B-33, B-34, B-35, | |
| B-36, B-37, B-38, B-39, B-40, | |
| B-41, B-42, B-43, B-44, B-45, | |
| B-46, B-47, B-48, B-49, B-50, | |
| B-51, B-52, B-53, B-54, B-55, | |
| B-56, B-57, B-58, B-59, B-60, | |
| B-61, B-62, B-63, B-64, B-65, | |
| B-66, B-67, B-68, B-69, B-70, | |
| B-71, B-72, B-73, B-74, B-75, | |
| B-76, B-77, B-78, B-79, B-80, | |
| B-81, B-82, B-83, B-84, B-85, | |
| B-86, B-87, B-88, B-89, B-90, | |
| B-91, B-92, B-93, B-94, B-95, | |
| B-96, B-97, B-98, B-99, B-100, | |
| B-101, B-102, B-103, B-104, B-105, | |
| B-106, B-107, B-108, B-109, B-110, | |
| B-111, B-112, B-113, B-114, B-115, | |
| B-116, B-117, B-118, B-119, B-120, | |
| B-121, B-122, B-123, B-124, B-125, | |
| B-126, B-127, B-128, B-129, B-130, | |
| B-131, B-132, B-133, B-134, B-135, | |
| B-136, B-137, B-138, B-139, B-140, | |
| B-141, B-142, B-143, B-144, B-145, | |
| B-146, B-147, B-148, B-149, B-150, | |
| B-151, B-152, B-153, B-154, B-155, | |
| B-156, B-157, B-158, B-159, B-160, | |
| B-161, B-162, B-163, B-164, B-165, | |
| B-166, B-167, B-168, B-169, B-170, | |
| B-171, B-172, B-173, B-174, B-175, | |
| B-176, B-177, B-178, B-179, B-180, | |
| B-181, B-182, B-183, B-184, B-185, | |
| B-186, B-187, B-188, B-189, B-190, | |
| B-191, B-192, B-193, B-194, B-195, | |
| B-196, B-197, B-198, B-199, B-200, | |
| B-201, B-202, B-203, B-204, B-205, | |
| B-206, B-207, B-208, B-209, B-210, | |
| B-211, B-212, B-213, B-214, B-215, | |
| B-216, B-217, B-218, B-219, B-220, | |
| B-221, B-222, B-223, B-224, B-225, | |
| B-226, B-227, B-228, B-229, B-230, | |
| B-231, B-232,B233, B-234, B-235, | |
| B-236, B-237, B-238, B-239, B-240, | |
| B-241, B-242, B-243B-244, B-245, | |
| B-246, B-247, B-248, B-249, B-250, | |
| B-251, B-252 | |
| a compound selected from | tri-fluorothymidine |
| the group consisting of | |
| B-1, B-2, B-3, B-4, B-5, | |
| B-6, B-7, B-8, B-9, B-10, | |
| B-11, B-12, B-13, B-14, B-15, | |
| B-16, B-17, B-18, B-19, B-20, | |
| B-21, B-22, B-23, B-24, B-25, | |
| B-26, B-27, B-28, B-29, B-30, | |
| B-31, B-32, B-33, B-34, B-35, | |
| B-36, B-37, B-38, B-39, B-40, | |
| B-41, B-42, B-43, B-44, B-45, | |
| B-46, B-47, B-48, B-49, B-50, | |
| B-51, B-52, B-53, B-54, B-55, | |
| B-56, B-57, B-58, B-59, B-60, | |
| B-61, B-62, B-63, B-64, B-65, | |
| B-66, B-67, B-68, B-69, B-70, | |
| B-71, B-72, B-73, B-74, B-75, | |
| B-76, B-77, B-78, B-79, B-80, | |
| B-81, B-82, B-83, B-84, B-85, | |
| B-86, B-87, B-88, B-89, B-90, | |
| B-91, B-92, B-93, B-94, B-95, | |
| B-96, B-97, B-98, B-99, B-100, | |
| B-101, B-102, B-103, B-104, B-105, | |
| B-106, B-107, B-108, B-109, B-110, | |
| B-111, B-112, B-113, B-114, B-115, | |
| B-116, B-117, B-118, B-119, B-120, | |
| B-121, B-122, B-123, B-124, B-125, | |
| B-126, B-127, B-128, B-129, B-130, | |
| B-131, B-132, B-133, B-134, B-135, | |
| B-136, B-137, B-138, B-139, B-140, | |
| B-141, B-142, B-143, B-144, B-145, | |
| B-146, B-147, B-148, B-149, B-150, | |
| B-151, B-152, B-153, B-154, B-155, | |
| B-156, B-157, B-158, B-159, B-160, | |
| B-161, B-162, B-163, B-164, B-165, | |
| B-166, B-167, B-168, B-169, B-170, | |
| B-171, B-172, B-173, B-174, B-175, | |
| B-176, B-177, B-178, B-179, B-180, | |
| B-181, B-182, B-183, B-184, B-185, | |
| B-186, B-187, B-188, B-189, B-190, | |
| B-191, B-192, B-193, B-194, B-195, | |
| B-196, B-197, B-198, B-199, B-200, | |
| B-201, B-202, B-203, B-204, B-205, | |
| B-206, B-207, B-208, B-209, B-210, | |
| B-211, B-212, B-213, B-214, B-215, | |
| B-216, B-217, B-218, B-219, B-220, | |
| B-221, B-222, B-223, B-224, B-225, | |
| B-226, B-227, B-228, B-229, B-230, | |
| B-231, B-232,B233, B-234, B-235, | |
| B-236, B-237, B-238, B-239, B-240, | |
| B-241, B-242, B-243B-244, B-245, | |
| B-246, B-247, B-248, B-249, B-250, | |
| B-251, B-252 | |
| a compound selected from | acyclovir |
| the group consisting of | |
| B-1, B-2, B-3, B-4, B-5, | |
| B-6, B-7, B-8, B-9, B-10, | |
| B-11, B-12, B-13, B-14, B-15, | |
| B-16, B-17, B-18, B-19, B-20, | |
| B-21, B-22, B-23, B-24, B-25, | |
| B-26, B-27, B-28, B-29, B-30, | |
| B-31, B-32, B-33, B-34, B-35, | |
| B-36, B-37, B-38, B-39, B-40, | |
| B-41, B-42, B-43, B-44, B-45, | |
| B-46, B-47, B-48, B-49, B-50, | |
| B-51, B-52, B-53, B-54, B-55, | |
| B-56, B-57, B-58, B-59, B-60, | |
| B-61, B-62, B-63, B-64, B-65, | |
| B-66, B-67, B-68, B-69, B-70, | |
| B-71, B-72, B-73, B-74, B-75, | |
| B-76, B-77, B-78, B-79, B-80, | |
| B-81, B-82, B-83, B-84, B-85, | |
| B-86, B-87, B-88, B-89, B-90, | |
| B-91, B-92, B-93, B-94, B-95, | |
| B-96, B-97, B-98, B-99, B-100, | |
| B-101, B-102, B-103, B-104, B-105, | |
| B-106, B-107, B-108, B-109, B-110, | |
| B-111, B-112, B-113, B-114, B-115, | |
| B-116, B-117, B-118, B-119, B-120, | |
| B-121, B-122, B-123, B-124, B-125, | |
| B-126, B-127, B-128, B-129, B-130, | |
| B-131, B-132, B-133, B-134, B-135, | |
| B-136, B-137, B-138, B-139, B-140, | |
| B-141, B-142, B-143, B-144, B-145, | |
| B-146, B-147, B-148, B-149, B-150, | |
| B-151, B-152, B-153, B-154, B-155, | |
| B-156, B-157, B-158, B-159, B-160, | |
| B-161, B-162, B-163, B-164, B-165, | |
| B-166, B-167, B-168, B-169, B-170, | |
| B-171, B-172, B-173, B-174, B-175, | |
| B-176, B-177, B-178, B-179, B-180, | |
| B-181, B-182, B-183, B-184, B-185, | |
| B-186, B-187, B-188, B-189, B-190, | |
| B-191, B-192, B-193, B-194, B-195, | |
| B-196, B-197, B-198, B-199, B-200, | |
| B-201, B-202, B-203, B-204, B-205, | |
| B-206, B-207, B-208, B-209, B-210, | |
| B-211, B-212, B-213, B-214, B-215, | |
| B-216, B-217, B-218, B-219, B-220, | |
| B-221, B-222, B-223, B-224, B-225, | |
| B-226, B-227, B-228, B-229, B-230, | |
| B-231, B-232,B233, B-234, B-235, | |
| B-236, B-237, B-238, B-239, B-240, | |
| B-241, B-242, B-243B-244, B-245, | |
| B-246, B-247, B-248, B-249, B-250, | |
| B-251, B-252 | |
| a compound selected from | famciclovir |
| the group consisting of | |
| B-1, B-2, B-3, B-4, B-5, | |
| B-6, B-7, B-8, B-9, B-10, | |
| B-11, B-12, B-13, B-14, B-15, | |
| B-16, B-17, B-18, B-19, B-20, | |
| B-21, B-22, B-23, B-24, B-25, | |
| B-26, B-27, B-28, B-29, B-30, | |
| B-31, B-32, B-33, B-34, B-35, | |
| B-36, B-37, B-38, B-39, B-40, | |
| B-41, B-42, B-43, B-44, B-45, | |
| B-46, B-47, B-48, B-49, B-50, | |
| B-51, B-52, B-53, B-54, B-55, | |
| B-56, B-57, B-58, B-59, B-60, | |
| B-61, B-62, B-63, B-64, B-65, | |
| B-66, B-67, B-68, B-69, B-70, | |
| B-71, B-72, B-73, B-74, B-75, | |
| B-76, B-77, B-78, B-79, B-80, | |
| B-81, B-82, B-83, B-84, B-85, | |
| B-86, B-87, B-88, B-89, B-90, | |
| B-91, B-92, B-93, B-94, B-95, | |
| B-96, B-97, B-98, B-99, B-100, | |
| B-101, B-102, B-103, B-104, B-105, | |
| B-106, B-107, B-108, B-109, B-110, | |
| B-111, B-112, B-113, B-114, B-115, | |
| B-116, B-117, B-118, B-119, B-120, | |
| B-121, B-122, B-123, B-124, B-125, | |
| B-126, B-127, B-128, B-129, B-130, | |
| B-131, B-132, B-133, B-134, B-135, | |
| B-136, B-137, B-138, B-139, B-140, | |
| B-141, B-142, B-143, B-144, B-145, | |
| B-146, B-147, B-148, B-149, B-150, | |
| B-151, B-152, B-153, B-154, B-155, | |
| B-156, B-157, B-158, B-159, B-160, | |
| B-161, B-162, B-163, B-164, B-165, | |
| B-166, B-167, B-168, B-169, B-170, | |
| B-171, B-172, B-173, B-174, B-175, | |
| B-176, B-177, B-178, B-179, B-180, | |
| B-181, B-182, B-183, B-184, B-185, | |
| B-186, B-187, B-188, B-189, B-190, | |
| B-191, B-192, B-193, B-194, B-195, | |
| B-196, B-197, B-198, B-199, B-200, | |
| B-201, B-202, B-203, B-204, B-205, | |
| B-206, B-207, B-208, B-209, B-210, | |
| B-211, B-212, B-213, B-214, B-215, | |
| B-216, B-217, B-218, B-219, B-220, | |
| B-221, B-222, B-223, B-224, B-225, | |
| B-226, B-227, B-228, B-229, B-230, | |
| B-231, B-232,B233, B-234, B-235, | |
| B-236, B-237, B-238, B-239, B-240, | |
| B-241, B-242, B-243B-244, B-245, | |
| B-246, B-247, B-248, B-249, B-250, | |
| B-251, B-252 | |
| a compound selected from | valaciclovir |
| the group consisting of | |
| B-1, B-2, B-3, B-4, B-5, | |
| B-6, B-7, B-8, B-9, B-10, | |
| B-11, B-12, B-13, B-14, B-15, | |
| B-16, B-17, B-18, B-19, B-20, | |
| B-21, B-22, B-23, B-24, B-25, | |
| B-26, B-27, B-28, B-29, B-30, | |
| B-31, B-32, B-33, B-34, B-35, | |
| B-36, B-37, B-38, B-39, B-40, | |
| B-41, B-42, B-43, B-44, B-45, | |
| B-46, B-47, B-48, B-49, B-50, | |
| B-51, B-52, B-53, B-54, B-55, | |
| B-56, B-57, B-58, B-59, B-60, | |
| B-61, B-62, B-63, B-64, B-65, | |
| B-66, B-67, B-68, B-69, B-70, | |
| B-71, B-72, B-73, B-74, B-75, | |
| B-76, B-77, B-78, B-79, B-80, | |
| B-81, B-82, B-83, B-84, B-85, | |
| B-86, B-87, B-88, B-89, B-90, | |
| B-91, B-92, B-93, B-94, B-95, | |
| B-96, B-97, B-98, B-99, B-100, | |
| B-101, B-102, B-103, B-104, B-105, | |
| B-106, B-107, B-108, B-109, B-110, | |
| B-111, B-112, B-113, B-114, B-115, | |
| B-116, B-117, B-118, B-119, B-120, | |
| B-121, B-122, B-123, B-124, B-125, | |
| B-126, B-127, B-128, B-129, B-130, | |
| B-131, B-132, B-133, B-134, B-135, | |
| B-136, B-137, B-138, B-139, B-140, | |
| B-141, B-142, B-143, B-144, B-145, | |
| B-146, B-147, B-148, B-149, B-150, | |
| B-151, B-152, B-153, B-154, B-155, | |
| B-156, B-157, B-158, B-159, B-160, | |
| B-161, B-162, B-163, B-164, B-165, | |
| B-166, B-167, B-168, B-169, B-170, | |
| B-171, B-172, B-173, B-174, B-175, | |
| B-176, B-177, B-178, B-179, B-180, | |
| B-181, B-182, B-183, B-184, B-185, | |
| B-186, B-187, B-188, B-189, B-190, | |
| B-191, B-192, B-193, B-194, B-195, | |
| B-196, B-197, B-198, B-199, B-200, | |
| B-201, B-202, B-203, B-204, B-205, | |
| B-206, B-207, B-208, B-209, B-210, | |
| B-211, B-212, B-213, B-214, B-215, | |
| B-216, B-217, B-218, B-219, B-220, | |
| B-221, B-222, B-223, B-224, B-225, | |
| B-226, B-227, B-228, B-229, B-230, | |
| B-231, B-232,B233, B-234, B-235, | |
| B-236, B-237, B-238, B-239, B-240, | |
| B-241, B-242, B-243B-244, B-245, | |
| B-246, B-247, B-248, B-249, B-250, | |
| B-251, B-252 | |

By way of yet further example, Table C3 details additional suitable combinations that may be employed in the methods and compositions of the current invention. The combination may also include an isomer, a pharmaceutically acceptable salt, ester, or prodrug of any of the cyclooxygenase-2 selective inhibitors or anti-herpes virus agents listed in Table C3.

TABLE NO. C3

| Cyclooxygenase-2 | Anti-Herpes |
|---|---|
| Selective Inhibitor | Virus Agents |
| celecoxib | ganciclovir |
| celecoxib | foscarnet |
| celecoxib | cidofovir |
| celecoxib | acycloguanosine |
| celecoxib | tri-fluorothymidine |
| celecoxib | acyclovir |
| celecoxib | famciclovir |
| ceracoxib | valaciclovir |
| deracoxib | ganciclovir |
| deracoxib | foscarnet |
| deracoxib | cidofovir |
| deracoxib | acycloguanosine |
| deracoxib | tri-fluorothymidine |
| deracoxib | acyclovir |
| deracoxib | famciclovir |
| deracoxib | valaciclovir |
| valdecoxib | ganciclovir |
| valdecoxib | foscarnet |
| valdecoxib | cidofovir |
| valdecoxib | acycloguanosine |
| valdecoxib | tri-fluorothymidine |
| valdecoxib | acyclovir |
| valdecoxib | famciclovir |
| valdecoxib | valaciclovir |
| rofecoxib | ganciclovir |
| rofecoxib | foscarnet |
| rofecoxib | cidofovir |
| rofecoxib | acycloguanosine |
| rofecoxib | tri-fluorothymidine |
| rofecoxib | acyclovir |
| rofecoxib | famciclovir |
| rofecoxib | valaciclovir |
| etoricoxib | ganciclovir |
| etoricoxib | foscarnet |
| etoricoxib | cidofovir |
| etoricoxib | acycloguanosine |
| etoricoxib | tri-fluorothymidine |
| etoricoxib | acyclovir |
| etoricoxib | famciclovir |
| etoricoxib | valaciclovir |
| meloxicam | ganciclovir |
| meloxicam | foscarnet |
| meloxicam | cidofovir |
| meloxicam | acycloguanosine |
| meloxicam | tri-fluorothymidine |
| meloxicam | acyclovir |
| meloxicam | famciclovir |
| meloxicam | valaciclovir |
| parecoxib | ganciclovir |
| parecoxib | foscarnet |
| parecoxib | cidofovir |
| parecoxib | acycloguanosine |
| parecoxib | tri-fluorothymidine |
| parecoxib | acyclovir |
| parecoxib | famciclovir |
| parecoxib | valaciclovir |
| 4-(4-cyclohexyl-2- | ganciclovir |
| methyloxazol-5-yl)-2- | |
| fluorobenzenesulfonamide | |
| 4-(4-cyclohexyl-2- | foscarnet |
| methyloxazol-5-yl)-2- | |
| fluorobenzenesulfonamide | |
| 4-(4-cyclohexyl-2- | cidofovir |
| methyloxazol-5-yl)-2- | |
| fluorobenzenesulfonamide | |
| 4-(4-cyclohexyl-2- | acycloguanosine |
| methyloxazol-5-yl)-2- | |
| fluorobenzenesulfonamide | |
| 4-(4-cyclohexyl-2- | tri-fluorothymidine |
| methyloxazol-5-yl)-2- | |
| fluorobenzenesulfonamide | |
| 4-(4-cyclohexyl-2- | acyclovir |
| methyloxazol-5-yl)-2- | |
| fluorobenzenesulfonamide | |
| 4-(4-cyclohexyl-2- | famciclovir |
| methyloxazol-5-yl)-2- | |
| fluorobenzenesulfonamide | |
| 4-(4-cyclohexyl-2- | valaciclovir |
| methyloxazol-5-yl)-2- | |
| fluorobenzenesulfonamide | |
| 2-(3,5-difluorophenyl)-3-(4- | ganciclovir |
| (methylsulfonyl)phenyl)-2- | |
| cyclopenten-1-one | |
| 2-(3,5-difluorophenyl)-3-(4- | foscarnet |
| (methylsulfonyl)phenyl)-2- | |
| cyclopenten-1-one | |
| 2-(3,5-difluorophenyl)-3-(4- | cidofovir |
| (methylsulfonyl)phenyl)-2- | |
| cyclopenten-1-one | |
| 2-(3,5-difluorophenyl)-3-(4- | acycloguanosine |
| (methylsulfonyl)phenyl)-2- | |
| cyclopenten-1-one | |
| 2-(3,5-difluorophenyl)-3-(4- | tri-fluorothymidine |
| (methylsulfonyl)phenyl)-2- | |
| cyclopenten-1-one | |
| 2-(3,5-difluorophenyl)-3-(4- | acyclovir |
| (methylsulfonyl)phenyl)-2- | |
| cyclopenten-1-one | |
| 2-(3,5-difluorophenyl)-3-(4- | famciclovir |
| (methylsulfonyl)phenyl)-2- | |
| cyclopenten-1-one | |
| 2-(3,5-difluorophenyl)-3-(4- | valaciclovir |
| (methylsulfonyl)phenyl)-2- | |
| cyclopenten-1-one | |
| N-[2-(cyclohexyloxy)-4- | ganciclovir |
| nitrophenyl]methanesulfonamide | |
| N-[2-(cyclohexyloxy)-4- | foscarnet |
| nitrophenyl]methanesulfonamide | |
| N-[2-(cyclohexyloxy)-4- | cidofovir |
| nitrophenyl]methanesulfonamide | |
| N-[2-(cyclohexyloxy)-4- | acycloguanosine |
| nitrophenyl]methanesulfonamide | |
| N-[2-(cyclohexyloxy)-4- | tri-fluorothymidine |
| nitrophenyl]methanesulfonamide | |
| N-[2-(cyclohexyloxy)-4- | acyclovir |
| nitrophenyl]methanesulfonamide | |
| N-[2-(cyclohexyloxy)-4- | famciclovir |
| nitrophenyl]methanesulfonamide | |
| N-[2-(cyclohexyloxy)-4- | valaciclovir |
| nitrophenyl]methanesulfonamide | |
| 2-(3,4-difluorophenyl)-4-(3- | ganciclovir |
| hydroxy-3-methylbutoxy)-5-[4- | |
| (methylsulfonyl)phenyl]- | |
| 3(2H)-pyridazinone | |
| 2-(3,4-difluorophenyl)-4-(3- | foscarnet |
| hydroxy-3-methylbutoxy)-5-[4- | |
| (methylsulfonyl)phenyl]- | |
| 3(2H)-pyridazinone | |
| 2-(3,4-difluorophenyl)-4-(3- | cidofovir |
| hydroxy-3-methylbutoxy)-5-[4- | |
| (methylsulfonyl)phenyl]- | |
| 3(2H)-pyridazinone | |
| 2-(3,4-difluorophenyl)-4-(3- | acycloguanosine |
| hydroxy-3-methylbutoxy)-5-[4- | |
| (methylsulfonyl)phenyl]- | |
| 3(2H)-pyridazinone | |
| 2-(3,4-difluorophenyl)-4-(3- | tri-fluorothymidine |
| hydroxy-3-methylbutoxy)-5-[4- | |
| (methylsulfonyl)phenyl]- | |
| 3(2H)-pyridazinone | |
| 2-(3,4-difluorophenyl)-4-(3- | acyclovir |
| hydroxy-3-methylbutoxy)-5-[4- | |
| (methylsulfonyl)phenyl]- | |
| 3(2H)-pyridazinone | |
| 2-(3,4-difluorophenyl)-4-(3- | famciclovir |
| hydroxy-3-methylbutoxy)-5-[4- | |
| (methylsulfonyl)phenyl]- | |
| 3(2H)-pyridazinone | |
| 2-(3,4-difluorophenyl)-4-(3- | valaciclovir |
| hydroxy-3-methylbutoxy)-5-[4- | |
| (methylsulfonyl)phenyl]- | |
| 3(2H)-pyridazinone | |
| 2-[(2,4-dichloro-6- | ganciclovir |
| methylphenyl)amino]-5- | |
| ethyl-benzeneacetic acid | |
| 2-[(2,4-dichloro-6- | foscarnet |
| methylphenyl)amino]-5- | |
| ethyl-benzeneacetic acid | |
| 2-[(2,4-dichloro-6- | cidofovir |
| methylphenyl)amino]-5- | |
| ethyl-benzeneacetic acid | |
| 2-[(2,4-dichloro-6- | acycloguanosine |
| methylphenyl)amino]-5- | |
| ethyl-benzeneacetic acid | |
| 2-[(2,4-dichloro-6- | tri-fluorothymidine |
| methylphenyl)amino]-5- | |
| ethyl-benzeneacetic acid | |
| 2-[(2,4-dichloro-6- | acyclovir |
| methylphenyl)amino]-5- | |
| ethyl-benzeneacetic acid | |
| 2-[(2,4-dichloro-6- | famciclovir |
| methylphenyl)amino]-5- | |
| ethyl-benzeneacetic acid | |
| 2-[(2,4-dichloro-6- | valaciclovir |
| methylphenyl)amino]-5- | |
| ethyl-benzeneacetic acid | |
| (3Z)-3-[(4-chlorophenyl) | ganciclovir |
| [4-(methylsulfonyl)phenyl] | |
| methylene]dihydro-2(3H)- | |
| furanone | |
| (3Z)-3-[(4-chlorophenyl) | foscarnet |
| [4-(methylsulfonyl)phenyl] | |
| methylene]dihydro-2(3H)- | |
| furanone | |
| (3Z)-3-[(4-chlorophenyl) | cidofovir |
| [4-(methylsulfonyl)phenyl] | |
| methylene]dihydro-2(3H)- | |
| furanone | |
| (3Z)-3-[(4-chlorophenyl) | acycloguanosine |
| [4-(methylsulfonyl)phenyl] | |
| methylene]dihydro-2(3H)- | |
| furanone | |
| (3Z)-3-[(4-chlorophenyl) | tri-fluorothymidine |
| [4-(methylsulfonyl)phenyl] | |
| methylene]dihydro-2(3H)- | |
| furanone | |
| (3Z)-3-[(4-chlorophenyl) | acyclovir |
| [4-(methylsulfonyl)phenyl] | |
| methylene]dihydro-2(3H)- | |
| furanone | |
| (3Z)-3-[(4-chlorophenyl) | famciclovir |
| [4-(methylsulfonyl)phenyl] | |
| methylene]dihydro-2(3H)- | |
| furanone | |
| (3Z)-3-[(4-chlorophenyl) | valaciclovir |
| [4-(methylsulfonyl)phenyl] | |
| methylene]dihydro-2(3H)- | |
| furanone | |
| (S)-6,8-dichloro-2- | ganciclovir |
| (trifluoromethyl)-2H-1- | |
| benzopyran-3-carboxylic | |
| acid | |
| (S)-6,8-dichloro-2- | foscarnet |
| (trifluoromethyl)-2H-1- | |
| benzopyran-3-carboxylic | |
| acid | |
| (S)-6,8-dichloro-2- | cidofovir |
| (trifluoromethyl)-2H-1- | |
| benzopyran-3-carboxylic | |
| acid | |
| (S)-6,8-dichloro-2- | acycloguanosine |
| (trifluoromethyl)-2H-1- | |
| benzopyran-3-carboxylic | |
| acid | |
| (S)-6,8-dichloro-2- | tri-fluorothymidine |
| (trifluoromethyl)-2H-1- | |
| benzopyran-3-carboxylic | |
| acid | |
| (S)-6,8-dichloro-2- | acyclovir |
| (trifluoromethyl)-2H-1- | |
| benzopyran-3-carboxylic | |
| acid | |
| (S)-6,8-dichloro-2- | famciclovir |
| (trifluoromethyl)-2H-1- | |
| benzopyran-3-carboxylic | |
| acid | |
| (S)-6,8-dichloro-2- | valaciclovir |
| (trifluoromethyl)-2H-1- | |
| benzopyran-3-carboxylic | |
| acid | |
| lumiracoxib | ganciclovir |
| lumiracoxib | foscarnet |
| lumiracoxib | cidofovir |
| lumiracoxib | acycloguanosine |
| lumiracoxib | tri-fluorothymidine |
| lumiracoxib | acyclovir |
| lumiracoxib | famciclovir |
| lumiracoxib | valaciclovir |

Diagnosis of a Herpes Virus Infection

One aspect of the invention encompasses diagnosing a subject in need of treatment or prevention for a herpes virus infection. A number of suitable methods for diagnosing a herpes virus infection in a subject may be used in the practice of the invention. Generally speaking, the type of test employed for diagnosis is dependent upon the subject's physical symptoms. In one embodiment, the subject will typically have a visible outbreak and will see a physician while symptoms are still present. The physician will visually examine the area, take a sample from the lesions and then have the sample tested to see if the herpes virus is present. The test utilized for diagnosis is typically a specific virus culture or assay for herpes virus. By way of example, suitable tests include, cell culture tests, antigen tests, or pap smear tests.

In another embodiment, a subject has concerns about herpes, but does not have any visible symptoms. Typically, when no visible symptoms are present, a blood test is utilized for diagnosis. In one alternative of this embodiment, a Western blot designed to detect antibodies against herpes virus present in a subject's blood is employed for diagnosis. Other suitable tests to diagnose a herpes infection from a blood sample include the Meridian Diagnostics test for HSV-1 and Diagnology's POCkit® HSV-2 Rapid Test for HSV-2. In addition to the methods identified herein, a number of other suitable methods known in the art for diagnosis of a herpes virus infection may be utilized.

Indication to be Treated

Generally speaking, the composition comprising a cyclooxygenase-2 selective inhibitor or a combination of a cyclooxygenase-2 selective inhibitor and an anti-herpes virus agent may be used to treat or prevent herpes virus infection in a subject during each stage of disease progression including first-episode primary infection, first-episode nonprimary infection, and recurrent infections. Typically when the subject is a human, the composition is employed to treat or prevent herpes simplex virus type 1 (HSV-1), herpes simplex virus type 2 (HSV-2), cytomegalovirus (CMV), and varicella zoster virus (VZV) infections. Even more typically when the subject is a human, the composition is utilized to treat or prevent either a HSV-1 or a HSV-2 infection.

One aspect of the invention embraces a method for the treatment of a subject who has a first-episode primary infection. First-episode primary infection occurs when an individual is antibody-negative to either HSV-1 or HSV-2. This infection is characterized by localized and/or systemic symptoms that sometimes require hospitalization. Local symptoms consist of multiple, small, painful, vesicular lesions on the external genitalia that tend to be more severe in women. Lesions may appear in clusters that coalesce into larger areas of ulceration. After 2-4 weeks, the sores either crust over or resolve and the virus enters dorsal sacral nerve roots, where it remains in a latent stage until it becomes reactivated. Other local symptoms may include itching, dysuria, vaginal discharge, and tender inguinal adenopathy. Systemic manifestations include fever, headache, malaise and myalgia that usually resolve after a week.

A further aspect of the invention encompasses a method for the treatment of a subject who has a first-episode nonprimary infection. A first-episode nonprimary infection occurs in persons with preexisting antibodies, usually from previous oral HSV-1 infection. Nonprimary episodes produce symptoms that are often milder and with fewer lesions that are less painful and shorter in duration, commonly only lasting a week.

Yet another aspect of the invention provides a method to treat or prevent recurrent episodes by administrating the composition as a part of a suppressive therapy regime. Suppressive treatment involves taking the composition on a daily basis for the lifetime of the subject to reduce the chance of an episode occurring. Recurrent infections typically produce milder symptoms localized to the genital area and depending upon the particular subject, may occur from 1 to several times per year for the lifetime of the subject. Subjects may also experience prodromal symptoms consisting of tingling or itching a few hours to two days before lesions appear, inguinal swelling, pain, fever, malaise, headaches, muscle aches, and swollen glands. Typically, only subjects having either frequent (e.g. about 3 or more on a yearly basis) or particularly severe recurrent infections participate in a suppressive treatment regime.

The invention also embraces the treatment of either subjects who are at risk or have an abnormally elevated risk of being infected with a herpes virus. The subject may have on-going sexual relations with an individual known to be infected with HSV-1 or HSV-2. Moreover, the subject may have a depressed immune response, such as a subject with acquired immunodeficiency syndrome or a subject with an autoimmune disease. The subject may also be one that engages in risky and/or frequent sexual encounters.

A further aspect of the invention provides compositions to treat herpes related disorders. Herpes related disorders include a number of different symptoms associated with or resulting from a herpes infection. By way of example, when the herpes infection results either in a first-episode primary infection or a first-episode nonprimary infection, the related disorders often include pain, fever, headache, malaise and myalgia. In addition to the cyclooxygenase-2 selective inhibitor and anti-herpes viral agent, the composition of the invention may also include another agent that effectively attenuates the particular related disorder. In one embodiment, the additional agent may include an anti-inflammatory agent that is not a cyclooxygenase-2 selective inhibitor. In one alternative of this embodiment, the anti-inflammatory agent is a non-steroidal anti-inflammatory agent. Suitable non-steroidal anti-inflammatory agents include naproxen sodium, diclofenac, suilindace, oxaprozin, diflunisal, aspirin, piroxicam, indomethocin, etodolac, ibuprofen, fenoprofen, ketoprofen, mefenamic acid, nabumetone, tolmetin sodium, and ketorolac trometharnine. In an alternative of this embodiment, the non-steroidal anti-inflammatory agent is acetaminophen. In another alternative of this embodiment, the anti-inflammatory agent is a steroid.

### EXAMPLES

### Example 1

### Evaluation of COX-1 and COX-2 Activity In Vitro

The COX-2 inhibitors suitable for use in this invention exhibit selective inhibition of COX-2 over COX-1 when tested in vitro according to the following activity assays.

Preparation of Recombinant COX Baculoviruses

Recombinant COX-1 and COX-2 are prepared as described by Gierse et al, [J. Biochem., 305, 479-84 (1995)]. A 2.0 kb fragment containing the coding region of either human or murine COX-1 or human or murine COX-2 is cloned into a Bam-H1 site of the baculovirus transfer vector pVL1393 (Invitrogen) to generate the baculovirus transfer vectors for COX-1 and COX-2 in a manner similar to the method of D. R. O'Reilly et al (Baculovirus Expression Vectors: A Laboratory Manual (1992)). Recombinant baculoviruses are isolated by transfecting 4 μg of baculovirus transfer vector DNA into SF9 insect cells (2×10⁸) along with 200 ng of linearized baculovirus plasmid DNA by the calcium phosphate method. See M. D. Summers and G. E. Smith, A Manual of Methods for Baculovirus Vectors and Insect Cell Culture Procedures, Texas Agric. Exp. Station Bull. 1555 (1987). Recombinant viruses are purified by three rounds of plaque purification and high titer (10⁷-10⁸ pfu/mL) stocks of virus are prepared. For large scale production, SF9 insect cells are infected in 10 liter fermentors (0.5×106/mL) with the recombinant baculovirus stock such that the multiplicity of infection is 0.1. After 72 hours the cells are centrifuged and the cell pellet is homogenized in Tris/Sucrose (50 mM: 25%, pH 8.0) containing 1% 3-[(3-cholamidopropyl)-dimethylammonio]-1-propanesulfonate (CHAPS). The homogenate is centrifuged at 10,000×G for 30 minutes, and the resultant supernatant is stored at −80° C. before being assayed for COX activity.

Assay for COX-1 and COX-2 Activity

COX activity is assayed as PGE2 formed/μg protein/time using an ELISA to detect the prostaglandin released. CHAPS-solubilized insect cell membranes containing the appropriate COX enzyme are incubated in a potassium phosphate buffer (50 mM, pH 8.0) containing epinephrine, phenol, and heme with the addition of arachidonic acid (10 μM). Compounds are pre-incubated with the enzyme for 10-20 minutes prior to the addition of arachidonic acid. Any reaction between the arachidonic acid and the enzyme is stopped after ten minutes at 37° C. by transferring 40 μl of reaction mix into 160 μl ELISA buffer and 25 μM indomethacin. The PGE2 formed is measured by standard ELISA technology (Cayman Chemical).

Fast Assay for COX-1 and COX-2 Activity

COX activity is assayed as PGE2 formed/μg protein/time using an ELISA to detect the prostaglandin released. CHAPS-solubilized insect cell membranes containing the appropriate COX enzyme are incubated in a potassium phosphate buffer (0.05 M Potassium phosphate, pH 7.5, 2 μM phenol, 1 μM heme, 300 μM epinephrine) with the addition of 20 μl of 100 μM arachidonic acid (10 μM). Compounds are pre-incubated with the enzyme for 10 minutes at 25° C. prior to the addition of arachidonic acid. Any reaction between the arachidonic acid and the enzyme is stopped after two minutes at 37° C. by transferring 40 μl of reaction mix into 160 μl ELISA buffer and 25 μM indomethacin. Indomethacin, a non-selective COX-2/COX-1 inhibitor, may be utilized as a positive control. The PGE₂ formed is typically measured by standard ELISA technology utilizing a PGE2 specific antibody, available from a number of commercial sources.

Each compound to be tested may be individually dissolved in 2 ml of dimethyl sulfoxide (DMSO) for bioassay testing to determine the COX-1 and COX-2 inhibitory effects of each particular compound. Potency is typically expressed by the IC₅₀ value expressed as g compound/ml solvent resulting in a 50% inhibition of PGE2 production. Selective inhibition of COX-2 may be determined by the IC₅₀ ratio of COX-1/COX-2.

By way of example, a primary screen may be performed in order to determine particular compounds that inhibit COX-2 at a concentration of 10 ug/ml. The compound may then be subjected to a confirmation assay to determine the extent of COX-2 inhibition at three different concentrations (e.g., 10 ug/ml, 3.3 ug/ml and 1.1 ug/ml). After this screen, compounds can then be tested for their ability to inhibit COX-1 at a concentration of 10 ug/ml. With this assay, the percentage of COX inhibition compared to control can be determined, with a higher percentage indicating a greater degree of COX inhibition. In addition, the IC₅₀ value for COX-1 and COX-2 can also be determined for the tested compound. The selectivity for each compound may then be determined by the IC₅₀ ratio of COX-1/COX-2, as set-forth above.

### Example 2

### In Vitro and In Vivo Testing of Combination and Mono Therapies of the Invention

In the examples below, a combination therapy contains an antiviral agent and a Cox-2 selective inhibitor and a mono therapy contains a Cox-2 selective inhibitor alone. The efficacy of such combination or mono therapy can be evaluated in comparison to a control treatment such as a placebo treatment. By way of example, a combination therapy can contain acyclovir and valdecoxib, famciclovir and valdecoxib, or 5-fluorodeoxyuridine and rofecoxib. And, a mono therapy may include valdecoxib, celecoxib or rofecoxib. It should be noted that these are only several examples, and that any of the antiviral agents and Cox-2 inhibitors detailed herein, such as the combinations detailed in Tables C1, C2, or C3, may be tested as a combination therapy and any of the Cox-2 selective inhibitors detailed herein may be tested as a mono therapy. The dosages to be employed in a combination therapy or mono therapy may be readily determined by a skilled artisan conducting the study. The length of the study treatment will vary on a particular study and can also be determined by one of ordinary skill in the art. By way of example, the combination or mono therapy may be administered for 3-4 weeks. The antiviral agent and Cox-2 inhibitor can be administered by any route as described herein, but are preferably administered orally for human subjects.

In Vitro Assays

In vitro studies can be conducted as described in Sainz and Halford, Journal of Virology, Vol. 76, No. 22, pp.11541-11550, 2002.

Vero cells are propagated in Dulbecco modified Eagle medium (DMEM) containing 0.15% HCO₃-supplemented with 10% fetal bovine serum, penicillin G (100 U/ml), and streptomycin (100 mg/ml), hereafter referred to as complete DMEM. Wild-type HSV-1 strain KOS is propagated in Vero cells.

Plaque Reduction and Virus Replication Assays

For plaque reduction assays, Vero cells can be seeded in 12-well plates at a density of 9×10⁴ cells per well, and 6 h later a combination therapy or mono therapy is added to the culture medium. Vero cells are inoculated with HSV-1 12 h later, and 1 h later the medium is replaced with complete DMEM containing 0.5% methylcellulose and the same combination therapy or mono therapy used in the pretreatment. As a control, the same protocol is repeated with cells that receive a vehicle in which the combination therapy or mono therapy is administered. Plaques are counted 2 to 3 days later.

Replication Assay

For viral replication assays, Vero cells are seeded in 24-well plates at a density of 5×10⁴ cells per well, and 6 h later the cultures are each treated with vehicle, or several different dosages of the combination therapy or mono therapy. Cells are inoculated 12 h later with HSV-1 at the indicated multiplicity of infection (MOI), e.g., 0.1 to 20 PFU/cell. One hour later cells are rinsed twice with 0.5 ml of complete DMEM, and the well is treated with complete DMEM containing the same combination therapy or mono therapy present during the pretreatment. The cultures are freeze-thawed 24 h after infection, and the viral titer is determined on Vero cells by a 96-well microtiter plaque assay.

PCR Analysis of HSV-1-Infected Vero cells

Vero cells are plated in 24-well plates, and 6 h later the cells are treated with vehicle or the combination therapy or the mono therapy (several different dosages). Cells are inoculated 12 h later with HSV-1 strain KOS at an MOI of 0.1 to 20 PFU per cell. One hour later, cells are washed twice with 0.5 ml of complete DMEM, and DNA is isolated from each culture by a phenol-chloroform extraction procedure (60). PCR is performed on DNA samples to amplify a 243-bp fragment of the HSV-1 ribonucleotide reductase (RR) gene by using the oligonucleotide provided in the paper. The yield of 243-bp PCR product amplified from the DNA samples is quantified by densitometric analysis of ethidium bromide-stained agarose gels (Alpha Inotech Corp., San Leandro, Calif.).

Expected Results

The efficiency of HSV-1 strain KOS plaque formation is compared in Vero cell monolayers treated with a vehicle, combination treatment, and mono therapy. It is expected that the Plaque formation will be reduced on Vero cell monolayers treated with the combination therapy or monotherapy as compared to the vehicle treatment. It is also expected that HSV-1 replication will be reduced in combination therapy or mono therapy treated cultures relative to vehicle-treated cultures.

PCR assay can be used to compare the relative efficiency with which HSV-1 adsorbs to Vero cells treated with (1) vehicle ; (2) combination treatment; or (3) mono therapy. One hour after inoculations with MOIs of 0.1 to 20 PFU/cell, DNA is isolated from HSV-1-infected Vero cells, and PCR is used to amplify a 243-bp fragment of the HSV-1 genome. The HSV-1 RR PCR product yield increases as a function of viral MOI in Vero cells treated with vehicle, mono therapy or the combination treatment. Thus, PCR provides a valid basis for comparing the relative amount of HSV-1 DNA that enter Vero cells.

In Vivo Assays

The laboratory animal study can generally be performed as described in Peng et al., Journal of Virology, Vol. 72, No. 1, pp.65-72, 1998.

Murine Flank (Zosteriform) Model of HSV Challenge

A zosteriform model of HSV-1 infection can be used to test the efficacy of the combination therapy and mono therapy. Nine- to ten-week-old BALB/c mice can be sued in these experiments. The right flank of each test or control animal is shaved and denuded by using a depilatory cream. Twenty-four hours later, 5×10⁵ PFU of HSV-1 is applied to the depilated flank approximately 3 mm ventral to the spinal column, and the skin is scratched with a 27-gauge needle, using 20 horizontal strokes and 20 vertical strokes over an approximate area of 3 by 3 mm. Following the virus application, BALB/c mice are immunized intraperitoneally with the combination therapy or mono therapy, followed by several additional doses given during the infection, e.g., once a day for at least 10 days. Sham-immunized control animals receive the vehicle at the same intervals, whereas several different doses are given to test mice. The flank is observed daily for at least 10 days, and cumulative scores for primary and secondary areas are recorded from days 3 through 8. The period of recording lesions is generally limited to this period due to the deaths of unprotected animals beginning at day 8. Disease at the inoculation site is scored as follows: 0 points for no disease, 0.5 point for swelling without vesicles, and 1 point each for each vesicle or scab to a maximum score of 5. Swelling and lesions in locations separate from the inoculation site are considered to be secondary or zosteriform disease. Scoring of these lesions is the same as for the inoculation site except that a daily maximal score of 10 is used.

Expected Results

The efficacy of the combination therapy or the mono therapy described herein in treatment of the herpes infection can be established by comparing the degree of the disease exhibited by treated mice and control mice. It is expected that the severity of the disease will be less in mice treated with the combination therapy or the mono therapy.

What is claimed:

1. A method of treating a herpes simplex virus infection, the method comprising:
(a) diagnosing a subject in need of treatment for a herpes simplex virus infection; and
(b) administering to the subject a combination comprising a cyclooxygenase-2 selective inhibitor or an isomer, a pharmaceutically acceptable salt, ester, or prodrug thereof and an anti-herpes simplex virus agent or an isomer, a pharmaceutically acceptable salt, ester, or prodrug thereof, wherein the anti-herpes simplex virus agent is not a cyclooxygenase-2 selective inhibitor.

2. The method of claim 1 wherein the cyclooxgenase-2 selective inhibitor has a selectivity ratio of COX-1 IC₅₀ to COX-2 IC₅₀ not less than about 50.

3. The method of claim 1 wherein the cyclooxgenase-2 selective inhibitor has a selectivity ratio of COX-1 IC₅₀ to COX-2 IC₅₀ not less than about 100.

4. The method of claim 1 wherein the cyclooxygenase-2 selective inhibitor is selected from the group consisting of celecoxib, deracoxib, valdecoxib, rofecoxib, lumiracoxib, etoricoxib, meloxicam, parecoxib, 4-(4-cyclohexyl-2-methyloxazol-5-yl)-2-fluorobenzenesulfonamide, 2-(3,5-difluorophenyl)-3-(4-(methylsulfonyl)phenyl)-2-cyclopenten-1-one, N-[2-(cyclohexyloxy)-4-nitrophenyl]methanesulfonamide, 2-(3,4-difluorophenyl)-4-(3-hydroxy-3-methylbutoxy)-5-[4-(methylsulfonyl)phenyl]-3(2H)-pyridazinone, 2-[(2,4-dichloro-6-methylphenyl)amino]-5-ethyl-benzeneacetic acid, (3Z)-3-[(4-chlorophenyl)[4-(methylsulfonyl)phenyl]methylene]dihydro-2(3H)-furanone, and (S)-6,8-dichloro-2-(trifluoromethyl)-2H-1-benzopyran-3-carboxylic acid.

5. The method of claim 1 wherein the anti-herpes simplex virus agent is selected from the group consisting of ganciclovir, foscarnet, cidofovir, acycloguanosine, tri-fluorothymidine, acyclovir, famciclovir, and valaciclovir.

6. The method of claim 4 wherein the anti-herpes simplex virus agent is selected from the group consisting of ganciclovir, foscarnet, cidofovir, acycloguanosine, tri-fluorothymidine, acyclovir, famciclovir, and valaciclovir.

7. The method of claim 1 wherein the herpes simplex virus is herpes simplex virus-1.

8. The method of claim 1 wherein the herpes simplex virus is herpes simplex virus-2.

9. A method of treating a herpes virus infection, the method comprising:
(a) diagnosing a subject in need of treatment for a herpes virus infection; and
(b) administering to the subject a combination comprising an anti-herpes virus agent or an isomer, a pharmaceutically acceptable salt, ester, or prodrug thereof, wherein the anti-herpes virus agent is not a cyclooxygenase-2 selective inhibitor; and
a cyclooxygenase-2 selective inhibitor or an isomer, a pharmaceutically acceptable salt, ester, or prodrug thereof, wherein the cyclooxygenase-2 selective inhibitor is a chromene compound, the chromene compound comprising a benzothiopyran, a dihydroquinoline or a dihydronaphthalene.

10. The method of claim 9 wherein the cyclooxgenase-2 selective inhibitor has a selectivity ratio of COX-1 IC₅₀ to COX-2 IC₅₀ not less than about 50.

11. The method of claim 9 wherein the cyclooxgenase-2 selective inhibitor has a selectivity ratio of COX-1 IC₅₀ to COX-2 IC₅₀ not less than about 100.

12. The method of claim 9 wherein the cyclooxygenase-2 selective inhibitor is a compound having the formula 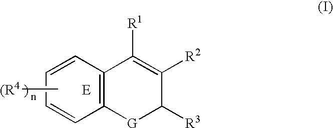
wherein:
n is an integer which is 0, 1, 2, 3 or 4;
G is O, S or NR^{a};
R^{a} is alkyl;
R¹ is selected from the group consisting of H and aryl;
R² is selected from the group consisting of carboxyl, aminocarbonyl, alkylsulfonylaminocarbonyl and alkoxycarbonyl;
R³ is selected from the group consisting of haloalkyl, alkyl, aralkyl, cycloalkyl and aryl optionally substituted with one or more radicals selected from alkylthio, nitro and alkylsulfonyl;
each R⁴ is independently selected from the group consisting of H, halo, alkyl, aralkyl, alkoxy, aryloxy, heteroaryloxy, aralkyloxy, heteroaralkyloxy, haloalkyl, haloalkoxy, alkylamino, arylamino, aralkylamino, heteroarylamino, heteroarylalkylamino, nitro, amino, aminosulfonyl, alkylaminosulfonyl, arylaminosulfonyl, heteroarylaminosulfonyl, aralkylaminosulfonyl, heteroaralkylaminosulfonyl, heterocyclosulfonyl, alkylsulfonyl, hydroxyarylcarbonyl, nitroaryl, optionally substituted aryl, optionally substituted heteroaryl, aralkylcarbonyl, heteroarylcarbonyl, arylcarbonyl, aminocarbonyl, and alkylcarbonyl; and
R⁴ together with the carbon atoms to which it is attached and the remainder of ring E forms a naphthyl radical.

13. The method of claim 12 wherein:
n is an integer which is 0, 1, 2, 3 or 4;
G is O, S or NR^{b};
R¹ is H;
R^{b} is alkyl;
R² is selected from the group consisting of carboxyl, aminocarbonyl, alkylsulfonylaminocarbonyl and alkoxycarbonyl;
R³ is selected from the group consisting of haloalkyl, alkyl, aralkyl, cycloalkyl and aryl, wherein haloalkyl, alkyl, aralkyl, cycloalkyl, and aryl each is independently optionally substituted with one or more radicals selected from the group consisting of alkylthio, nitro and alkylsulfonyl; and
each R⁴ is independently selected from the group consisting of hydrido, halo, alkyl, aralkyl, alkoxy, aryloxy, heteroaryloxy, aralkyloxy, heteroaralkyloxy, haloalkyl, haloalkoxy, alkylamino, arylamino, aralkylamino, heteroarylamino, heteroarylalkylamino, nitro, amino, aminosulfonyl, alkylaminosulfonyl, arylaminosulfonyl, heteroarylaminosulfonyl, aralkylaminosulfonyl, heteroaralkylaminosulfonyl, heterocyclosulfonyl, alkylsulfonyl, optionally substituted aryl, optionally substituted heteroaryl, aralkylcarbonyl, heteroarylcarbonyl, arylcarbonyl, aminocarbonyl, and alkylcarbonyl; or wherein R⁴ together with ring E forms a naphthyl radical.

14. The method of claim 12 wherein:
n is an integer which is 0, 1, 2, 3 or 4;
G is oxygen or sulfur;
R¹ is H;
R² is carboxyl, lower alkyl, lower aralkyl or lower alkoxycarbonyl;
R³ is lower haloalkyl, lower cycloalkyl or phenyl; and
each R⁴ is H, halo, lower alkyl, lower alkoxy, lower haloalkyl, lower haloalkoxy, lower alkylamino, nitro, amino, aminosulfonyl, lower alkylaminosulfonyl, 5-membered heteroarylalkylaminosulfonyl, 6-membered heteroarylalkylaminosulfonyl, lower aralkylaminosulfonyl, 5-membered nitrogen-containing heterocyclosulfonyl, 6-membered-nitrogen containing heterocyclosulfonyl, lower alkylsulfonyl, optionally substituted phenyl, lower aralkylcarbonyl, or lower alkylcarbonyl; or
R⁴ together with the carbon atoms to which it is attached and the remainder of ring E forms a naphthyl radical.

15. The method of claim 12 wherein:
R² is carboxyl;
R³ is lower haloalkyl; and
each R⁴ is H, halo, lower alkyl, lower haloalkyl, lower haloalkoxy, lower alkylamino, amino, aminosulfonyl, lower alkylaminosulfonyl, 5-membered heteroarylalkylaminosulfonyl, 6-membered heteroarylalkylaminosulfonyl, lower aralkylaminosulfonyl, lower alkylsulfonyl, 6-membered nitrogen-containing heterocyclosulfonyl, optionally substituted phenyl, lower aralkylcarbonyl, or lower alkylcarbonyl; or
R⁴ together with ring E forms a naphthyl radical.

16. The method of claim 9 wherein the cyclooxgyenase-2 selective inhibitor is (S)-6,8-dichloro-2-(trifluoromethyl)-2H-1-benzopyran-3-carboxylic acid.

17. The method of claim 9 wherein the anti-herpes virus agent is selected from the group consisting of ganciclovir, foscamet, cidofovir, acycloguanosine, tri-fluorothymidine, acyclovir, famciclovir, and valaciclovir.

18. The method of claim 9 wherein the herpes virus is herpes simplex virus-1 or herpes simplex virus-2.

19. The method of claim 9 wherein the herpes virus is cytomegalovirus.

20. The method of claim 9 wherein the herpes virus is varicella zoster virus.

21. A method of treating a herpes virus infection, the method comprising:
(a) diagnosing a subject in need of treatment for a herpes virus infection; and
(b) administering to the subject a combination comprising an anti-herpes virus agent or an isomer, a pharmaceutically acceptable salt, ester, or prodrug thereof, wherein the anti-herpes virus agent is not a cyclooxygenase-2 selective inhibitor; and
a cyclooxygenase-2 selective inhibitor or an isomer, a pharmaceutically acceptable salt, ester, or prodrug thereof, wherein the cyclooxygenase-2 selective inhibitor is a tricyclic compound, the tricyclic compound comprising a benzenesulfonamide or methylsulfonylbenzene.

22. The method of claim 21 wherein the cyclooxgenase-2 selective inhibitor has a selectivity ratio of COX-1 IC₅₀ to COX-2 IC₅₀ not less than about 50.

23. The method of claim 21 wherein the cyclooxgenase-2 selective inhibitor has a selectivity ratio of COX-1 IC₅₀ to COX-2 IC₅₀ not less than about 100.

24. The method of claim 21 wherein the cyclooxygenase-2 selective inhibitor is a compound of the formula: 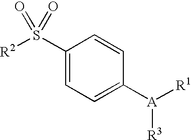
wherein:
A is selected from the group consisting of partially unsaturated or unsaturated heterocyclyl and partially unsaturated or unsaturated carbocyclic rings;
R¹ is selected from the group consisting of heterocyclyl, cycloalkyl, cycloalkenyl and aryl, wherein R¹ is optionally substituted at a substitutable position with one or more radicals selected from alkyl, haloalkyl, cyano, carboxyl, alkoxycarbonyl, hydroxyl, hydroxyalkyl, haloalkoxy, amino, alkylamino, arylamino, nitro, alkoxyalkyl, alkylsulfinyl, halo, alkoxy and alkylthio;
R² is selected from the group consisting of methyl or amino; and
R³ is selected from the group consisting of a radical selected from H, halo, alkyl, alkenyl, alkynyl, oxo, cyano, carboxyl, cyanoalkyl, heterocyclyloxy, alkyloxy, alkylthio, alkylcarbonyl, cycloalkyl, aryl, haloalkyl, heterocyclyl, cycloalkenyl, aralkyl, heterocyclylalkyl, acyl, alkylthioalkyl, hydroxyalkyl, alkoxycarbonyl, arylcarbonyl, aralkylcarbonyl, aralkenyl, alkoxyalkyl, arylthioalkyl, aryloxyalkyl, aralkylthioalkyl, aralkoxyalkyl, alkoxyaralkoxyalkyl, alkoxycarbonylalkyl, aminocarbonyl, aminocarbonylalkyl, alkylaminocarbonyl, N-arylaminocarbonyl, N-alkyl-N-arylaminocarbonyl, alkylaminocarbonylalkyl, carboxyalkyl, alkylamino, N-arylamino, N-aralkylamino, N-alkyl-N-aralkylamino, N-alkyl-N-arylamino, aminoalkyl, alkylaminoalkyl, N-arylaminoalkyl, N-aralkylaminoalkyl, N-alkyl-N-aralkylaminoalkyl, N-alkyl-N-arylaminoalkyl, aryloxy, aralkoxy, arylthio, aralkylthio, alkylsulfinyl, alkylsulfonyl, aminosulfonyl, alkylaminosulfonyl, N-arylaminosulfonyl, arylsulfonyl, N-alkyl-N-arylaminosulfonyl.

25. The method of claim 21 wherein the cyclooxygenase-2 selective inhibitor is selected from the group consisting of celecoxib, valdecoxib, parecoxib, deracoxib, rofecoxib, etoricoxib, and 2-(3,4-difluorophenyl)-4-(3-hydroxy-3-methylbutoxy)-5-[4-(methylsulfonyl)phenyl]-3(2H)-pyridazinone.

26. The method of claim 21 wherein the anti-herpes virus agent is selected from the group consisting of ganciclovir, foscarnet, cidofovir, acycloguanosine, tri-fluorothymidine, acyclovir, famciclovir, and valaciclovir.

27. The method of claim 21 wherein the herpes virus is herpes simplex virus-1 or herpes simplex virus-2.

28. The method of claim 21 wherein the herpes virus is cytomegalovirus.

29. The method of claim 21 wherein the herpes virus is varicella zoster virus.

30. A method of treating a herpes virus infection, the method comprising:
(a) diagnosing a subject in need of treatment for a herpes virus infection; and
(b) administering to the subject a combination comprising an anti-herpes virus agent or an isomer, a pharmaceutically acceptable salt, ester, or prodrug thereof, wherein the anti-herpes virus agent is not a cyclooxygenase-2 selective inhibitor; and
a cyclooxygenase-2 selective inhibitor or an isomer, a pharmaceutically acceptable salt, ester, or prodrug thereof, wherein the cyclooxygenase-2 selective inhibitor is a phenyl acetic acid compound.

31. The method of claim 30 wherein the cyclooxgenase-2 selective inhibitor has a selectivity ratio of COX-1 IC₅₀ to COX-2 IC₅₀ not less than about 50.

32. The method of claim 30 wherein the cyclooxgenase-2 selective inhibitor has a selectivity ratio of COX-1 IC₅₀ to COX-2 IC₅₀ not less than about 100.

33. The method of claim 30 wherein the cyclooxygenase-2 selective inhibitor is a compound having the formula: 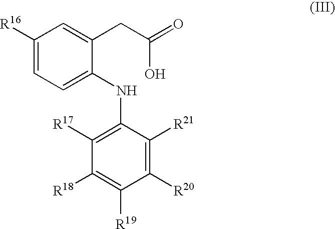
wherein:
R¹⁶ is methyl or ethyl;
R¹⁷ is chloro or fluoro;
R¹⁸ is hydrogen or fluoro;
R¹⁹ is hydrogen, fluoro, chloro, methyl, ethyl, methoxy, ethoxy or hydroxy;
R²⁰ is hydrogen or fluoro;
R²¹ is chloro, fluoro, trifluoromethyl or methyl; and
provided that R¹⁷, R¹⁸, R¹⁹ and R²⁰ are not all fluoro when R¹⁶ is ethyl and R¹⁹ is H.

34. The method of claim 33 wherein:
R¹⁶ is ethyl;
R¹⁷ and R¹⁹ are chloro;
R¹⁸ and R²⁰ are hydrogen; and
and R²¹ is methyl.

35. The method of claim 30 wherein the anti-herpes virus agent is selected from the group consisting of ganciclovir, foscarnet, cidofovir, acycloguanosine, tri-fluorothymidine, acyclovir, famciclovir, and valaciclovir.

36. The method of claim 30 wherein the herpes virus is herpes simplex virus-1 or herpes simplex virus-2.

37. The method of claim 30 wherein the herpes virus is cytomegalovirus.

38. The method of claim 30 wherein the herpes virus is varicella zoster virus.

39. A method of treating a herpes virus infection, the method comprising:
(a) diagnosing a subject in need of treatment for a herpes virus infection; and
(b) administering to the subject a combination comprising a cyclooxygenase-2 selective inhibitor selected from the group consisting of celecoxib, deracoxib, valdecoxib, rofecoxib, lumiracoxib, etoricoxib, parecoxib, 2-(3,4-difluorophenyl)-4-(3-hydroxy-3-methylbutoxy)-5-[4-(methylsulfonyl)phenyl]-3(2H)-pyridazinone, and (S)-6,8-dichloro-2-(trifluoromethyl)-2H-1-benzopyran-3-carboxylic acid; and
an anti-herpes virus agent selected from the group consisting of ganciclovir, foscarnet, cidofovir, acycloguanosine, tri-fluorothymidine, acyclovir, famciclovir, and valaciclovir.

40. The method of claim 39 wherein the cyclooxygenase-2 selective inhibitor is celecoxib.

41. The method of claim 39 wherein the cyclooxygenase-2 selective inhibitor is deracoxib.

42. The method of claim 39 wherein the cyclooxygenase-2 selective inhibitor is valdecoxib.

43. The method of claim 39 wherein the cyclooxygenase-2 selective inhibitor is rofecoxib.

44. The method of claim 39 wherein the cyclooxygenase-2 selective inhibitor is etoricoxib.

45. The method of claim 39 wherein the cyclooxygenase-2 selective inhibitor is parecoxib.

46. The method of claim 39 wherein the cyclooxygenase-2 selective inhibitor is 2-(3,4-difluorophenyl)-4-(3-hydroxy-3-methylbutoxy)-5-[4-(methylsulfonyl)phenyl]-3(2H)-pyridazinone.

47. The method of claim 39 wherein the cyclooxygenase-2 selective inhibitor is (S)-6,8-dichloro-2-(trifluoromethyl)-2H-1-benzopyran-3-carboxylic acid.

48. The method of claim 39 wherein the cyclooxygenase-2 selective inhibitor is lumiracoxib.

49. The method of claim 39 wherein the herpes virus is herpes simplex virus-I or herpes simplex virus-2.

50. The method of claim 39 wherein the herpes virus is cytomegalovirus.

51. The method of claim 39 wherein the herpes virus is varicella zoster virus.

52. A composition comprising:
(a) a cyclooxygenase-2 selective inhibitor or an isomer, a pharmaceutically acceptable salt, ester, or prodrug thereof, the inhibitor having the formula: 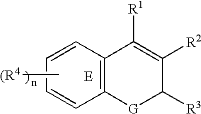
wherein:
n is an integer which is 0, 1, 2, 3 or 4;
G is O, S or NR^{a};
R^{a} is alkyl;
R¹ is selected from the group consisting of H and aryl;
R² is selected from the group consisting of carboxyl, aminocarbonyl, alkylsulfonylaminocarbonyl and alkoxycarbonyl;
R³ is selected from the group consisting of haloalkyl, alkyl, aralkyl, cycloalkyl and aryl optionally substituted with one or more radicals selected from alkylthio, nitro and alkylsulfonyl; and
each R⁴ is independently selected from the group consisting of H, halo, alkyl, aralkyl, alkoxy, aryloxy, heteroaryloxy, aralkyloxy, heteroaralkyloxy, haloalkyl, haloalkoxy, alkylamino, arylamino, aralkylamino, heteroarylamino, heteroarylalkylamino, nitro, amino, aminosulfonyl, alkylaminosulfonyl, arylaminosulfonyl, heteroarylaminosulfonyl, aralkylaminosulfonyl, heteroaralkylaminosulfonyl, heterocyclosulfonyl, alkylsulfonyl, hydroxyarylcarbonyl, nitroaryl, optionally substituted aryl, optionally substituted heteroaryl, aralkylcarbonyl, heteroarylcarbonyl, arylcarbonyl, aminocarbonyl, and alkylcarbonyl; and
R⁴ together with the carbon atoms to which it is attached and the remainder of ring E forms a naphthyl radical; and
(b) an anti-herpes virus agent selected from the group consisting of ganciclovir, foscarnet, cidofovir, acycloguanosine, tri-fluorothymidine, acyclovir, famciclovir, and valaciclovir or an isomer, a pharmaceutically acceptable salt, ester, or prodrug thereof.

53. The composition of claim 52 wherein the cyclooxgyenase-2 selective inhibitor is (S)-6,8-dichloro-2-(trifluoromethyl)-2H-1-benzopyran-3-carboxylic acid.

54. The composition of claim 52 wherein the anti-herpes virus agent is ganciclovir.

55. The composition of claim 52 wherein the anti-herpes virus agent is foscarnet.

56. The composition of claim 52 wherein the anti-herpes virus agent is cidofovir.

57. The composition of claim 52 wherein the anti-herpes virus agent is acyclovir.

58. The composition of claim 52 wherein the anti-herpes virus agent is famciclovir.

59. The composition of claim 52 wherein the anti-herpes virus agent is valaciclovir.

60. A composition comprising:
(a) a cyclooxygenase-2 selective inhibitor or an isomer, a pharmaceutically acceptable salt, ester, or prodrug thereof, the inhibitor having the formula: 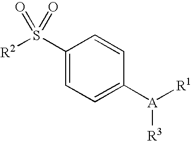
wherein:
A is selected from the group consisting of partially unsaturated or unsaturated heterocyclyl and partially unsaturated or unsaturated carbocyclic rings;
R¹ is selected from the group consisting of heterocyclyl, cycloalkyl, cycloalkenyl and aryl, wherein R¹ is optionally substituted at a substitutable position with one or more radicals selected from alkyl, haloalkyl, cyano, carboxyl, alkoxycarbonyl, hydroxyl, hydroxyalkyl, haloalkoxy, amino, alkylamino, arylamino, nitro, alkoxyalkyl, alkylsulfinyl, halo, alkoxy and alkylthio;
R² is selected from the group consisting of methyl or amino; and
R³ is selected from the group consisting of a radical selected from H, halo, alkyl, alkenyl, alkynyl, oxo, cyano, carboxyl, cyanoalkyl, heterocyclyloxy, alkyloxy, alkylthio, alkylcarbonyl, cycloalkyl, aryl, haloalkyl, heterocyclyl, cycloalkenyl, aralkyl, heterocyclylalkyl, acyl, alkylthioalkyl, hydroxyalkyl, alkoxycarbonyl, arylcarbonyl, aralkylcarbonyl, aralkenyl, alkoxyalkyl, arylthioalkyl, aryloxyalkyl, aralkylthioalkyl, aralkoxyalkyl, alkoxyaralkoxyalkyl, alkoxycarbonylalkyl, aminocarbonyl, aminocarbonylalkyl, alkylaminocarbonyl, N-arylaminocarbonyl, N-alkyl-N-arylaminocarbonyl, alkylaminocarbonylalkyl, carboxyalkyl, alkylamino, N-arylamino, N-aralkylamino, N-alkyl-N-aralkylamino, N-alkyl-N-arylamino, aminoalkyl, alkylaminoalkyl, N-arylaminoalkyl, N-aralkylaminoalkyl, N-alkyl-N-aralkylaminoalkyl, N-alkyl-N-arylaminoalkyl, aryloxy, aralkoxy, arylthio, aralkylthio, alkylsulfinyl, alkylsulfonyl, aminosulfonyl, alkylaminosulfonyl, N-arylaminosulfonyl, arylsulfonyl, N-alkyl-N-arylaminosulfonyl; and
(b) an anti-herpes virus agent selected from the group consisting of ganciclovir, foscamet, cidofovir, acycloguanosine, tri-fluorothymidine, acyclovir, famciclovir, and valaciclovir or an isomer, a pharmaceutically acceptable salt, ester, or prodrug thereof.

61. The composition of claim 60 wherein the anti-herpes virus agent is ganciclovir.

62. The composition of claim 60 wherein the anti-herpes virus agent is foscamet.

63. The composition of claim 60 wherein the anti-herpes virus agent is cidofovir.

64. The composition of claim 60 wherein the anti-herpes virus agent is acyclovir.

65. The composition of claim 60 wherein the anti-herpes virus agent is famciclovir.

66. The composition of claim 60 wherein the anti-herpes virus agent is valaciclovir.

67. A composition comprising:
(a) a cyclooxygenase-2 selective inhibitor or an isomer, a pharmaceutically acceptable salt, ester, or prodrug thereof, the inhibitor having the formula: 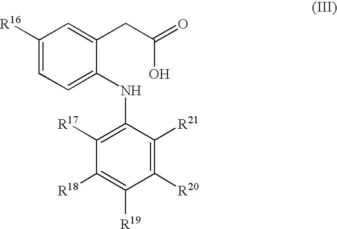
wherein:
R¹⁶ is methyl or ethyl;
R¹⁷ is chloro or fluoro;
R¹⁸ is hydrogen or fluoro;
R¹⁹ is hydrogen, fluoro, chloro, methyl, ethyl, methoxy, ethoxy or hydroxy;
R²⁰ is hydrogen or fluoro;
R²¹ is chloro, fluoro, trifluoromethyl or methyl; and
(b) an anti-herpes virus agent selected from the group consisting of ganciclovir, foscarnet, cidofovir, acycloguanosine, tri-fluorothymidine, acyclovir, famciclovir, and valaciclovir or an isomer, a pharmaceutically acceptable salt, ester, or prodrug thereof.

68. The composition of claim 67 wherein the anti-herpes virus agent is ganciclovir.

69. The composition of claim 67 wherein the anti-herpes virus agent is foscarnet.

70. The composition of claim 67 wherein the anti-herpes virus agent is cidofovir.

71. The composition of claim 67 wherein the anti-herpes virus agent is acyclovir.

72. The composition of claim 67 wherein the anti-herpes virus agent is famciclovir.

73. The composition of claim 67 wherein the anti-herpes virus agent is valaciclovir.

74. A composition comprising a cyclooxygenase-2 selective inhibitor selected from the group consisting of celecoxib, deracoxib, valdecoxib, rofecoxib, lumiracoxib, etoricoxib, parecoxib, 2-(3,4-difluorophenyl)-4-(3-hydroxy-3-methylbutoxy)-5-[4-(methylsulfonyl)phenyl]-3(2H)-pyridazinone, and (S)-6,8-dichloro-2-(trifluoromethyl)-2H-1-benzopyran-3-carboxylic acid; and
an anti-herpes virus agent selected from the group consisting of ganciclovir, foscamet, cidofovir, acycloguanosine, tri-fluorothymidine, acyclovir, famciclovir, and valaciclovir.

75. The composition of claim 74 wherein the cyclooxyenase-2 selective inhibitor is selected from the group consisting of celecoxib, deracoxib, valdecoxib, rofecoxib, lumiracoxib, etoricoxib, and parecoxib; and the anti-herpes virus agent is selected from the group consisting of acyclovir, famciclovir, and valaciclovir.
